(19) United States
(12) Patent Application Publication
(10) Pub. No.: US 2010/0099871 A1
(43) Pub. Date: Apr. 22, 2010

(54) ANTI-PSYCHOTIC COMPOUNDS AND PHARMACEUTICAL COMPOSITIONS THEREOF

(76) Inventors: Dwight W. Miller, White Hall, AR (US); Neil C. Mitchell, Maumelle, AR (US); Bill W. Massey, Ramona, CA (US)

Correspondence Address: WINSTEAD PC, P.O.Box 50784, Dallas, TX, 75201, US

(21) Appl. No.: 12/542,556

(22) Filed: Aug. 17, 2009

Related U.S. Application Data
(60) Provisional application No. 61/089,408, filed on Aug. 15, 2008.
(60) Provisional application No. 61/092,600, filed on Aug. 28, 2008.
(60) Provisional application No. 61/100,456, filed on Sep. 26, 2008.
(60) Provisional application No. 61/104,929, filed on Oct. 13, 2008.

(51) Int. Cl. C07D 401/06; C07D 207/09

(52) U.S. Cl. 544/361; 548/566

(57) ABSTRACT

Compounds having anti-psychotic activity are disclosed herein. In some embodiments, the compounds are atypical anti-psychotics. In some embodiments, the compounds are selective for the 5-HT_{2A} receptor over the D₂ receptor. Pharmaceutical derivatives and pharmaceutical compositions including the compounds are disclosed herein. Methods for treating a psychotic condition that include administering the compounds, a pharmaceutical derivative thereof, or a pharmaceutical composition thereof are also disclosed herein.

### CROSS-REFERENCE TO RELATED APPLICATIONS

This application claims priority to U.S. provisional patent applications 61/089,408 filed Aug. 15, 2008; 61/092,600 filed Aug. 28, 2008; 61/100,456 filed Sep. 26, 2008 and 61/104,929 filed Oct. 13, 2008. Each of these provisional patent patent applications is incorporated by reference herein in its entirety.

### STATEMENT REGARDING FEDERALLY SPONSORED RESEARCH

Not applicable.

### BACKGROUND

Antipsychotics are a group of drugs often used to treat psychosis. An illustrative psychotic condition is schizophrenia. A number of different types of antipsychotics have been developed, particularly for treating schizophrenia. A first generation of antipsychotic drugs is commonly known in the art as typical antipsychotics based on their mechanism of action. A second generation of antipsychotic drugs is commonly referred to in the art as atypical antipsychotics. Illustrative FDA-approved atypical antipsychotics include, but are not limited to, Clozapine, Risperidone, Olanzapine, Quetiapine, Ziprasidone, Aripiprazole, and Paliperidone. FDA approved indications for antipsychotics include, but are not limited, to acute mania, bipolar mania, psychotic agitation, and bipolar maintenance.

Both classes of antipsychotics function through inhibition of dopamine receptors in the brain. Antipsychotic drugs also encompass a wide range of receptor specificity. Most, but not all atypical antipsychotics, also interact with serotonin receptors (e.g., 5-HT_{1A}, 5-HT_{2A} and 5-HT_{3A}) in addition to dopamine receptors (e.g., D₂ and D₄). Some atypical antipsychotics are known to be partial agonists for serotonin receptors, such as 5-HT_{1A} and 5-HT_{2A}. Characteristics of atypical antipsychotics compared to typical antipsychotics may include a decreased propensity to cause Extrapyramidal Side Effects and lack of sustained prolactin elevation.

Although atypical antipsychotics are generally thought to be preferable to typical antipsychotics, a number of side effects have been observed for both antipsychotic classes, including, for example, weight gain, insulin resistance, hypergylcemia, increased lipid levels, agranulocytosis, and tardive dyskinesia. Agranulocytosis is especially problematic for Clozapine, an atypical antipsychotic, which is one of the more effective drugs for treating schizophrenia, particularly schizophrenia which is treatment resistant to other drugs. As a result, patients being treated with Clozapine are required to undergo weekly blood testing to monitor for the presence of agranulocytosis.

In view of the foregoing, indentification of more effective antipsychotic drugs for treating schizophrenia and related psychotic conditions would be of substantial value. In particular, discovery of antipsychotic drugs having a reduced incidence of side effects, especially agranulocytosis, would be of particular benefit.

### SUMMARY

In various embodiments, the present disclosure provides compounds that have structures selected from:

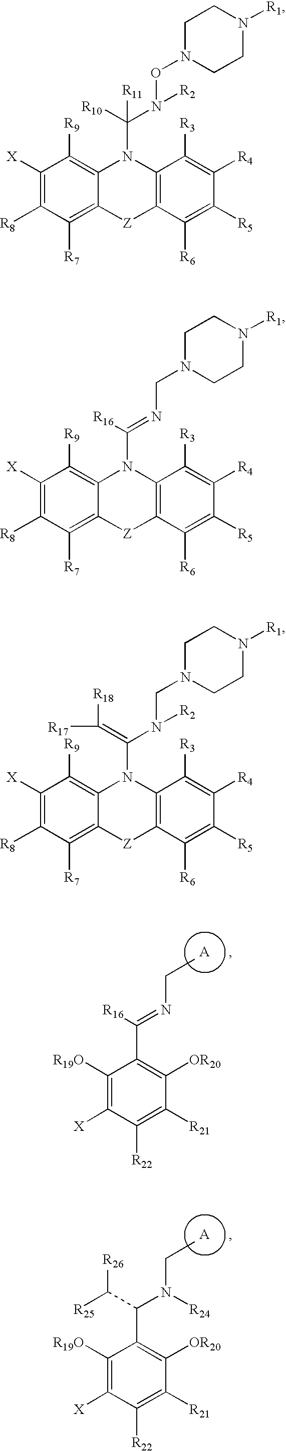

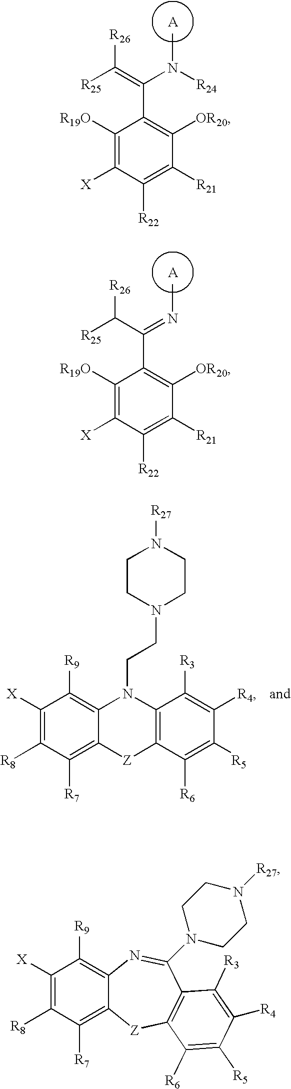

including tautomers, stereoisomers, geometric isomers, and pharmaceutical derivatives thereof. Variables X, Z, and R₁-R₂₇ are detailed herein. Choice of the variables is conducted such that the compounds display antipsychotic activity in various embodiments of the disclosure. The antipsychotic activity may be typical or atypical.

Methods for treating a psychotic condition by administering any of the compounds, any pharmaceutical derivative of the compounds, or any pharmaceutical composition of the compounds are also disclosed herein. Pharmaceutical compositions of the compounds and pharmaceutical compositions of pharmaceutical derivatives of the compounds are also disclosed herein.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

For a more complete understanding of the present disclosure, and the advantages thereof, reference is now made to the following descriptions to be taken in conjunction with the accompanying drawings describing a specific embodiment of the disclosure, wherein:

FIG. 1 presents a plot of predicted versus experimental K_{d} for the D₂ receptor;

FIG. 2 presents a plot of predicted versus experimental K_{d} for the 5-HT_{2A} receptor;

FIG. 3 presents a plot of predicted versus experimental K_{d} for the 5-HT_{1A} receptor;

FIG. 4 presents a plot of predicted versus experimental IC₅₀ for hERG inhibition;

FIG. 5 presents a plot of predicted versus experimental antipsychotic-associated weight gain;

FIG. 6 presents a plot of predicted versus experimental agranulocytosis relative risk;

FIG. 7 presents a Certificate of Analysis for LMD-00060;

FIG. 8 presents a Certificate of Analysis for LMD-00076;

FIG. 9 shows the ¹H NMR of LMD-00100t;

FIG. 10 shows the LC-MS of LMD-00100t;

FIG. 11 shows a plot of pre-frontal cortex dopamine release as a function of time following administration of LMD-00076;

FIG. 12 shows a plot of nucleus accumbens dopamine release as a function of time following administration of LMD-00076;

FIG. 13 shows a plot of pre-frontal cortex acetylcholine release as a function of time following administration of LMD-00076;

FIG. 14 shows a plot of pre-frontal cortex dopamine release as a function of time following administration of LMD-00100t; and

FIG. 15 shows a plot of nucleus accumbens dopamine release as a function of time following administration of LMD-00100t.

### DETAILED DESCRIPTION

In the following description, certain details are set forth such as specific quantities, sizes, etc. so as to provide a thorough understanding of the present embodiments disclosed herein. However, it will be obvious to one of ordinary skill in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing particular embodiments of the disclosure and are not intended to be limiting thereto.

### DEFINITIONS

While most of the terms used herein will be recognizable to those of skill in the art, the following definitions are nevertheless put forth to aid in the understanding of the present disclosure. It should be understood, however, that when not explicitly defined, terms should be interpreted as adopting a meaning presently accepted by those of skill in the art.

“Alkali metal,” as defined herein, comprises a metal from group 1 of the periodic table, such as but not limited to lithium, sodium, and potassium.

“Alkaline earth metal,” as defined herein, comprises a metal from group 2 of the periodic table, such as but not limited to magnesium, calcium, and barium.

“Atypical antipsychotics,” as defined herein, comprise a group of structurally unrelated drugs that are distinguished from typical antipsychotics, also known as first generation antipsychotics, in their mechanism of action. First generation antipsychotics typically comprise neuroleptics and tranquilizers. Most atypical antipsychotics share a common attribute of interacting with serotonin receptors and dopamine receptors. However, interaction with both types of receptors is not necessary to make a drug an atypical antipsychotic. Receptor agonism, partial agonism, and antagonism are all demonstrated by atypical antipsychotics. Characteristics of atypical antipsychotics may include a decreased propensity to cause Extrapyramidal Side Effects and lack of sustained prolactin elevation.

“Pharmaceutical derivatives,” as defined herein, include salts, esters, enol ethers, enol esters, acetals, ketals, orthoesters, hemiacetals, hemiketals, acids, bases, solvates, hydrates and prodrugs of a parent compound. Said derivatives may be readily prepared using known methods of derivatization by those skilled in the art. The compounds may be administered to a subject without substantial toxic effects. The pharmaceutical derivatives are either themselves pharmacologically active or are activated following in vivo cleavage. In other words, the pharmaceutical derivatives may comprise a prodrug form of the parent drug. Pharmaceutically acceptable salts may include, but are not limited to, amine salts, including but not limited to, N,N′-dibenzylethylenediamine, chloroprocaine, choline, ammonia, diethanolamine and related hydroxyalkylamines, ethylenediamine, N-methylglucamine, procaine, N-benzylphenethylamine, 1-p-chlorobenzyl2-pyrrolidin-1′-ylmethylbenzimidazole, diethylamine and related alkylamines, piperizine, tris(hydroxymethyl)aminomethane, alkali metal salts, alkaline earth metal salts, transition metal salts, such as but not limited to zinc; inorganic salts such as but not limited to sodium hydrogen phosphate, disodium phosphate, potassium hydrogen phosphate, dipotassium hydrogen phosphate, fluoride, chloride, bromide, iodide, sulfate; and organic acid salts, such as but not limited to acetates, lactates, malates, tartrates, citrates, ascorbates, succinates, butyrates, valerates, mesylates, and fumarates. Pharmaceutically acceptable esters may include, but are not limited to alkyl, alkenyl, alkynyl, aryl, aralkyl, and cycloalkyl esters of acidic groups, including but not limited to, carboxylic acids, phosphoric acids, phosphinic acids, sulfonic acids, sulfinic acids, and boronic acids. Pharmaceutically acceptable enol ethers may include, but are not limited to, derivatives of the formula C═C(OR), wherein R is hydrogen, alkyl, alkenyl, alkynyl, aryl, aralkyl, and cycloalkyl. Pharmaceutically acceptable enol esters may include, but are not limited to, derivatives of the formula C═C(OC(═O)R), wherein R is hydrogen, alkyl, alkenyl, alkynyl, aryl, aralkyl, and cycloalkyl. Pharmaceutically acceptable solvates and hydrates comprise complexes of a compound molecule and at least one solvent or water molecule. In some embodiments, the solvate or hydrate comprises about 1 compound molecule and about 100 solvent or water molecules. In other embodiments, the solvate or hydrate comprises about 1 compound molecule and about 10 solvent or water molecules. In still other embodiments, the solvate or hydrate comprises about 1 compound molecule and about 1 to about 5 solvent or water molecules.

“Psychotic conditions,” as defined herein, include but are not limited to, schizophrenia, schizoaffective disorder, schizophreniform disorder, brief psychotic disorder, delusional, shared psychotic disorder (Folie à deux), substance induced psychosis, psychosis due to a general medical condition, and psychosis due to a not otherwise specified condition. Symptoms of psychotic conditions include, but are not limited to, hallucinations, delusions, thought disorder and lack of insight. Pyschotic conditions may arise without limitation from psychiatric or general medical conditions.

“Treating,” as defined herein, comprises administering a compound to ameliorate or otherwise beneficially alter a disease state. In some embodiments, treating comprises administering a compound disclosed herein to beneficially alter a psychotic condition. Treating also comprises administering a pharmaceutical derivative or pharmaceutical composition disclosed herein to beneficially alter a psychotic condition. Ameliorating or beneficially altering may be permanent or temporary.

The chemical nomenclature terms and descriptions that follow will be recognizable to those of ordinary skill in the art. It should be understood that when not explicitly defined, terms should be interpreted as adopting a meaning presently accepted by those of skill in the art. To aid in understanding of the present disclosure, the following chemical nomenclature terms are put forth.

“Alkoxy,” as defined herein, refers to OR, wherein R is an alkyl group.

“Alkoxycarbonyl,” as defined herein, refers to C(═O)OR, wherein R is an alkyl group.

“Alkylthio,” as defined herein, refers to SR, wherein R is an alkyl group.

“Aminocarbonyl,” as defined herein, refers to C(═O)NH₂. Aminocarbonyl groups may be substituted on nitrogen with one or two substituents, such as but not limited to alkyl, alkenyl, alkynyl, aryl, aralkyl, heteroalkyl, cycloalkyl, heterocyclyl, heterocyclylalkyl, heteroaryl, or heteroaralkyl groups. “Alkylaminocarbonyl, alkylarylaminocarbonyl, arylaminocarbonyl, dialkylaminocarbonyl, and diarylaminocarbonyl” refer to embodiments of such indicated substitution.

“Aralkyl,” as defined herein, refers to an alkyl group in which one or more of the hydrogen atoms of the parent alkyl group have been replaced with an aryl group.

“Alkyl, alkenyl, and alkynyl,” as defined herein, refer to groups that contain from 1 to about 20 carbon atoms, if not otherwise specified. Said groups may be straight or branched. Alkyl groups contain from 1 to about 20 or 1 to about 16 carbon atoms and may be straight or branched. Alkenyl groups contain from 2 to about 20 or 2 to about 16 carbon atoms, contain 1 to 10 or 1 to 8 double bonds, and may be straight or branched. Alkynyl groups contain from 2 to about 20 or 2 to about 16 carbon atoms, contain 1 to 10 or 1 to 8 triple bonds, and may be straight or branched. Illustrative alkyl, alkenyl, and alkynyl groups may include, but are not limited to, methyl, ethyl, propyl, isopropyl, butyl, isobutyl, s-butyl, t-butyl, vinyl, propenyl, ethynyl, and propynyl.

“Aryl,” as defined herein, refers to monocyclic or polycyclic aromatic groups containing 6 to about 18 carbon atoms. Illustrative but non-limiting examples of aryl groups include, phenyl, naphthyl, and fluorenyl.

“Aryloxy,” as defined herein, refers to OR, wherein R is an aryl group.

“Arylthio,” as defined herein, refers to SR, wherein R is an aryl group.

“Aryloxycarbonyl,” as defined herein, refers to C(═O)—OR, wherein R is an aryl group.

“Cycloalkyl,” as defined herein, refers to a saturated or unsaturated monocyclic or multicyclic ring systems having 3 to about 10 carbon atoms. In some embodiments, a cycloalkyl group may have 3 to 6 carbon atoms. Unsaturated cycloalkyl groups may contain at least one double bond or one triple bond. Unsaturated cycloalkyl groups may alternatively contain at least one double bond and at least one triple bond. Multicyclic ring systems may be fused, bridged, or connected in a spiro fashion.

“Cycloalkylalkyl,” as defined herein, refers to an alkyl group in which one of the hydrogen atoms of the parent alkyl group has been replaced with a cycloalkyl group.

“Halogen, halo, and halide,” as defined herein, refer to F, Cl, Br, and I. “Pseudohalide,” as used herein, refers to groups that behave chemically and pharmacologically with substantial similarity to halides. Pseudohalides may include, but are not limited to, cyano, thiocyanato, azido, trifluoromethyl, trifluoromethoxy, perfluoroalkyl, and perfluoroalkoxy groups.

“Heteroalkyl,” as defined herein, refers to straight, branched, or cyclic alkyl, alkenyl, alkynyl, or cycloalkyl groups having at least one heteroatom inserted in the hydrocarbon chain. Heteroatoms may include, but are not limited to, oxygen, sulfur (including S═O and SO₂ groups), and nitrogen (including NH, NR, and N⁺RR′ groups), wherein the R substituents may independently be alkyl, aryl, alkenyl, alkynyl, aralkyl, heteroalkyl, cycloalkyl, cycloalkylalkyl, heterocyclyl, heterocyclylalkyl, heteroaryl, heteroarylalkyl, SO₂R′, or C(═O)R′, wherein R′ may be alkyl, aryl, alkenyl, alkynyl, aralkyl, heteroalkyl, cycloalkyl, cycloalkylalkyl, heterocyclyl, heterocyclylalkyl, heteroaryl, heteroarylalkyl, OY, or NYY′, wherein Y and Y′ are independently H, alkyl, aryl, alkenyl, alkynyl, aralkyl, heteroalkyl, cycloalkyl, cycloalkylalkyl, heterocyclyl, heterocyclylalkyl, heteroaryl, and heteroarylalkyl. In an embodiment, heteroalkyl groups may have from 1 to about 20 atoms in the chain.

“Heteroarylalkyl group,” as defined herein, refers to an alkyl group in which one or more of the hydrogen atoms of the alkyl group has been replaced by a heteroaryl group.

“Heteroaryl group,” as defined herein, refers to monocyclic or multicyclic aromatic groups having 5 to about 14 atoms, wherein at least one of the atoms comprising the monocyclic or multicyclic aromatic ring is replaced by a heteroatom. In some embodiments, 1 to about 4 heteroatoms comprise the heteroaryl group.

“Heteroatom,” as defined herein, may include but is not limited to oxygen, nitrogen, and sulfur.

“Heterocyclyl group,” as defined herein, refers to monocyclic or multicyclic non-aromatic groups having 3 to about 14 atoms, wherein at least one of the atoms comprising the monocyclic or multicyclic aromatic ring is replaced by a heteroatom. In some embodiments, 1 to about 4 heteroatoms comprise the heterocyclyl group.

“Heterocyclylalkyl group,” as defined herein, refers to an alkyl group in which one or more of the hydrogen atoms of the alkyl group has been replaced by a heterocyclyl group.

“Oxo,” as defined herein, refers to ═O.

“Thioxo,” as defined herein, refers to ═S.

It is to be understood that in any of the embodiments disclosed herein that alkyl, alkenyl, alkynyl, heteroalkyl, cycloalkyl, cycloalkylalkyl, aryl, aralkyl, heteroaryl, heteroarylalkyl, heterocyclyl, and heterocyclylalkyl groups may be further substituted. In some embodiments, said groups are substituted with one substituent selected from Q₁. In other embodiments, said groups are substituted with 2-4 substituents independently selected from Q₁. Substitution may occur on carbon or on any heteroatom comprising the group that may be functionalized. As will be evident to one having skill in the art the choice of Q₁ may determine the position at which Q₁ substitution may occur. In embodiments disclosed herein, Q₁ may be selected from the group consisting of halo, pseudohalo, hydroxy, oxo, thioxo, cyano, nitro, formyl, mercapto, hydroxycarbonyl, hydroxycarbonylalkyl, alkyl, aminoalkyl, diaminoalkyl, alkenyl, alkynyl, cycloalkyl, cycloalkylalkyl, heterocyclyl, heterocyclylalkyl, aryl, heteroaryl, aralkyl, aralkenyl, aralkynyl, heteroarylalkyl, alkylidene, arylalkylidene, alkylcarbonyl, arylcarbonyl, heterarylcarbonyl, alkoxycarbonyl, aryloxycarbonyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkyoxycarbonyoxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N-dialkyureido, N-alkyl-N′-arylureido, N-aryl-N′-alkylureido, N,N′-diarylureido, N,N′,N′-trialkylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonyl amino, aryloxycarbonylamino, alkylsulfonylamino, arylsulfonylamino, heteroarylsulfonylamino, heterocyclylsulfonylamino, heteroarylthio, azido, alkyl and aryl ammonium salts, alkylthio, arylthio, perfluoroalkylthio, hydroxycarbonylalkylthio, thiocyanato, isothiocyanato, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl, alkylarylaminosulfonyl. Two Q₁ groups may substitute alkyl, alkenyl, alkynyl, heteroalkyl, cycloalkyl, cycloalkylalkyl, aryl, aralkyl, heteroaryl, heteroarylalkyl, heterocyclyl, and heterocyclylalkyl groups disclosed herein in either a 1,2- or 1,3-arrangement to form a ring, wherein the ring comprises an alkylene group ((—CH₂)_{y}—), alkyleneoxy, (—O—(CH₂)_{y}—), alkylenethioxy (—S—(CH₂)_{y}—), alkylenedioxy (—O—(CH₂)_{y}—O—), thioalkyleneoxy (—S—(CH₂)_{y}—O—), or alkylenedithioxy (—S—(CH₂)_{y}—S—), and wherein y is 1 or 2. Two Q1 groups may also substitute the same atom to form an alkylene.

It is to be understood that any of the compounds herein may contain chiral centers. Such chiral centers may be of either the R or S configuration or a mixture thereof. The compounds may be enantiomerically pure, diastereomerically pure, enantiomeric mixtures, or diasteromeric mixtures. For compounds that are epimerizable, administration of a compound in its R form is equivalent to administration of the compound in its S form. It is to be understood that references made to the compounds herein are to single compounds. However, it is to be understood that a single compound may comprise a mixture of stereoisomers. Certain compounds provided herein are substantially pure, meaning that the compounds are sufficiently homogenous to appear free of readily detectable impurities through analytical methods commonly used by those of skill in the art. Likewise, a substantially pure compound may comprise a mixture of stereoisomers. Unless otherwise specified, double bonds may exist in both E and Z geometric isomers, both geometric isomers residing within the spirit and scope the instant disclosure. Unless otherwise specified, tautomeric forms of any compound disclosed herein reside within the spirit and scope of the instant disclosure

### Spectral Modeling to Identify Antipsychotic Compounds and Validation Studies:

In various embodiments, compounds having antipsychotic activity are disclosed herein.

The compounds were identified through an in silico screening method based on spectral data (hereinafter referred to as spectral modeling) to find compounds potentially having a D₂/5-HT_{2A} receptor mechanism of action. In some embodiments, the compounds have a greater affinity for 5-HT_{2A} than for D₂. In some embodiments, the compounds display atypical antipsychotic activity.

Spectral modeling is founded on the fundamental principles of molecular spectroscopy. Spectral data, which are characteristic of the electronic and geometrical configurations of the molecule, are will described by the basic principles of quantum mechanics. Spectral modeling may utilize the spectral attributes of known molecules and relate these spectral attributes to biological activity. Once a spectral model with apparent predictive value has been developed, new molecules may be tested by the spectral model that have no apparent structural similarity to the training set and validation molecules.

The spectral modeling method herein makes use of ¹³C NMR data and provides several advantages over crystallographic data typically used in QSAR predictive methods, which are commonly used in drug discovery. These advantages include: 1) ¹³C NMR spectral measurements are in solution, reflecting a biological environment; 2) spectral data is quantitative and not subject to human judgment; and 3) spectral data is very sensitive to small changes in molecular structure. Furthermore, ¹³C NMR spectra are readily predictable based on known molecular parameters. Spectral modeling describes the activity of interest as a function of the ¹³C spectral attributes of the compound. The spectral modeling method has very high predictive power (>90%), which allows virtual screening of numerous compounds without the requirement to synthesize and purify the entire library of compounds. In this sense, the spectral modeling method provides time and materials savings for drug discovery efforts. Using the spectral modeling method, lead candidates may be identified in just a few months, greatly expediting the drug discovery process. A spectral model can be built for any property of interest, including but not limited to receptor binding affinity, clinical potency, and toxicity.

Spectral models of certain pharmacological data characteristic of atypical antipsychotic drugs were created to identify the molecules of the present disclosure. For example, models for receptor affinity (D₂, 5-HT_{2A} and 5-HT_{1A}) and toxicity (hERG inhibition, antipsychotic-associated weight gain, and agranulocytosis risk) were constructed. In particular, molecules having higher affinity for 5-HT_{2A} over D₂ were identified. Once suitable training biological data sets for known molecules had been selected, spectral modeling methods were employed to determine the most significant relationships between molecular structure and biological response by generating formalisms that describe the activity of interest as a function of the spectral attributes of the drugs. The models so obtained were subjected to leave-10%-out cross validation and further tested by double-blind data approximately equal in number to 20% of the original data set. The agranulocytosis risk model displayed >95% predictive accuracy at 10% cross validation on 86 molecules. The antipsychotic activity model displayed 100% predictive accuracy at 10% cross validation of 111 molecules. The hERG inhibition model displayed 100% predictive accuracy at 10% cross validation of 88 molecules. Further details concerning the spectral models is presented in the Experimental Examples.

Results from the spectral models were utilized to generate new compounds, which are identified herein. The new compounds were then screened in a battery of completed spectral models to identify potential lead candidates. As disclosed herein, >250 new chemical entities were identified through spectral modeling methods. Based on their predicted antipsychotic activity and predicted receptor binding profiles, three of the compounds were selected for synthesis and further in vitro and in vivo testing.

Three of the compounds predicted to display antipsychotic activity have been synthesized and characterized. Once compounds meeting selection criteria of the spectral models were identified, confirmatory in vitro screening and cross-screening were conducted. For example, receptor binding studies were conducted for 5-HT_{1A}, 5-HT_{2A}, 5-HT_{2C}, 5-HT₆, 5-HT₇, α1A, α1B, α1D, α2A, α2B, α2C, D₂, H₁, M₁, M₂, M₃, M₄, and M₅ to further determine a broad pharmacological profile for compounds meeting spectral modeling selection criteria. Synthesis and characterization of these compounds is provided as an Experimental Example. Experimentally determined receptor binding affinities are shown as an Experimental Example and demonstrate values comparable with those predicted by the in silico models. For D₂ and 5-HT_{2A} receptors, experimental results were within 11% of predicted values. In certain embodiments, the compounds disclosed herein have D₂ and 5-HT_{2A} receptor binding affinities comparable to those displayed by the atypical antipsychotic clozapine. In some embodiments, the compounds are selective for 5-HT_{2A} over D₂. In some embodiments, the compounds disclosed herein do not display agranulocytosis activity. As such the compounds are advantageous over clozapine and may be beneficial for treating psychotic conditions, such as schizophrenia.

### Identity of the Compounds:

In various embodiments, compounds having structure I are described herein:

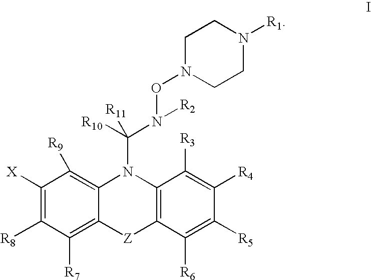

Tautomers, stereoisomers and pharmaceutical derivatives of structure I are also disclosed herein. X is selected from a group consisting of H, F, Cl, and Br. Z is selected from a group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂. R₁₂ is selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₃ is selected from a group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₄ and R₁₅ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio. R₁ and R₂ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl. R₃-R₉ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₁₀ and R₁₁ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, and heteroarylalkyl.

In some embodiments, the compounds have structure II:

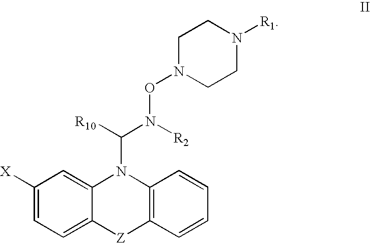

Tautomers, stereoisomers and pharmaceutical derivatives of structure II are disclosed herein.

In some embodiments, the compounds have a structure selected from the group consisting of:

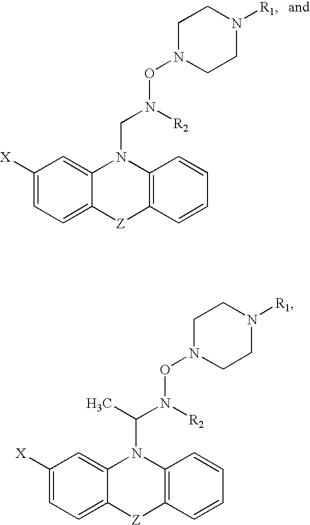

including tautomers and stereoisomers thereof, and pharmaceutical derivatives thereof.

In some embodiments, the compounds have a structure selected from the group consisting of:

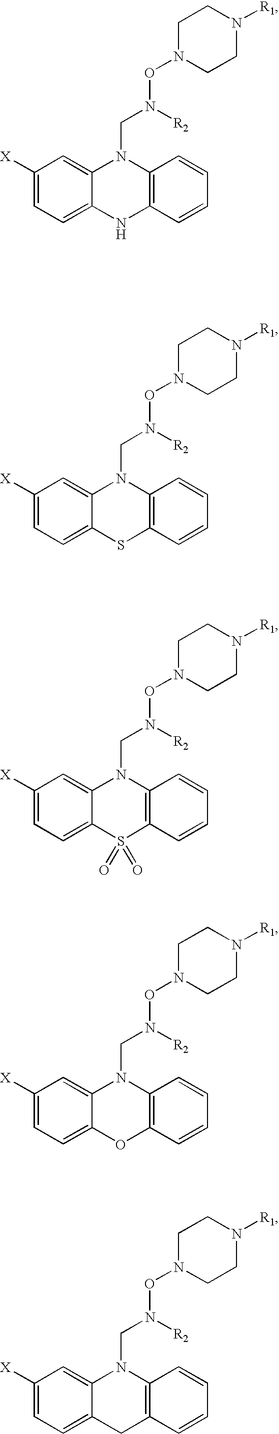

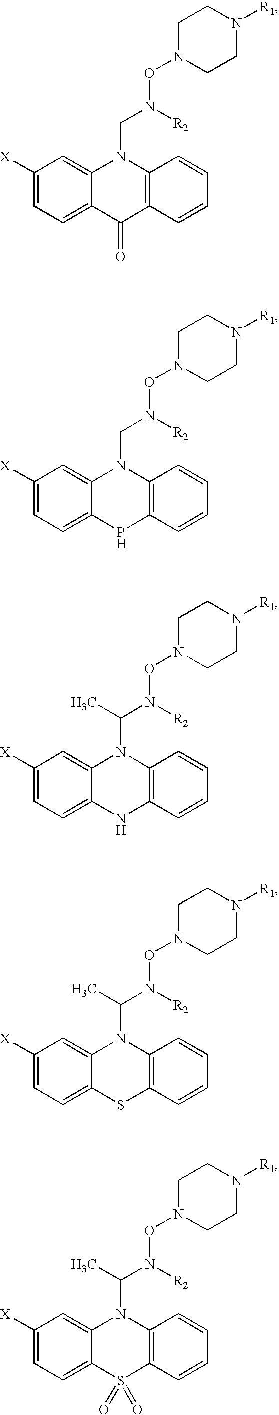

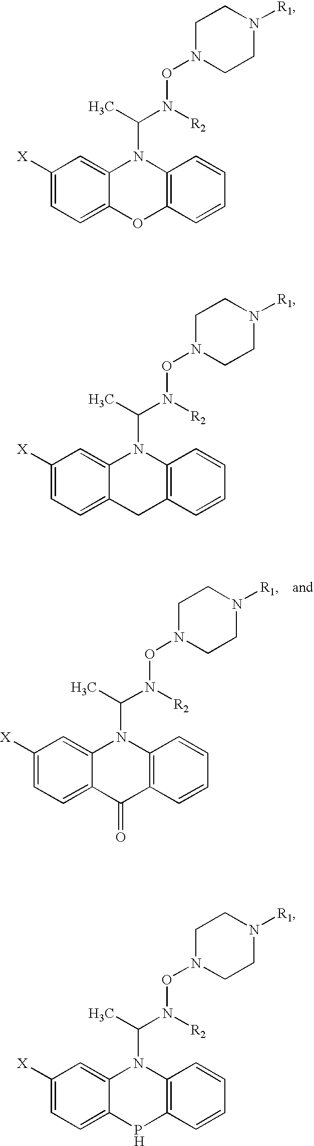

including tautomers and stereoisomers thereof and pharmaceutical derivatives thereof.

In some embodiments, the compounds have a structure selected from the group consisting of:

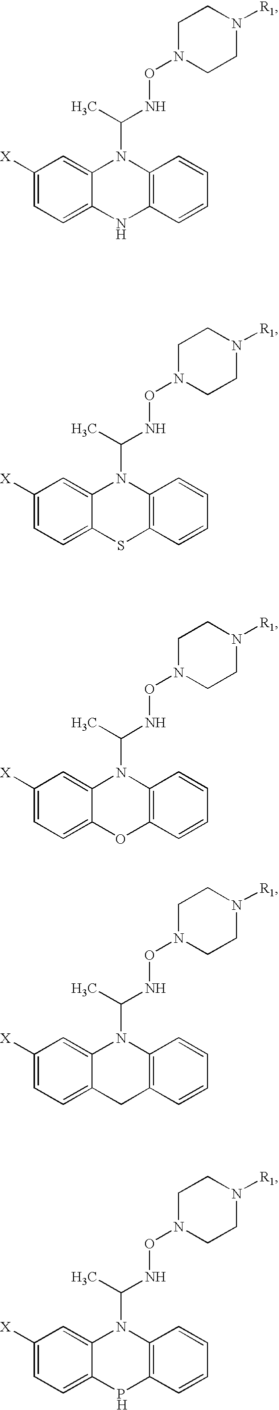

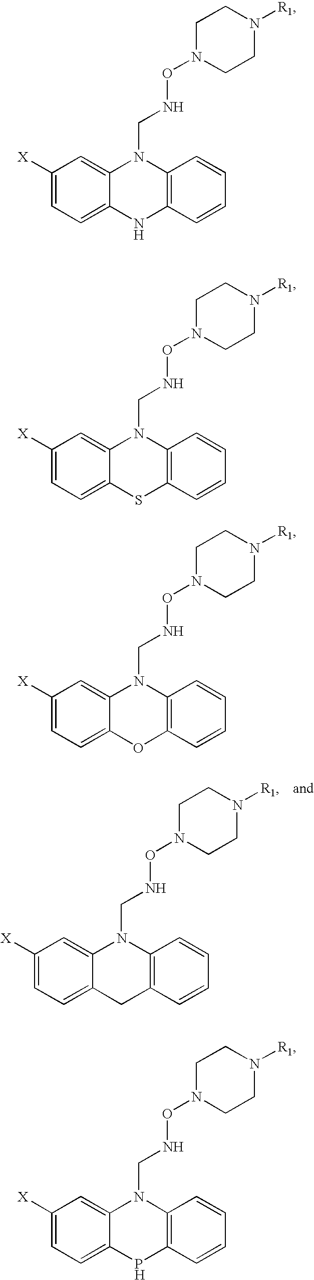

including tautomers and stereoisomers thereof and pharmaceutical derivatives thereof.

In some embodiments, the compounds have a structure selected from the group consisting of:

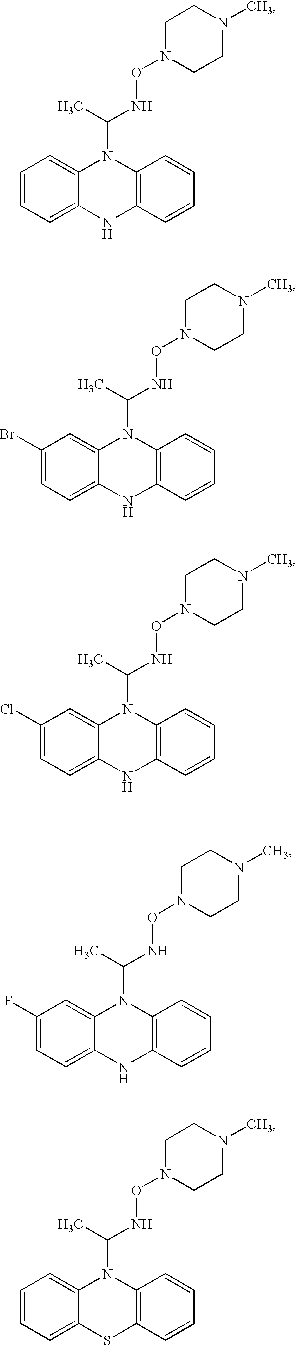

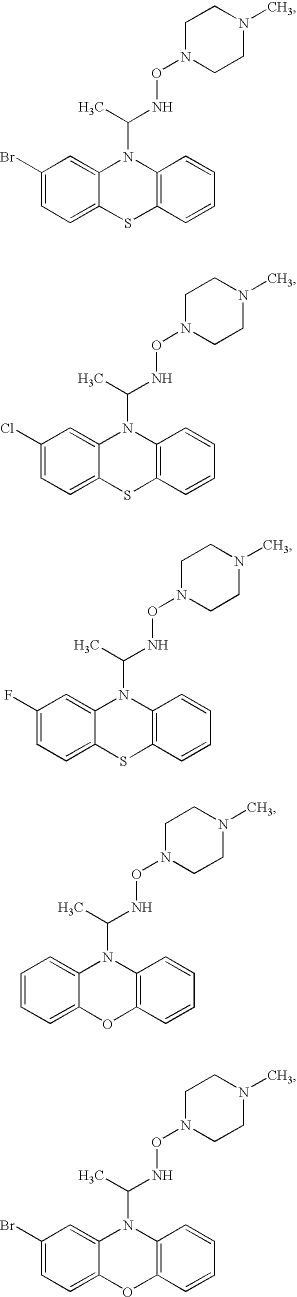

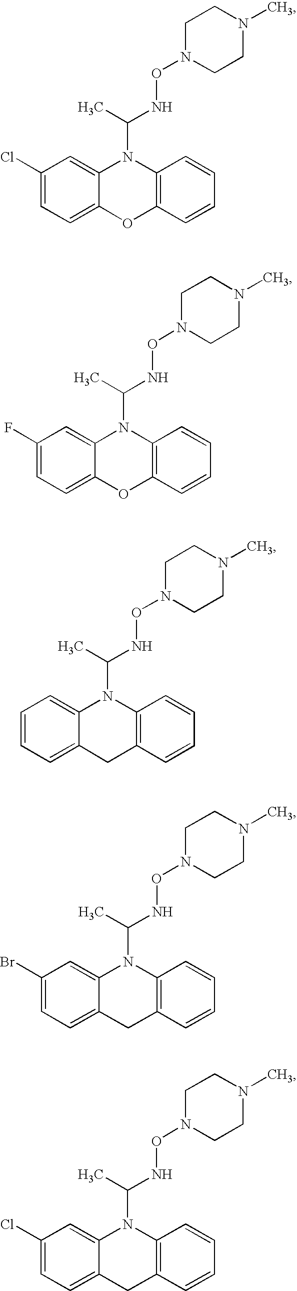

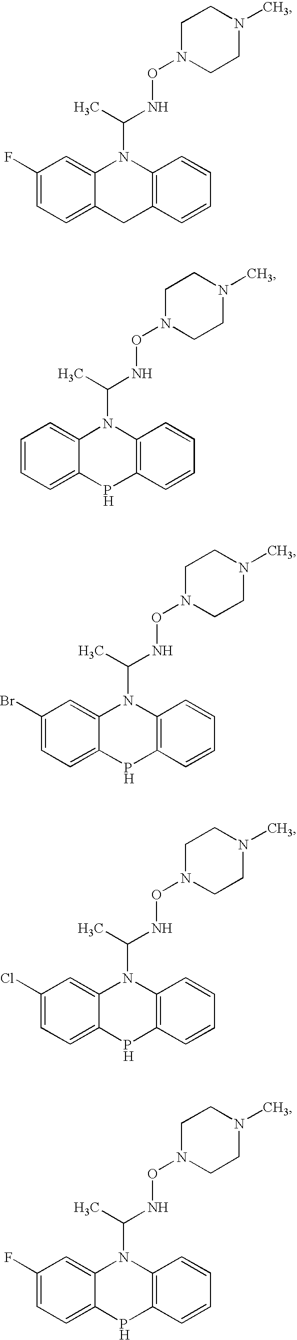

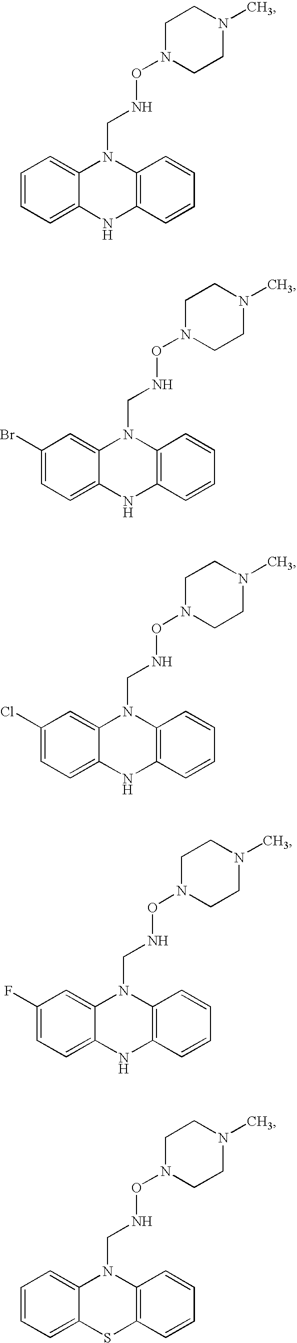

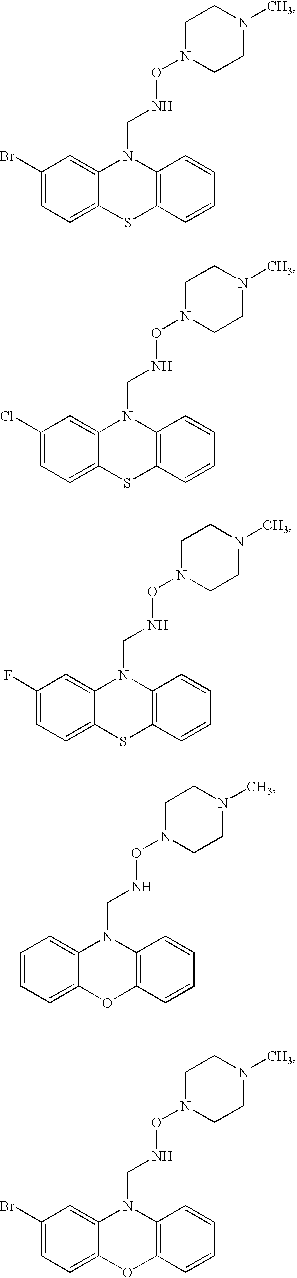

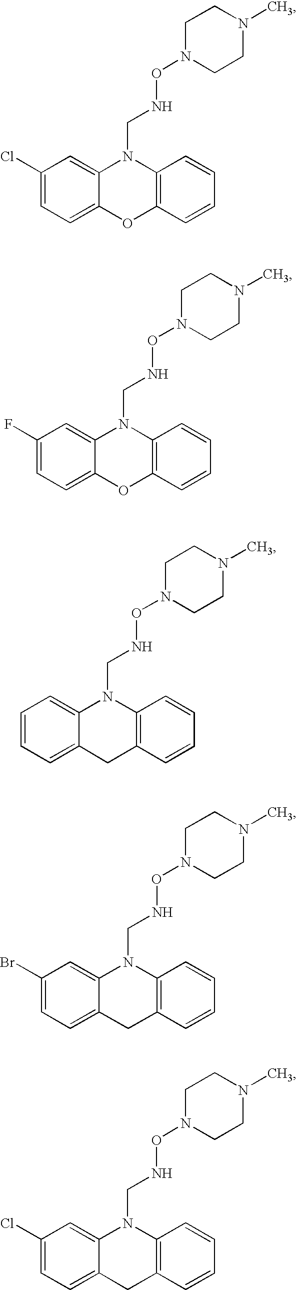

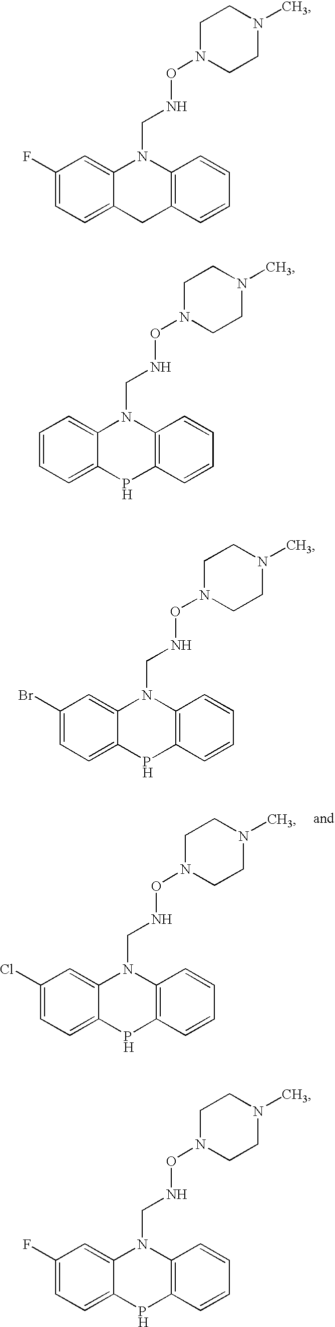

including tautomers and stereoisomers thereof and pharmaceutical derivatives thereof.

In various embodiments, compounds having structure III are described herein:

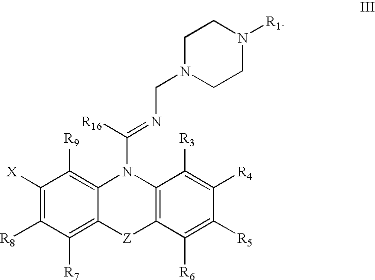

Tautomers, geometric isomers, and pharmaceutical derivatives of structure III are disclosed herein. X is selected from a group consisting of H, F, Cl, and Br. Z is selected from a group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂. R₁₂ is selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₃ is selected from a group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₄ and R₁₅ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio. R₁ is selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl. R₃-R₉ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₁₆ is selected from a group consisting of alkyl, alkenyl, alkynyl, heteroalkyl, aralkyl, cycloalkyl, cycloalkylalkyl, heterocyclyl, heterocyclylalkyl, and heteroarylalkyl.

In some embodiments, the compounds have structure IV:

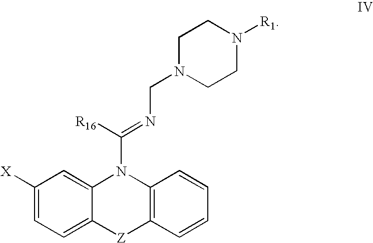

Tautomers, geometric isomers, and pharmaceutical derivatives of structure IV are disclosed herein.

In some embodiments, the compounds have a structure selected from the group consisting of:

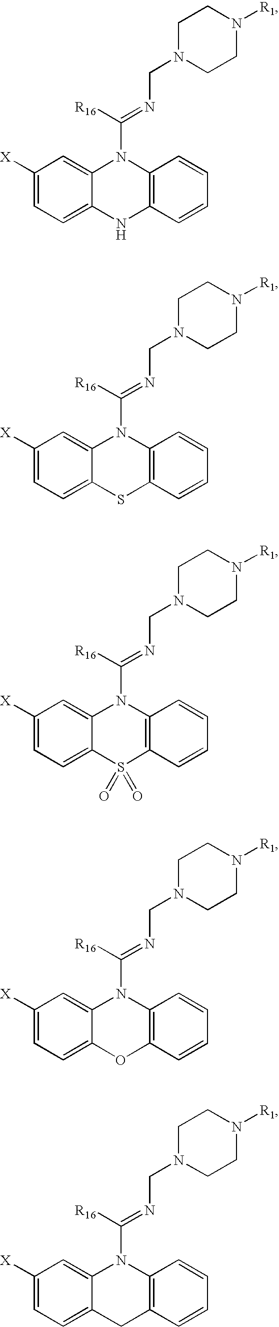

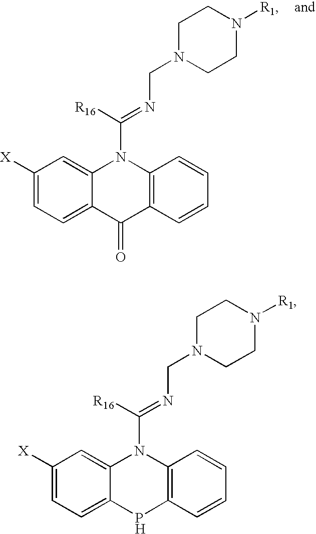

including tautomers and geometric isomers thereof and pharmaceutical derivatives thereof.

In some embodiments, the compounds have a structure selected from the group consisting of:

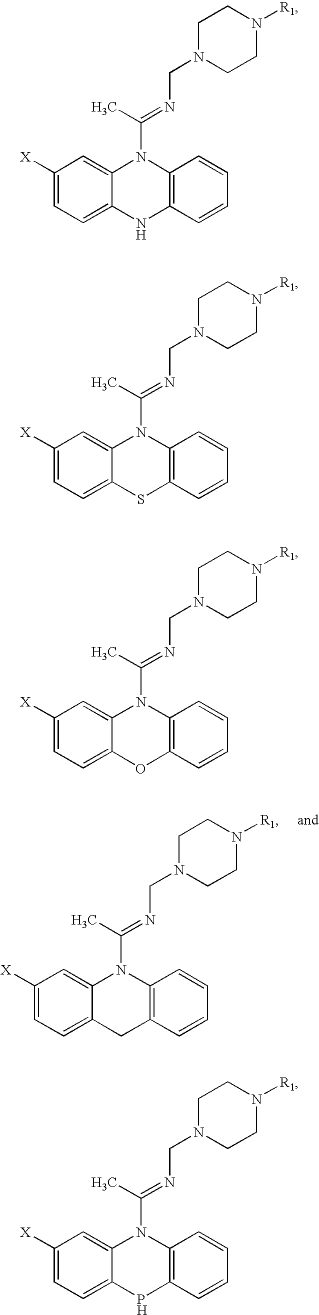

including tautomers and geometric isomers thereof and pharmaceutical derivatives thereof.

In some embodiments, the compounds have a structure selected from a group consisting of:

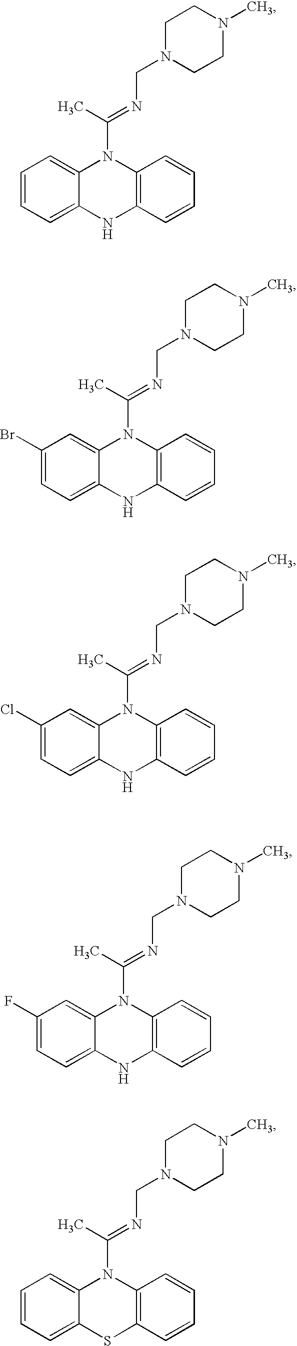

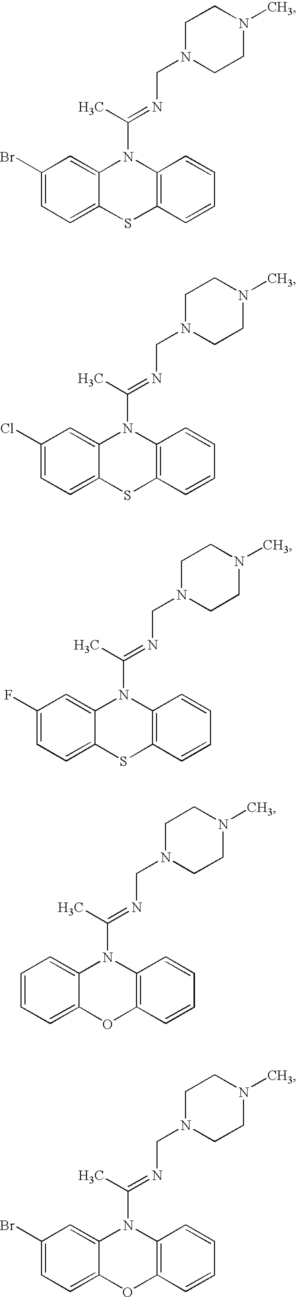

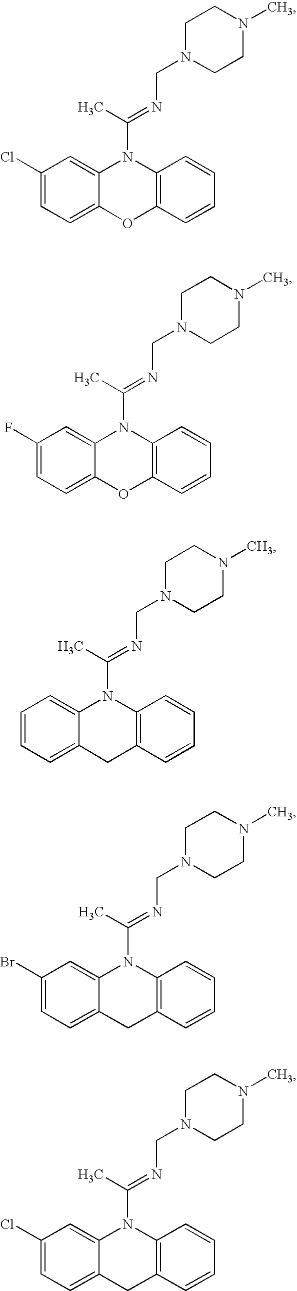

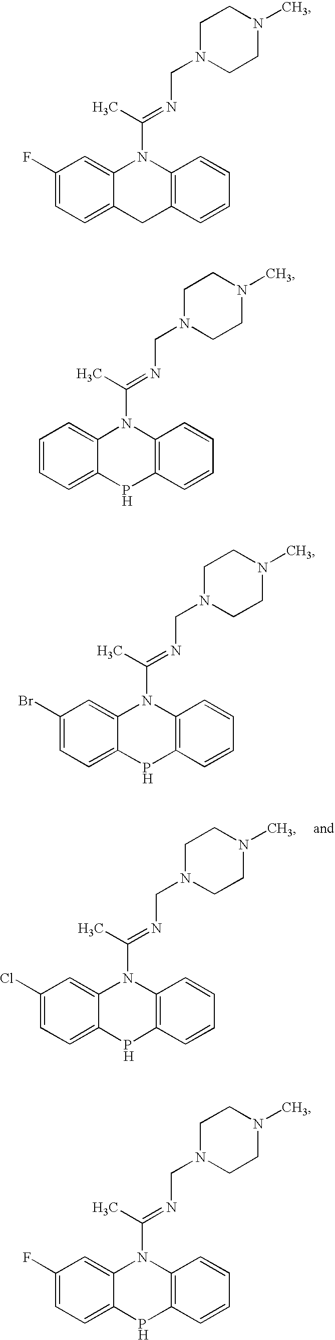

including tautomers and geometric isomers thereof and pharmaceutical derivatives thereof.

In various embodiments, compounds having structure V are disclosed herein:

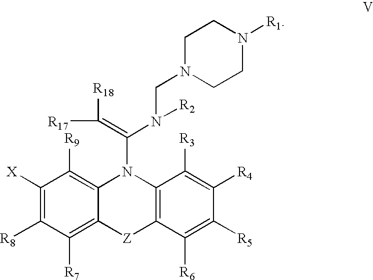

Tautomers, geometric isomers and pharmaceutical derivatives of compound V are disclosed herein. X is selected from a group consisting of H, F, Cl, and Br. Z is selected from a group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂. R₁₂ is selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₃ is selected from a group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₄ and R₁₅ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio. R₁ and R₂ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl. R₃-R₉ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₁₇ and R₁₈ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

In some embodiments, the compounds have structure VI:

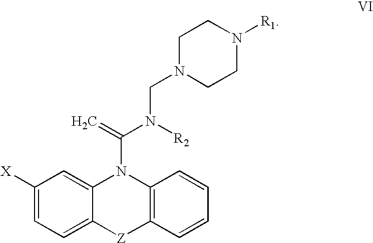

Tautomers and pharmaceutical derivatives of structure VI are disclosed herein.

In some embodiments, the compounds have a structure selected from the group consisting of:

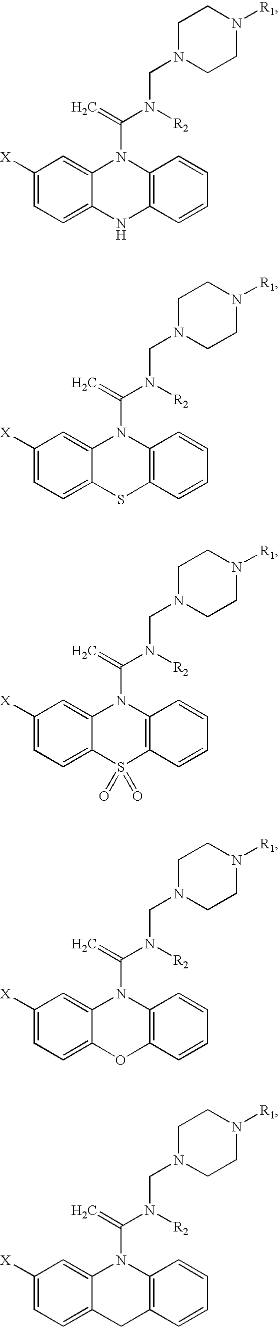

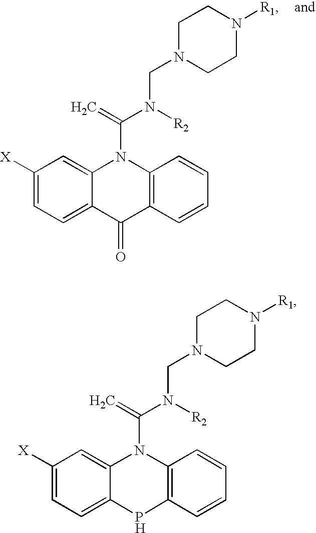

including tautomers thereof and pharmaceutical derivatives thereof.

In some embodiments, the compounds have a structure selected from the group consisting of:

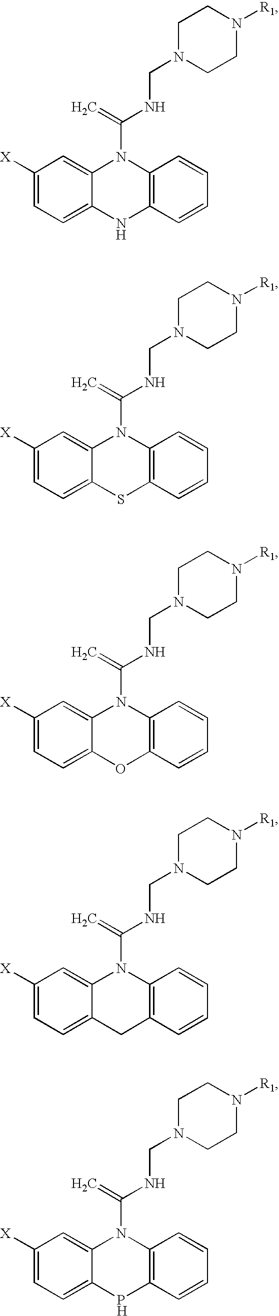

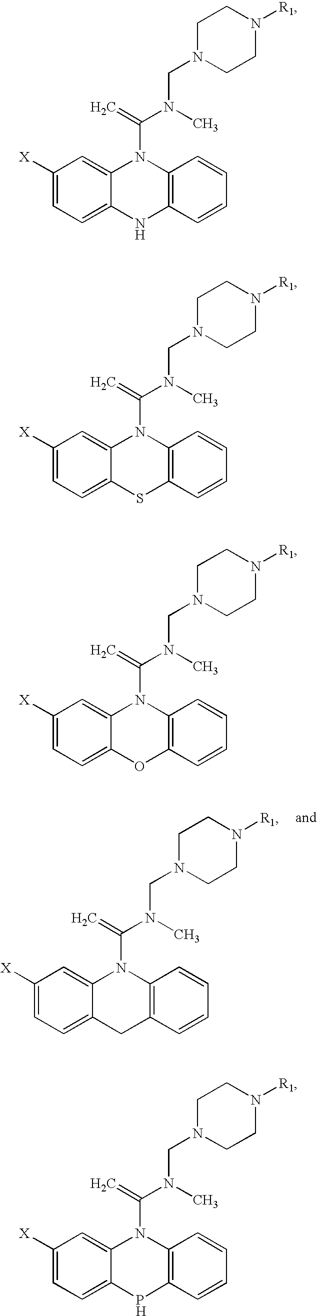

including tautomers thereof and pharmaceutical derivatives thereof.

In some embodiments, the compounds have a structure selected from the group consisting of:

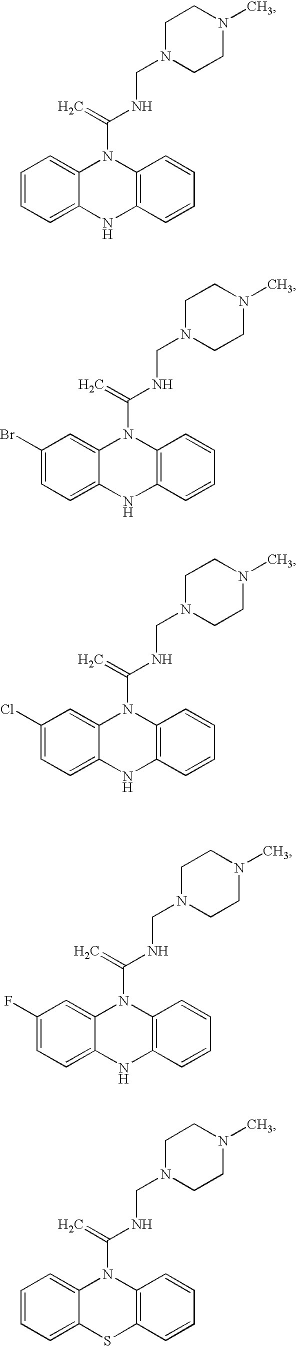

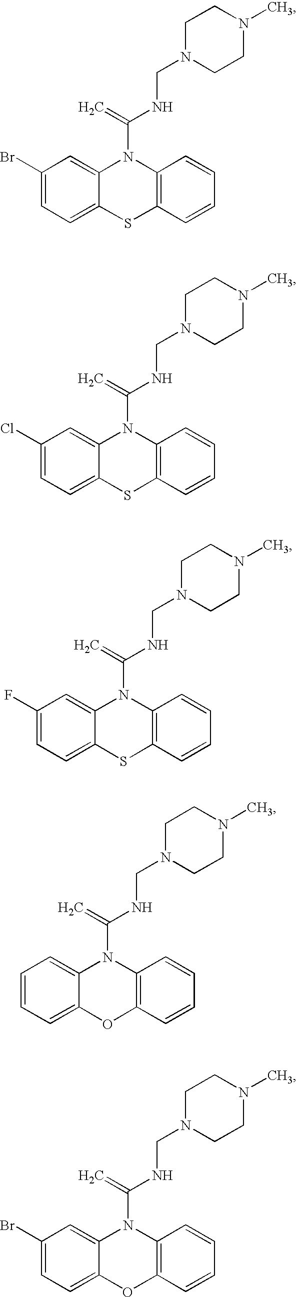

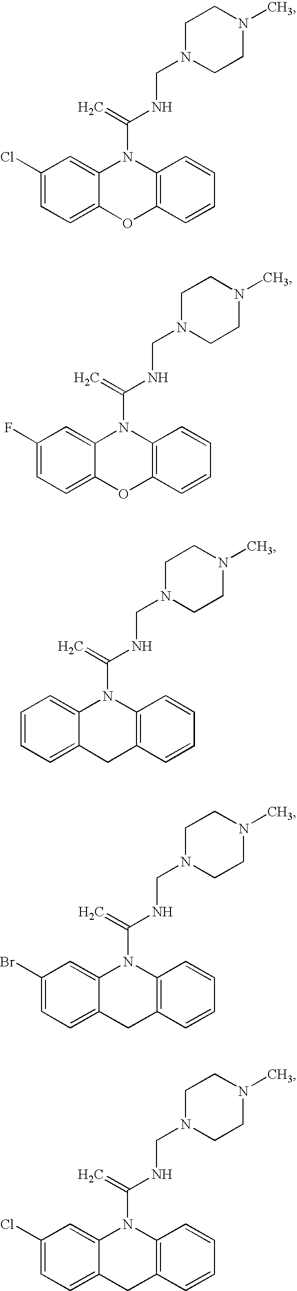

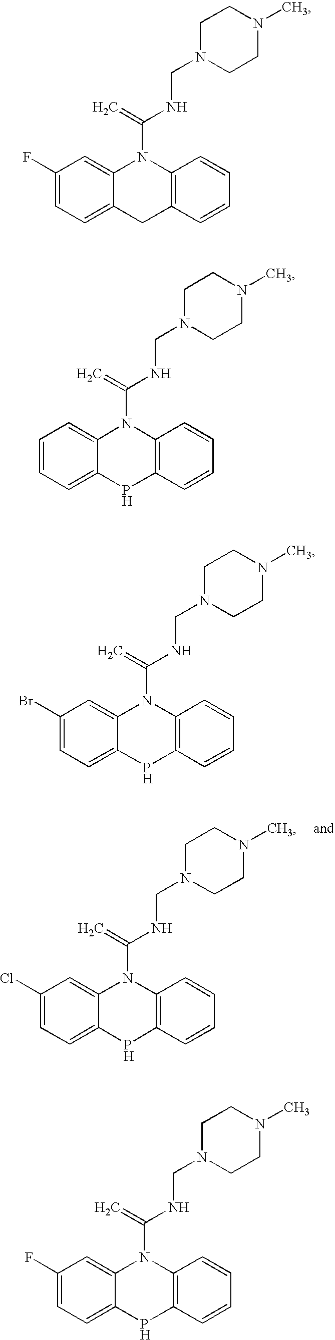

including tautomers thereof and pharmaceutical derivatives thereof

In some embodiments, the compounds have a structure selected from the group consisting of:

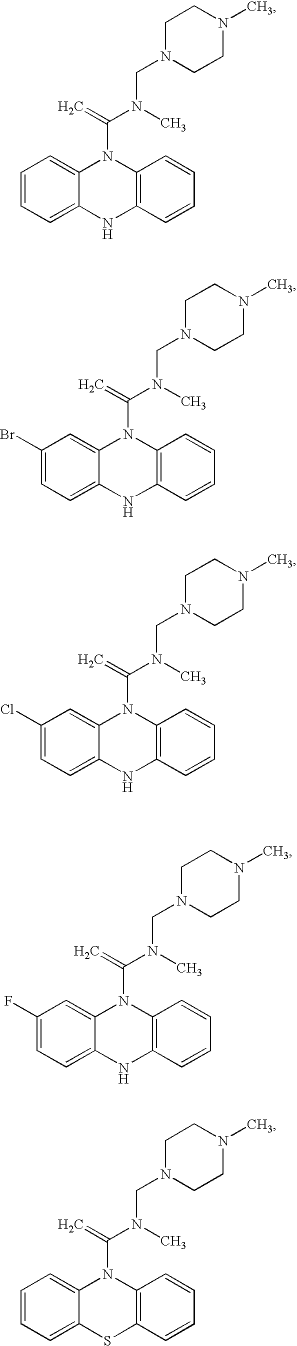

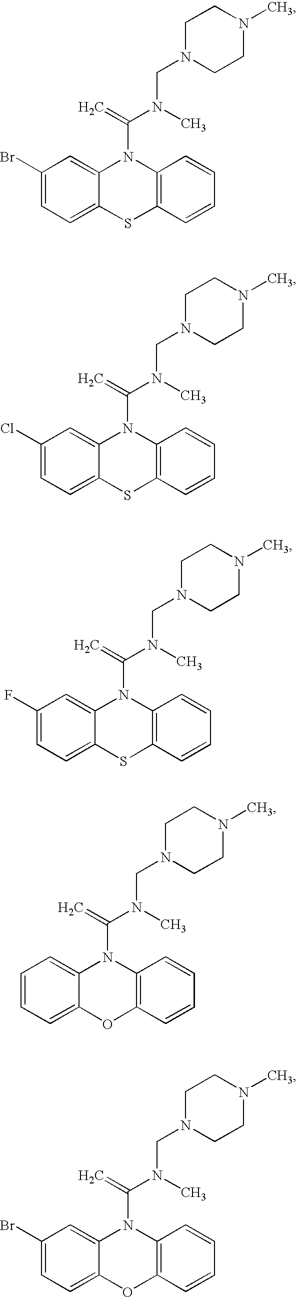

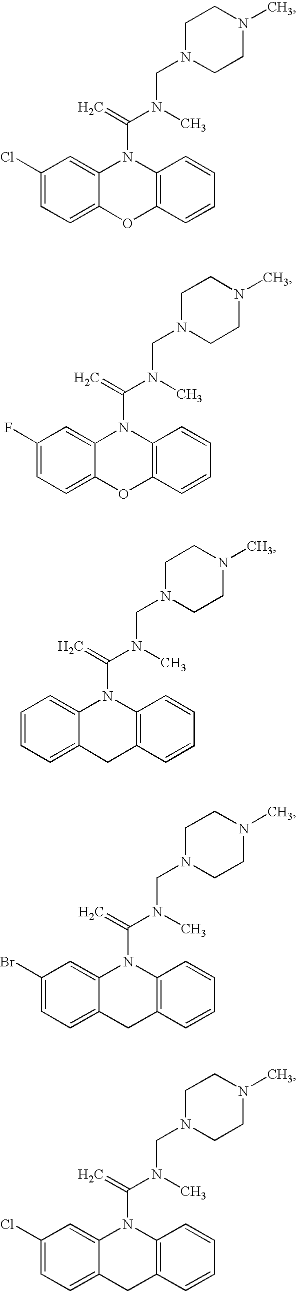

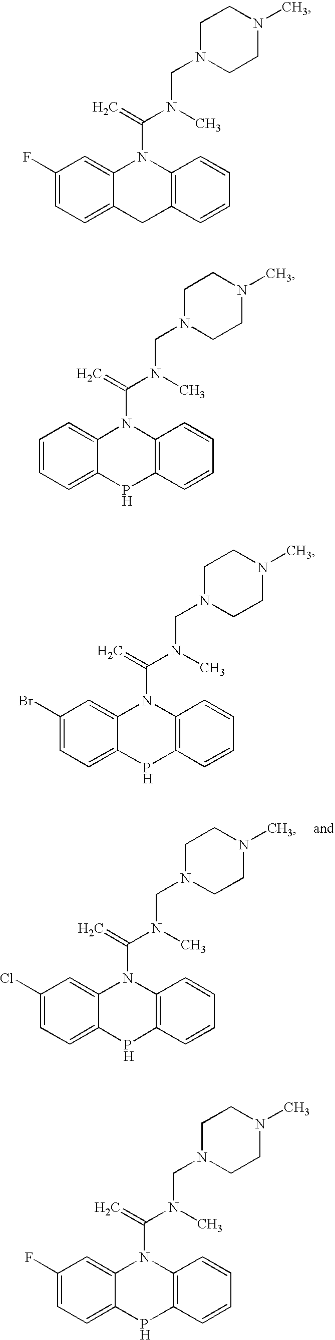

including pharmaceutical derivatives thereof.

In various embodiments, compounds having structure VII are disclosed herein:

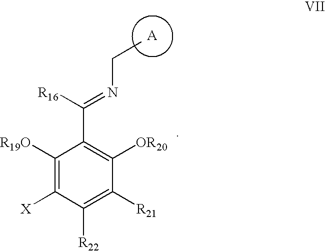

Tautomers, geometric isomers, and pharmaceutical derivatives of structure VII are disclosed herein. A is a ring selected from a group consisting of a heterocyclic ring and a heteroaromatic ring, wherein 3 to 9 carbon atoms and 1 to 4 nitrogen atoms comprise the ring. X is selected from a group consisting of H, F, Cl, and Br. R₁₆ is selected from a group consisting of alkyl, alkenyl, alkynyl, heteroalkyl, aralkyl, cycloalkyl, cycloalkylalkyl, heterocyclyl, heterocyclylalkyl, and heteroarylalkyl. R₁₉ and R₂₀ are independently selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl. R₂₁ and R₂₂ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio.

In certain embodiments, the compounds have a structure selected from the group consisting of:

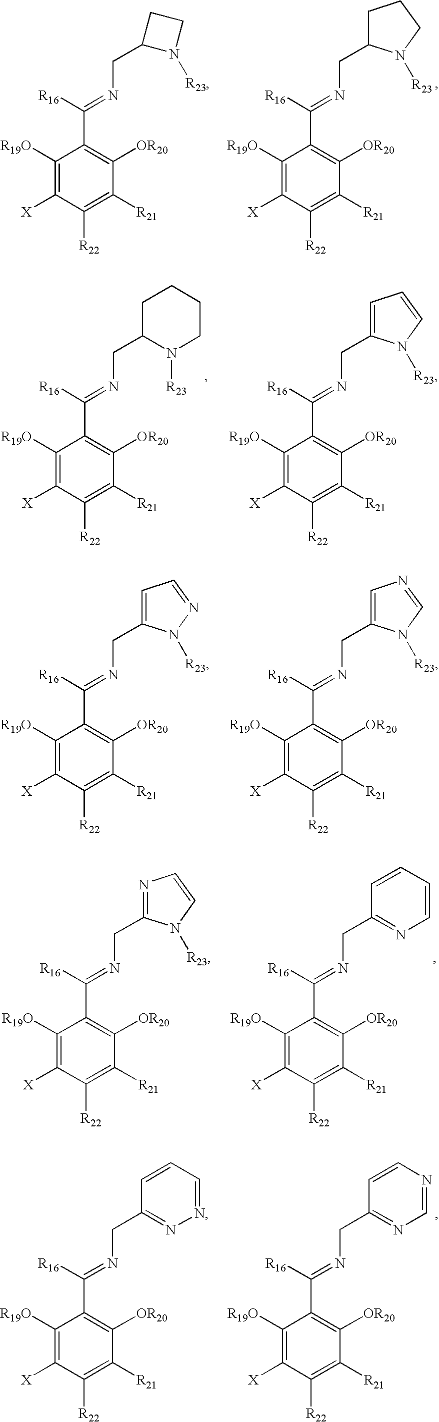

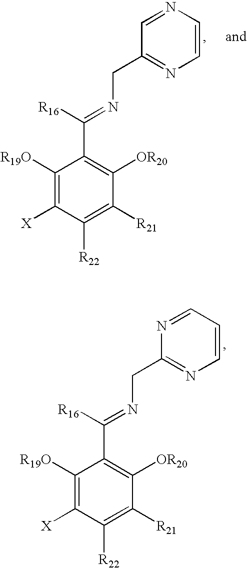

including tautomers, stereoisomers, and geometric isomers thereof and pharmaceutical derivatives thereof. R₂₃ selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

In some embodiments, the compounds have structure VIII comprising pyrrolidine:

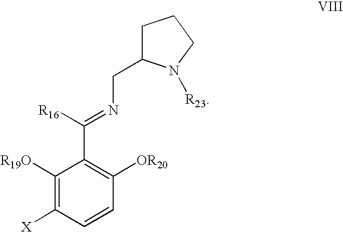

Tautomers, stereoisomers, and geometric isomers, and pharmaceutical derivatives of structure VIII are disclosed herein.

In some embodiments, the compounds have a structure selected from the group consisting of:

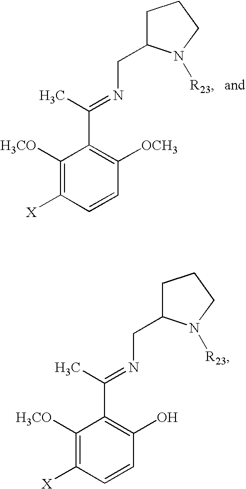

including tautomers, stereoisomers, and geometric isomers thereof and pharmaceutical derivatives thereof.

In some embodiments, the compounds have a structure selected from the group consisting of:

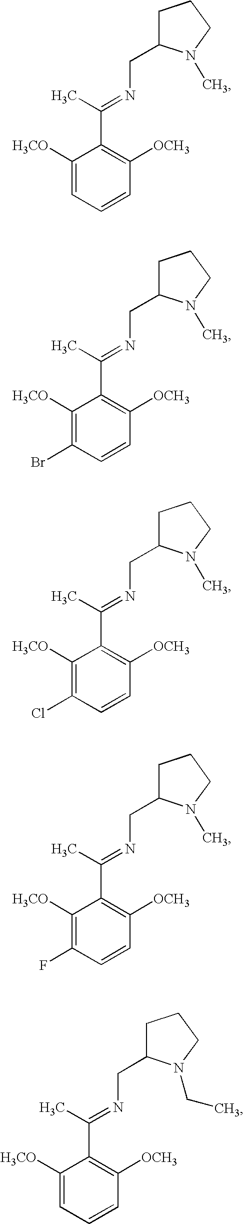

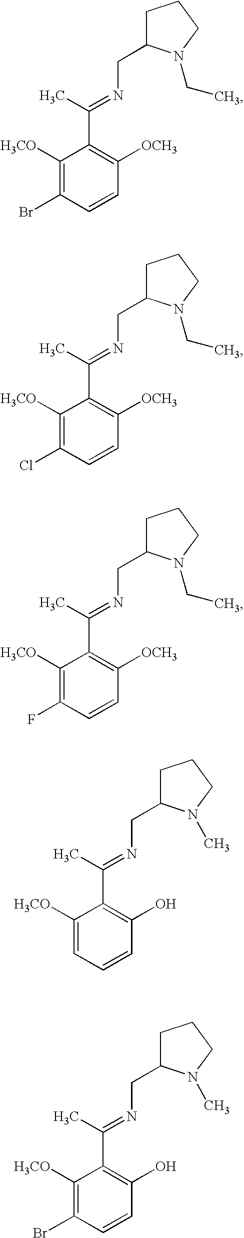

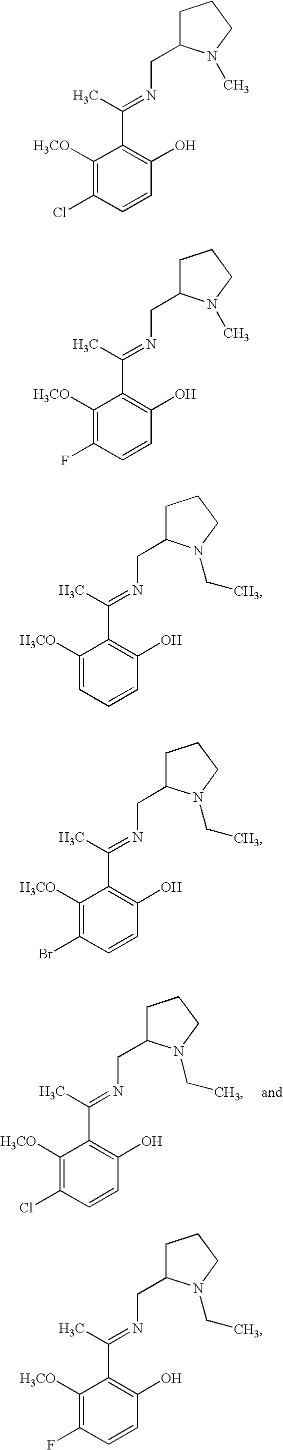

including tautomers, stereoisomers, and geometric isomers thereof and pharmaceutical derivatives thereof. In certain embodiments, the compounds comprise the R enantiomer of pyrrolidine.

In various embodiments, compounds having structure IX are disclosed herein:

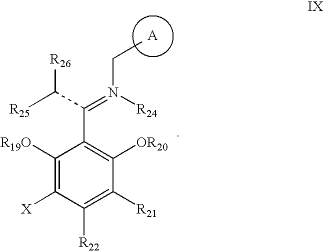

Tautomers, stereoisomers, geometric isomers, and pharmaceutical derivatives of structure IX are disclosed herein. A is a ring selected from a group consisting of a heterocyclic ring and a heteroaromatic ring, wherein 3 to 9 carbon atoms and 1 to 4 nitrogen atoms comprise the ring. The dashed bond comprises a bond selected from a group consisting of a single bond and a double bond. X is selected from a group consisting of H, F, Cl, and Br. R₁₉ and R₂₀ are independently selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, and heteroarylalkylcarbonyl. R₂₁ and R₂₂ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N-dialkyureido, N,N-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₂₄ is selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl. R₂₅ and R₂₆ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

In some embodiments, the compounds have a structure selected from the group consisting of:

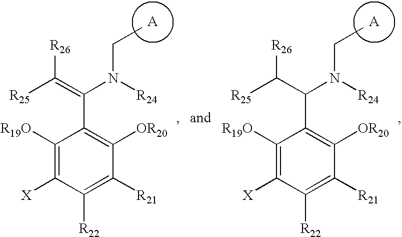

including tautomers, stereoisomers, and geometric isomers thereof and pharmaceutical derivatives thereof.

In some embodiments, the compounds have a structure selected from the group consisting of:

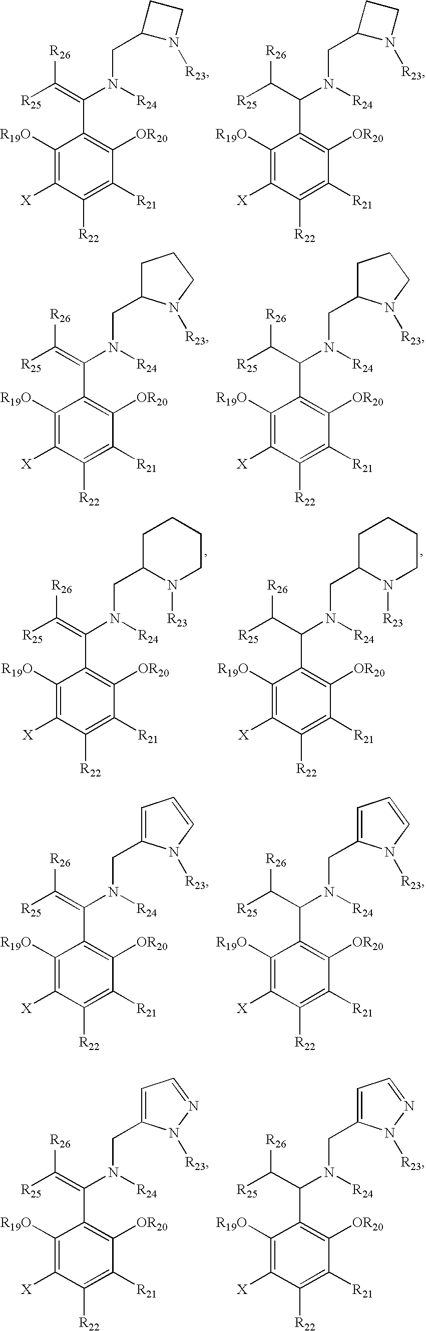

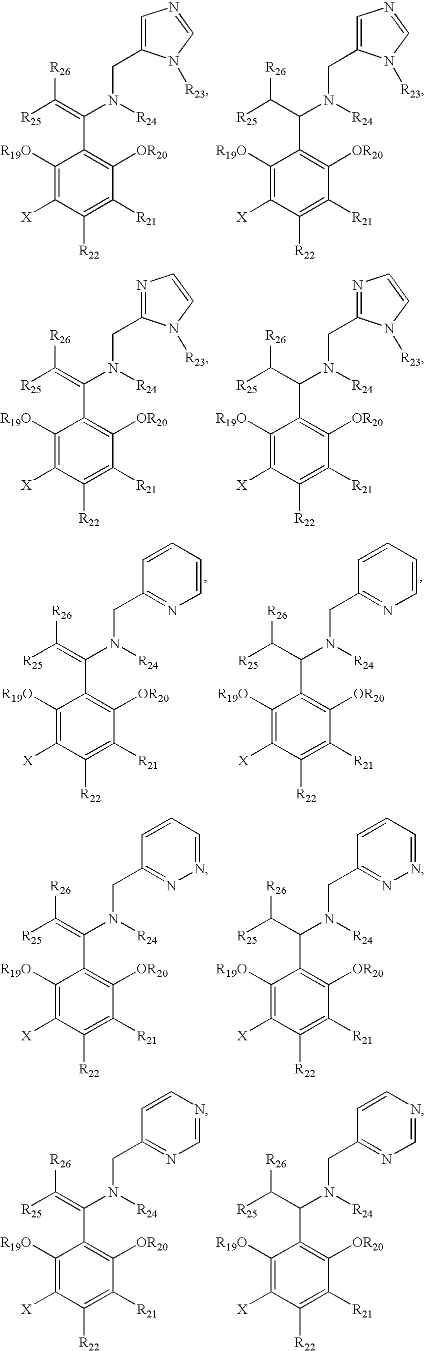

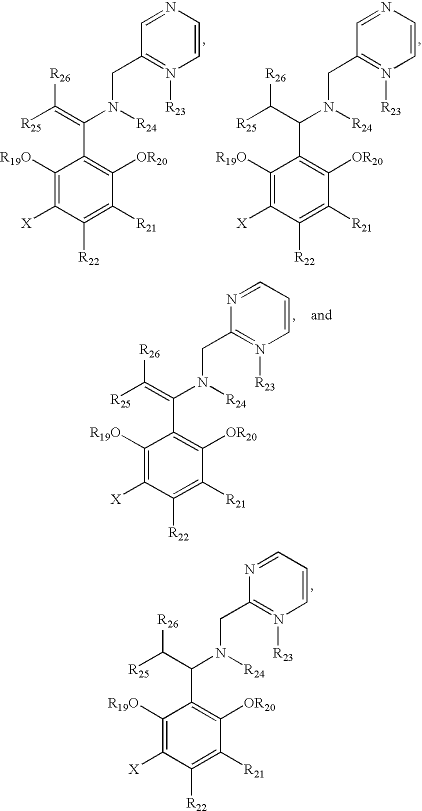

including tautomers, stereoisomers, and geometric isomers thereof and pharmaceutical derivatives thereof. R₂₃ selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cyclo alkyl carbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

In some embodiments, the compounds have a structure comprising pyrrolidine selected from the group consisting of:

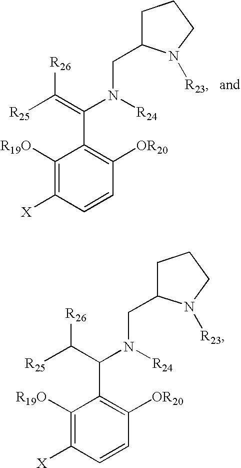

including tautomers, stereoisomers, and geometric isomers thereof and pharmaceutical derivatives thereof.

In some embodiments, the compounds have a structure selected from the group consisting of:

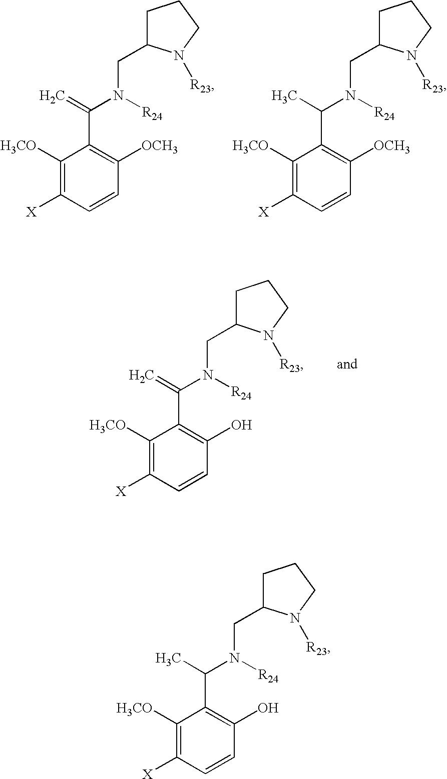

including tautomers, stereoisomers, and geometric isomers thereof, and pharmaceutical derivatives thereof.

In some embodiments, the compounds have a structure selected from the group consisting of:

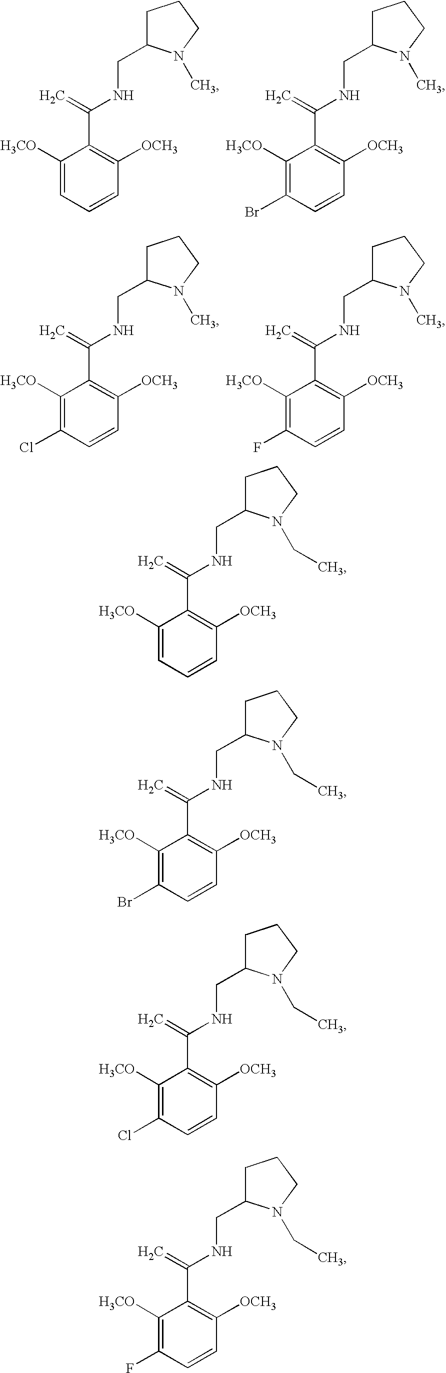

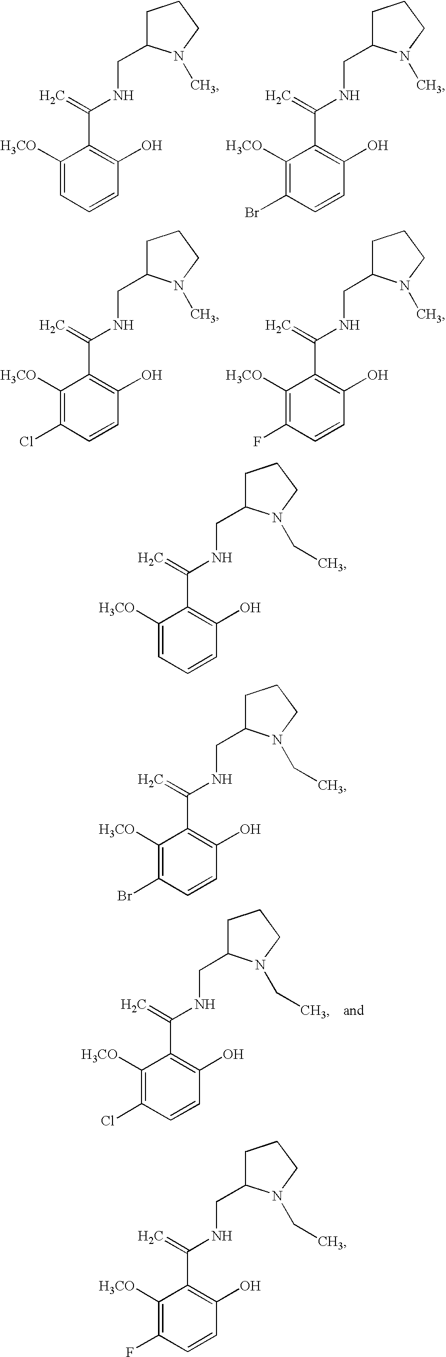

including tautomers, stereoisomers, and geometric isomers thereof and pharmaceutical derivatives thereof. In certain embodiments, the compounds comprise the R enantiomer of pyrrolidine.

In some embodiments, the compounds have a structure selected from the group consisting of:

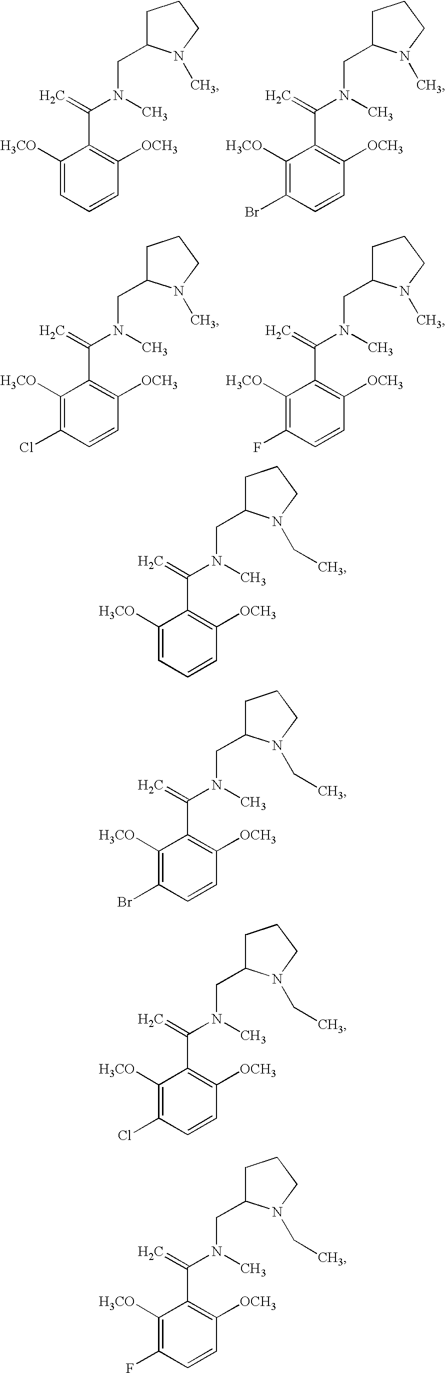

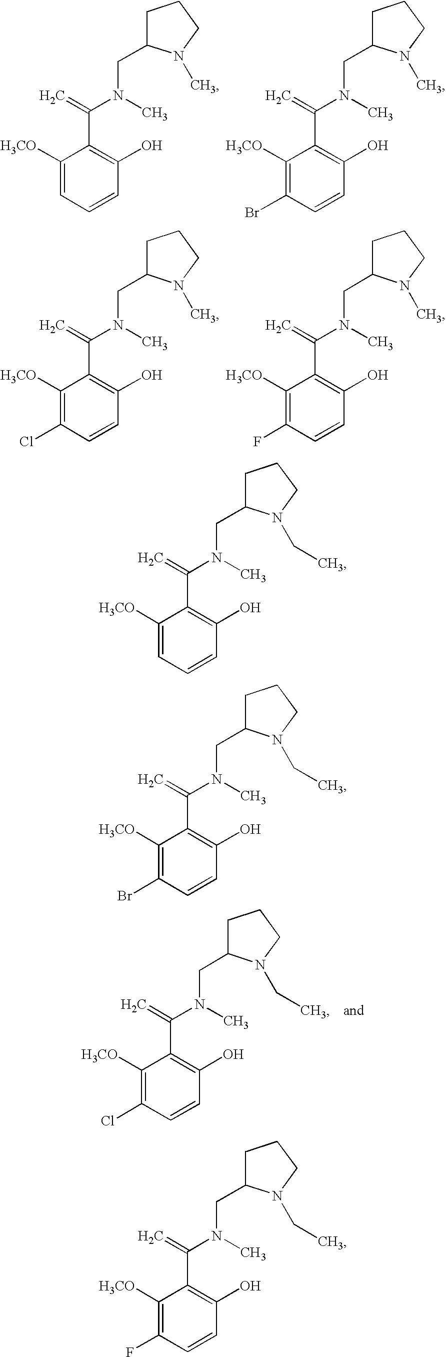

including stereoisomers and pharmaceutical derivatives thereof. In some embodiments, the compounds comprise the R enantiomer of pyrrolidine.

In some embodiments, the compounds have a structure selected from the group consisting of:

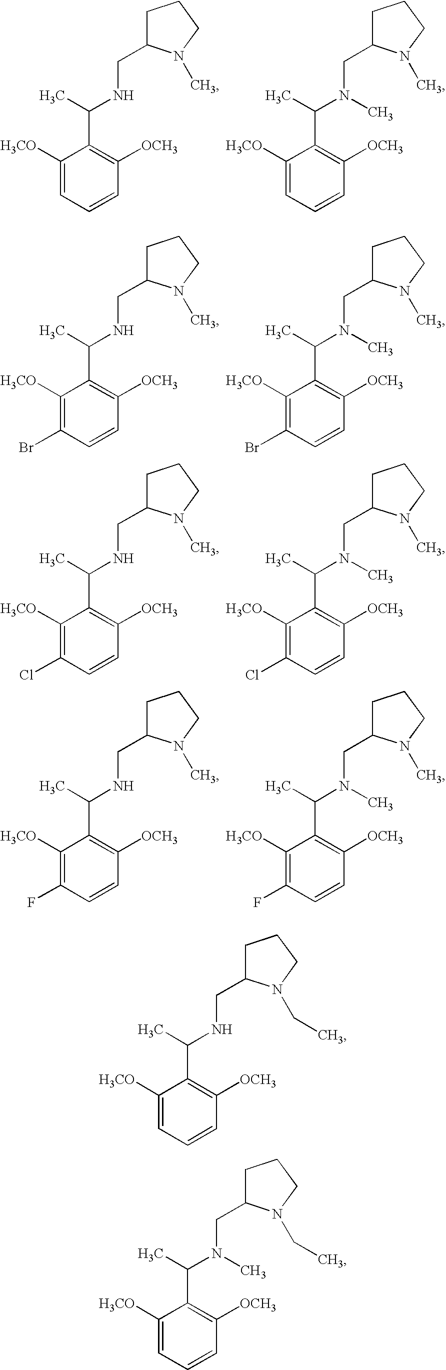

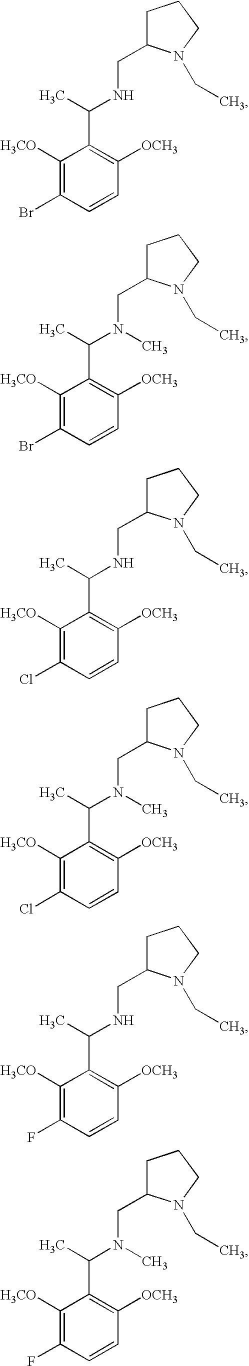

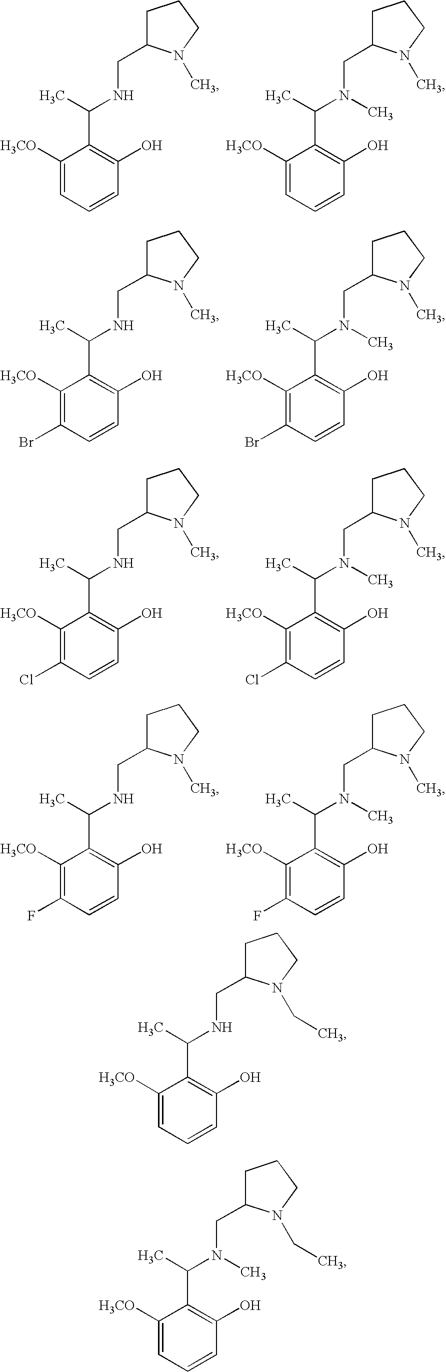

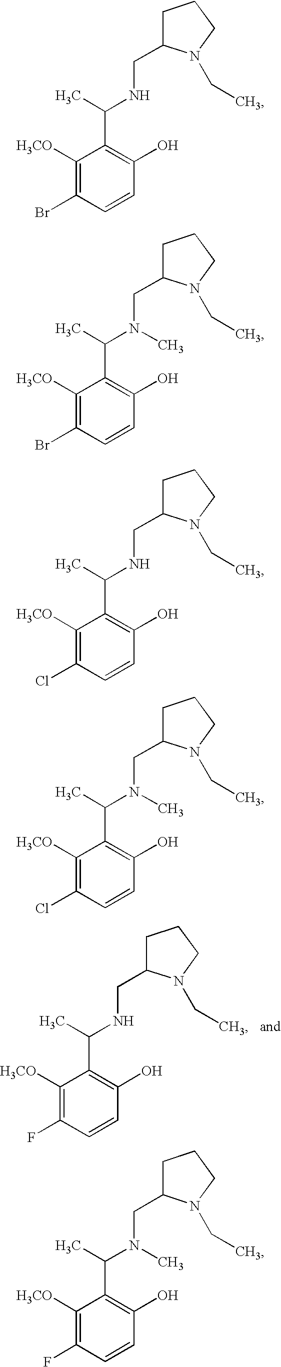

including stereoisomers and pharmaceutical derivatives thereof. In certain embodiments, the compounds comprise the R enantiomer of pyrrolidine.

In various embodiments, compounds having structure X are disclosed herein:

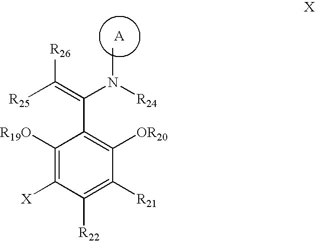

Tautomers, stereoisomers, geometric isomers and pharmaceutical derivatives of structure X are disclosed herein. A is a ring selected from a group consisting of a heterocyclic ring and a heteroaromatic ring, wherein 3 to 9 carbon atoms and 1 to 4 nitrogen atoms comprise the ring. X is selected from a group consisting of H, F, Cl, and Br. R₁₉ and R₂₀ are independently selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, and heteroarylalkylcarbonyl. R₂₁ and R₂₂ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N,N′-diarylureido, N-aryl-N′,N′-dialkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₂₄ is selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl. R₂₅ and R₂₆ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

In some embodiments, the compound has a structure selected from the group consisting of:

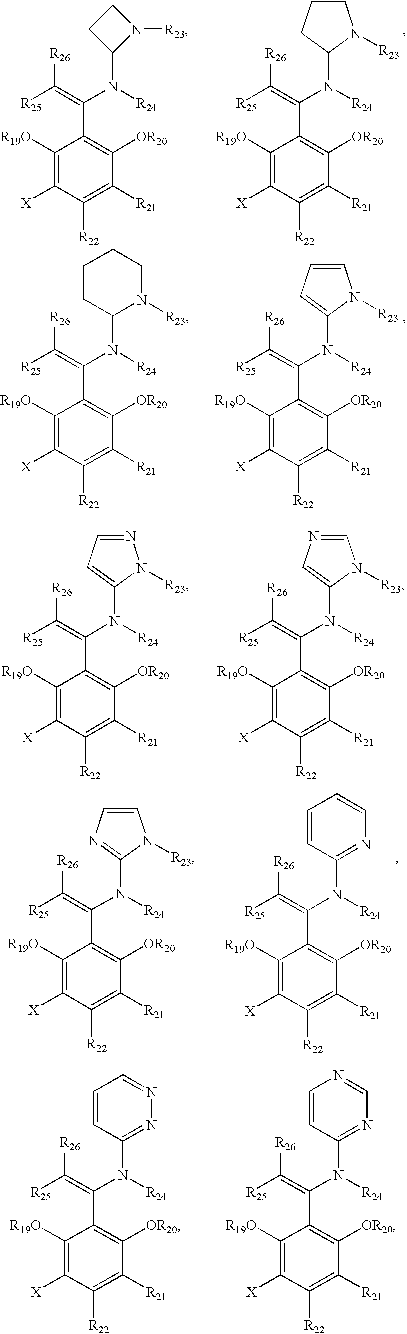

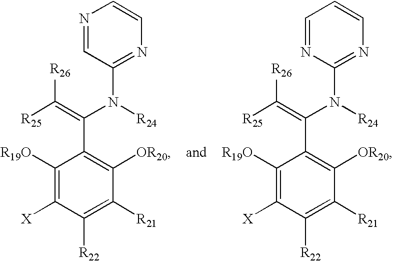

including tautomers, stereoisomers, geometric isomers, and pharmaceutical derivatives thereof. R₂₃ selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

In various embodiments, compounds having structure XI comprising pyrrolidine are disclosed herein:

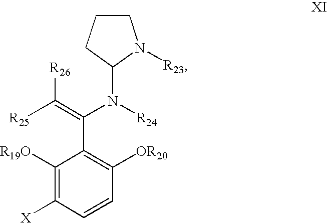

including tautomers, stereoisomers, geometric isomers, and pharmaceutical derivatives thereof.

In some embodiments, the compounds have a structure selected from the group consisting of:

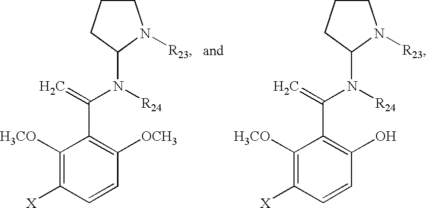

including tautomers, stereoisomers, and pharmaceutical derivatives thereof.

In some embodiments, the compounds have a structure selected from the group consisting of:

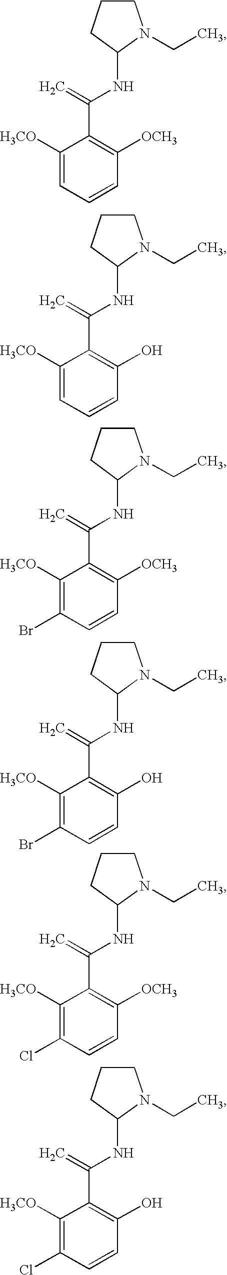

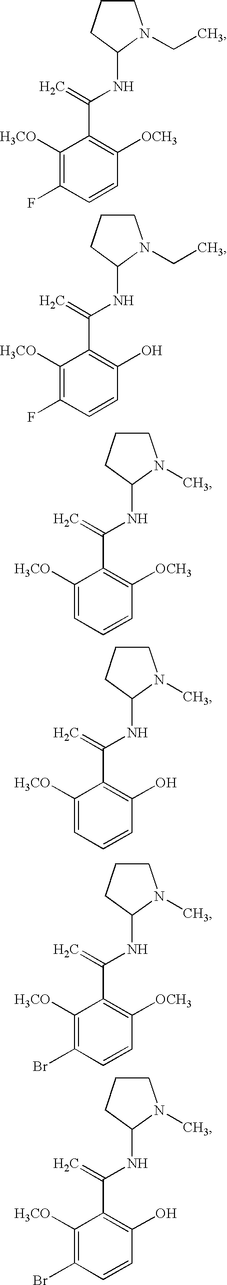

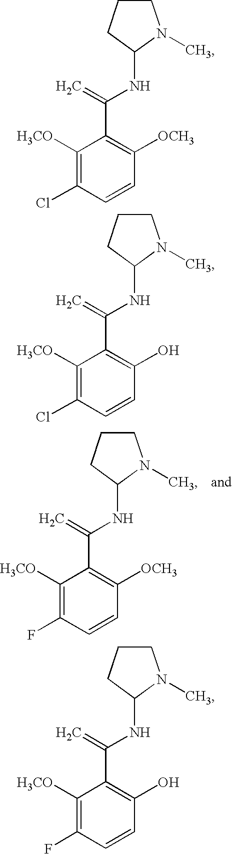

including tautomers, stereoisomers, and pharmaceutical derivatives thereof. In some embodiments, the compounds comprise the R enantiomer of pyrrolidine.

In some embodiments, the compounds have a structure selected from the group consisting of:

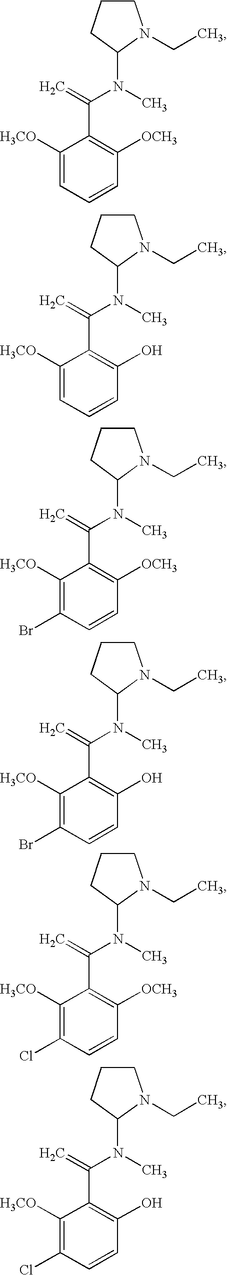

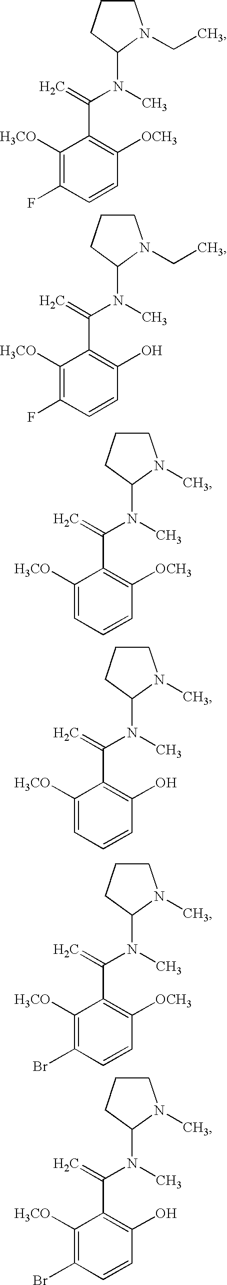

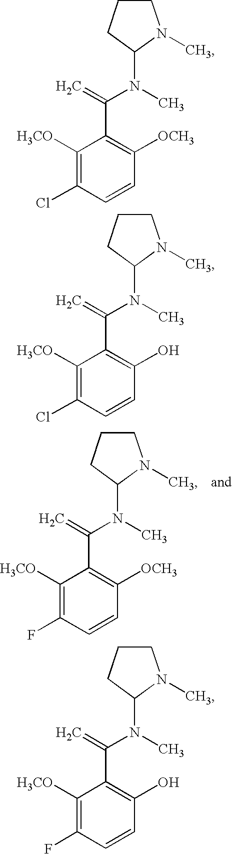

including stereoisomers and pharmaceutical derivatives thereof. In some embodiments, the compounds comprise the R enantiomer of pyrrolidine.

In various embodiments, compounds having structure XII are disclosed herein:

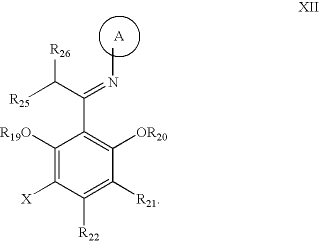

Tautomers, stereoisomers, geometric isomers, and pharmaceutical derivatives of structure XII are disclosed herein. A is a ring selected from a group consisting of a heterocyclic ring and a heteroaromatic ring, wherein 3 to 9 carbon atoms and 1 to 4 nitrogen atoms comprise the ring. X is selected from a group consisting of H, F, Cl, and Br. R₁₉ and R₂₀ are independently selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, and heteroarylalkylcarbonyl. R₂₁ and R₂₂ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, acylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₂₅ and R₂₆ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

In some embodiments, the compounds have a structure selected from the group consisting of:

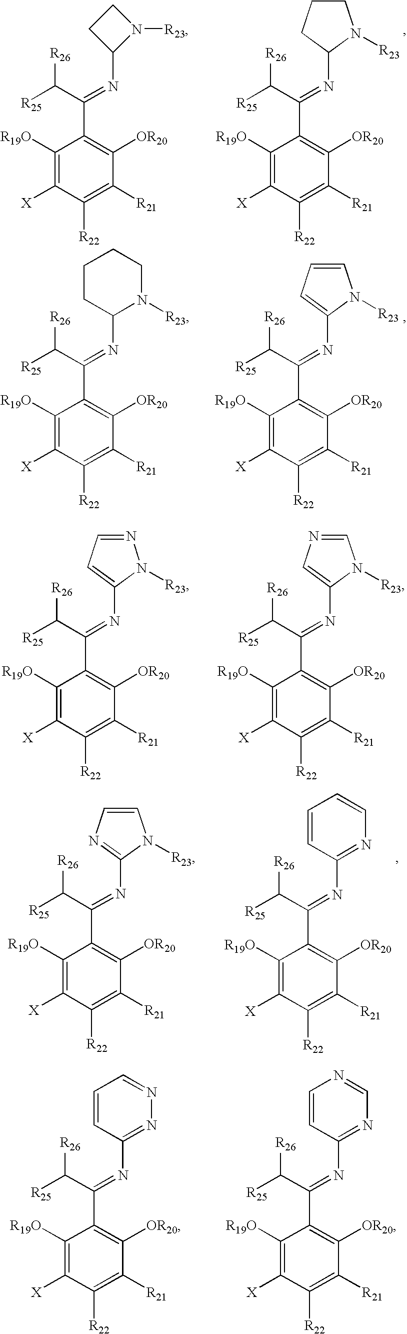

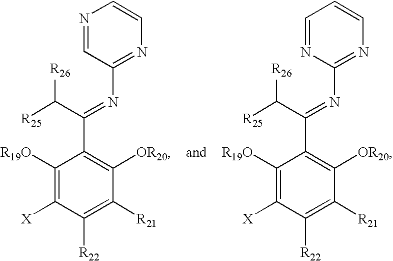

including tautomers, stereoisomers, geometric isomers, and pharmaceutical derivatives thereof. R₂₃ selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

In some embodiments, the compounds have structure XIII comprising pyrrolidine:

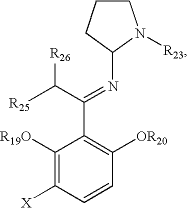

including tautomers, stereoisomers, geometric isomers, and pharmaceutical derivatives thereof.

In some embodiments, the compounds have a structure selected from the group consisting of:

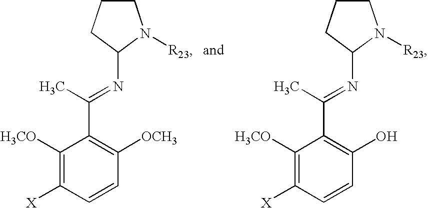

including tautomers, stereoisomers, geometric isomers, and pharmaceutical derivatives thereof.

In some embodiments, the compounds have a structure selected from the group consisting of:

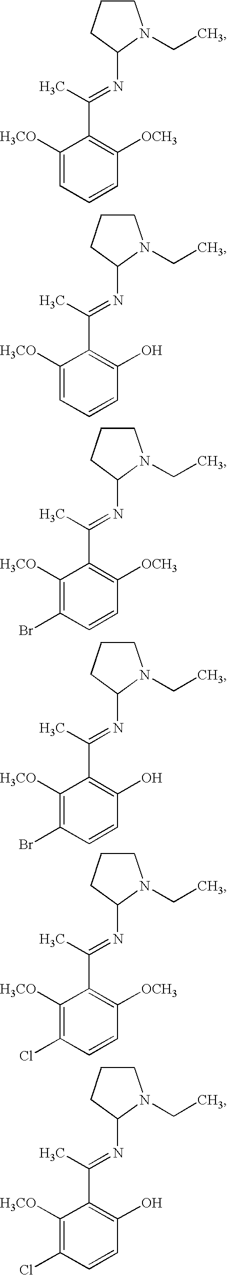

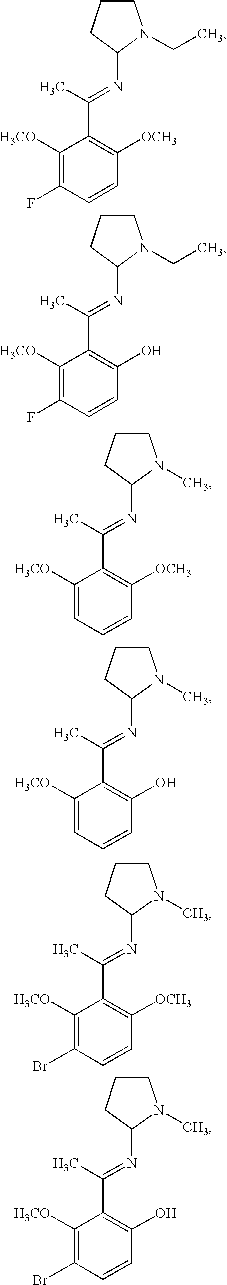

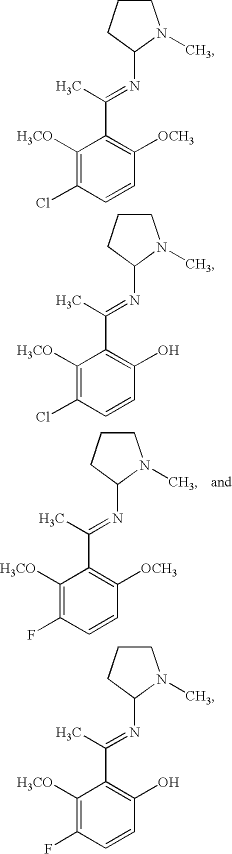

including tautomers, stereoisomers, geometric isomers, and pharmaceutical derivatives thereof. In some embodiments, the compounds comprise the R enantiomer of pyrrolidine.

In various embodiments, compounds having structure XIV are disclosed herein:

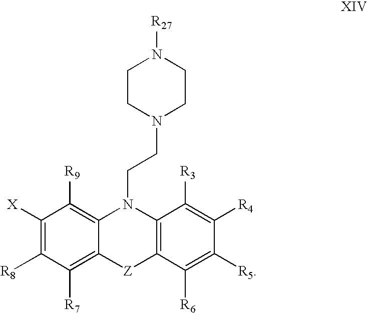

Pharmaceutical derivatives of structure XIV are disclosed herein. X is selected from a group consisting of H, F, Cl, and Br. Z is selected from a group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂. R₁₂ is selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₃ is selected from a group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₄ and R₁₅ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenyl carbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio. R₃-R₉ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′N-dialkylureido, N,N-diaryl-N-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₂₇ is selected from a group consisting of H, C1-C4 alkyl, and phenyl, wherein the C1-C4 alkyl comprises methyl, ethyl, propyl, isopropyl, butyl, s-butyl, t-butyl, and isobutyl. The selection of X, Z, R₃-R₉ and R₂₇ is conducted with the proviso that none of the following conditions are met:

i) X is H, Z is S, R₂₇ is selected from a group consisting of H, methyl, ethyl, propyl, butyl, and phenyl, and R₃-R₉ are all H;

ii) X is H, Z is S, R₄ is methoxy, and R₃, R₅-R₉ and R₂₇ are all H;

iii) X is H, Z is S, R₃ and R₇-R₉ are all H, any R₄-R₆ that are not Cl, methyl, ethyl, mercaptomethyl, methylcarbonyl, ethylcarbonyl, propylcarbonyl, methoxycarbonyl, or trifluoromethyl are all H, R₂₇ is selected from a group consisting of H, methyl, ethyl, and butyl, and at least one of R₄-R₆ is selected from a group consisting of Cl, methyl, ethyl, mercaptomethyl, methylcarbonyl, ethylcarbonyl, propylcarbonyl, methoxycarbonyl, and trifluoromethyl;

iv) X is H, Z is SO₂, R₃-R₉ are all H, and R₂₇ is selected from a group consisting of H, methyl, ethyl, isobutyl, and t-butyl;

v) X is H, Z is O, R₃-R₉ are all H, and R₂₇ is selected from a group consisting of H, methyl, and ethyl;

vi) X is H, Z is O, R₃ and R₅-R₉ are all H, R₂₇ is methyl, and R₄ is ethyl;

vii) X is Cl, Z is S, R₃-R₉ are all H, and R₂₇ is selected from a group consisting of H, methyl, ethyl, and butyl;

viii) X is Cl, Z is S, R₃-R₆, R₈ and R₉ are all H, R₂₇ is methyl, and R₇ is Cl; and

ix) X is H, Z is C═O, R₃-R₉ are all H, and R₂₇ is selected from a group consisting of methyl and propyl.

In certain embodiments of structure XIV, X is selected from a group consisting of F, Cl, and Br, and Z is selected from a group consisting of NR₁₂, N(C═O)R₁₂, NNH(═O)R₁₃, NSO₂R₁₃, O, CR₁₄R₁₅, C═O, C═CR₁₄R₁₅, and PR₁₂.

In some embodiments, the compounds have structure XV:

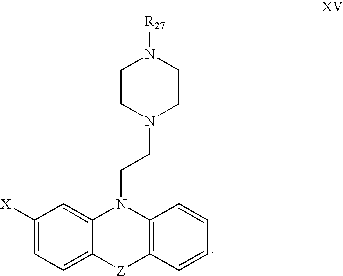

Pharmaceutical derivatives of structure XV are disclosed herein.

In some embodiments, the compounds have a structure selected from the group consisting of:

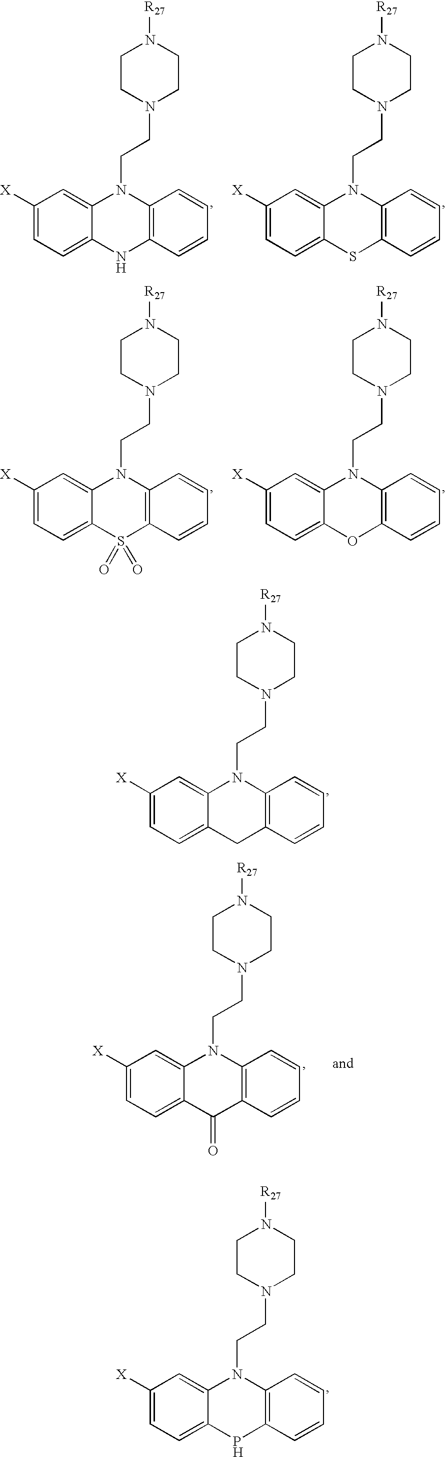

including pharmaceutical derivatives thereof.

In some embodiments, the compounds have a structure selected from a group consisting of:

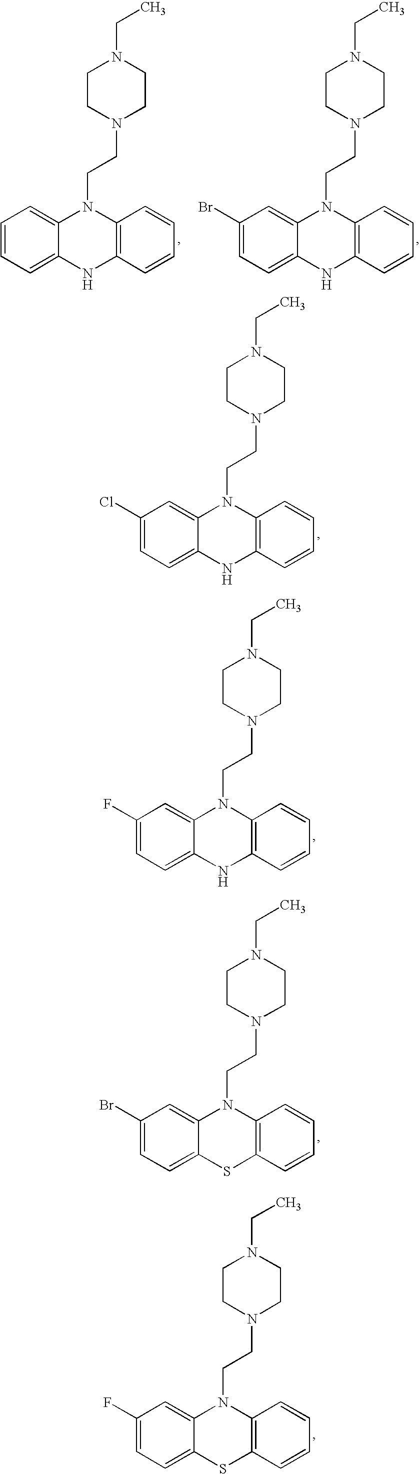

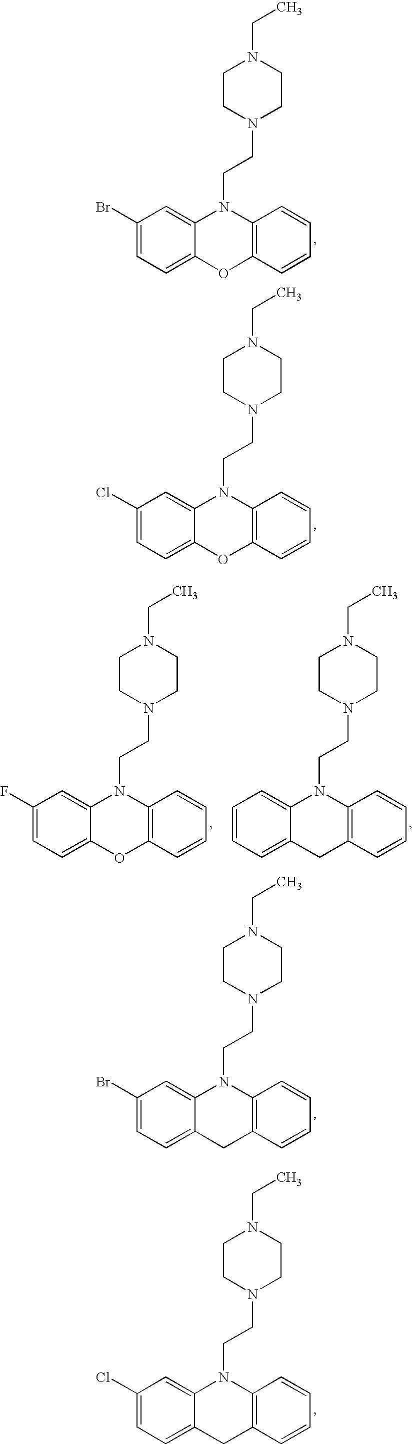

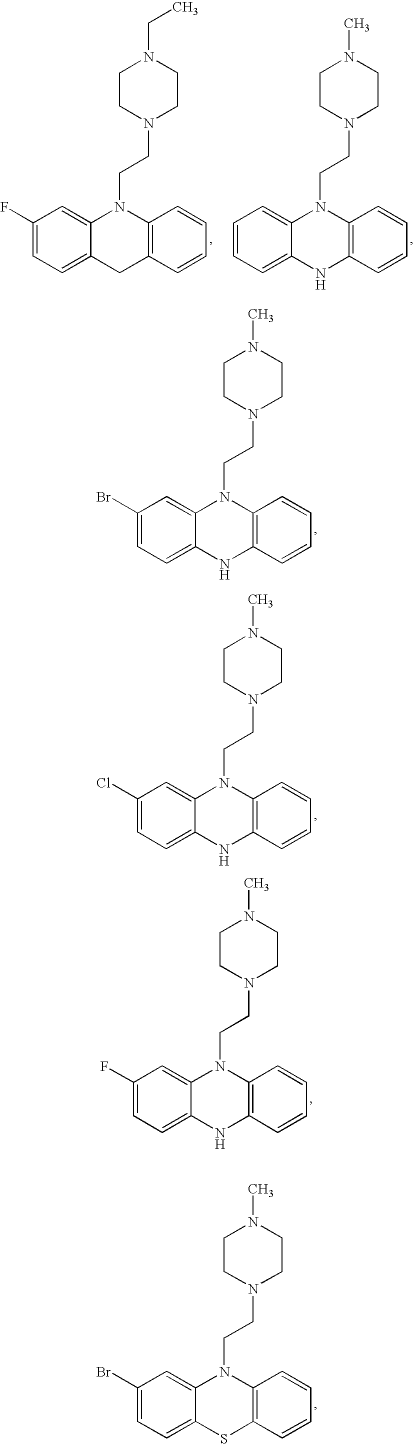

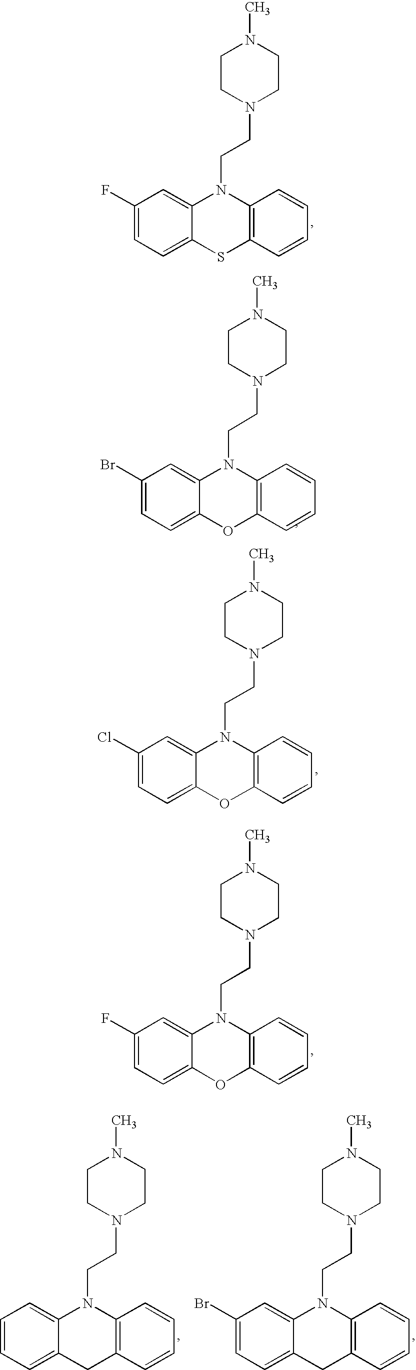

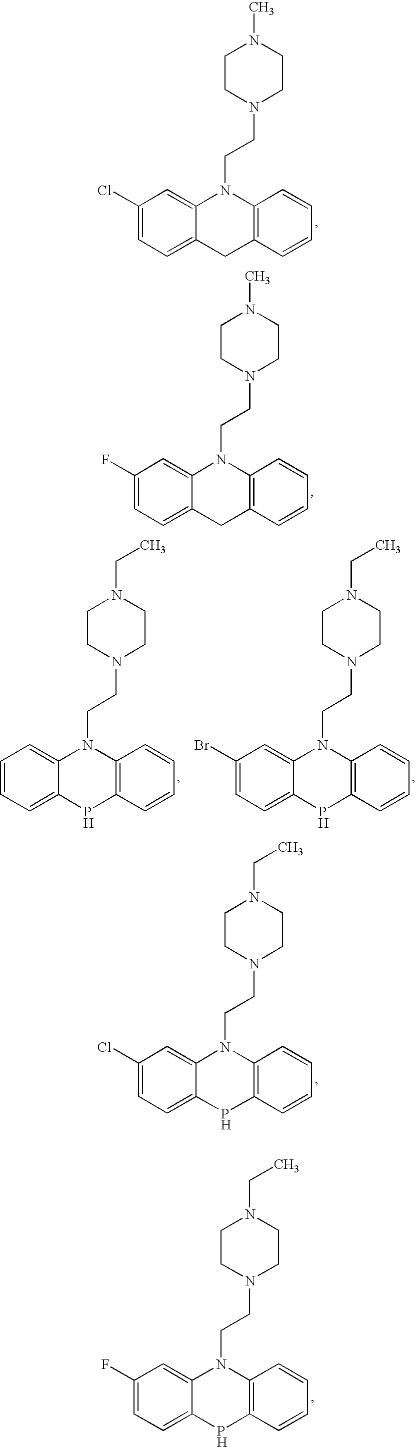

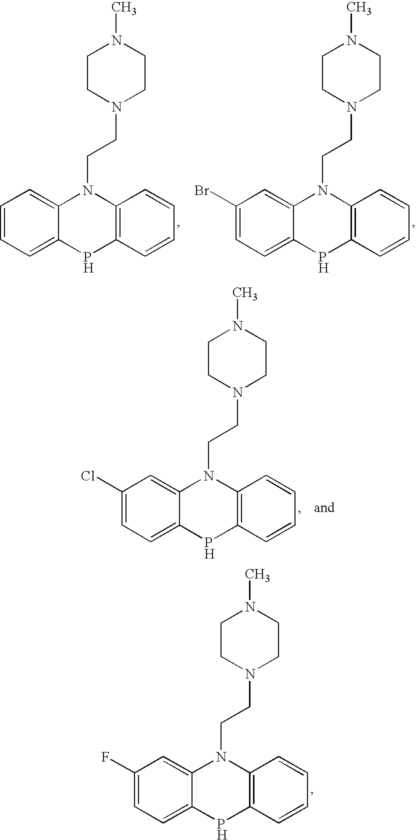

including pharmaceutical derivatives thereof.

In some embodiments, compounds having structure XVI are disclosed herein:

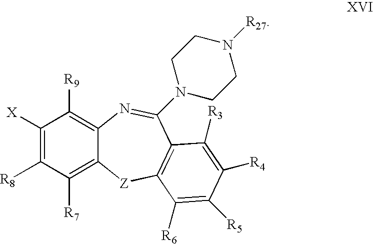

Pharmaceutical derivatives of structure XVI are disclosed herein. X is selected from a group consisting of F and Br. Z is selected from a group consisting of S, SO₂, O, CR₁₄R₁₅, C═O, C═CR₁₄R₁₅, and PR₁₂. R₁₂ is selected from a group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl. R₁₄ and R₁₅ are independently selected from a group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio. R₃-R₉ are independently selected from a group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio. R₂₇ is selected from a group consisting of H, C1-C4 alkyl, and phenyl, wherein the C1-C4 alkyl comprises methyl, ethyl, propyl, isopropyl, butyl, s-butyl, t-butyl, and isobutyl. The selection of X, Z, R₃-R₉ and R₂₇ is conducted with the proviso that none of the following conditions are met:

i) X is F, Z is O, R₃ and R₅-R₉ are all H, R₂₇ is selected from a group consisting of H, methyl, ethyl, propyl, and R₄ is selected from a group consisting of H, methyl, mercaptomethyl, bromo, amino, and nitro;

ii) X is F, Z is O, R₃ and R₅-R₉ are all H, R₂₇ is methyl, and R₄ is nitro;

iii) X is F, Z is O, R₃-R₅, R₇-R₉ and R₂₇ are all H, and R₆ is methyl;

iv) X is F, Z is O, R₃-R₆ are all H, R₂₇ is selected from a group consisting of methyl and phenyl, and R₇-R₉ are all F;

v) X is F, Z is S, R₃ and R₅-R₉ are all H, R₂₇ is selected from a group consisting of methyl, ethyl, and propyl, and R₄ is selected from a group consisting of H, mercaptomethyl, chloro, and bromo;

vi) X is Br, Z is O, R₃ and R₅-R₉ are all H, R₂₇ is selected from a group consisting of H, methyl, ethyl, and propyl, and R₄ is selected from a group consisting of H, mercaptomethyl, bromo, and trifluoromethyl;

vii) X is Br, Z is S, R₃ and R₅-R₉ are all H, R₂₇ is selected from a group consisting of methyl and ethyl, and R₄ is mercaptomethyl;

viii) X is Br, Z is O, R₃, R₄, R₆-R₉, and R₂₇ are all H, and R₅ is methoxy;

ix) X is Br, Z is O, R₃-R₅, R₇-R₉, and R₂₇ are all H, and R₆ is methyl;

x) X is F, Z is CR₁₄R₁₅, R₃-R₇, R₉, R₁₄, and R₁₅ are all H, R₂₇ is selected from a group consisting of H, methyl, isopropyl, and t-butyl, and R₈ is selected from a group consisting of H, hydroxy, methyl, fluoro, chloro, and sulfonyloxy; and

xi) X is F, Z is C═CR₁₄R₁₅, R₃-R₉, R₁₄, and R₁₅ are all H, and R₂₇ is selected from a group consisting of H and methyl.

In some embodiments, the compounds have structure XVII:

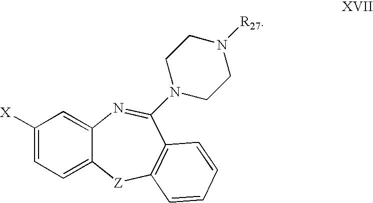

Pharmaceutical derivatives of structure XVII are disclosed herein.

In some embodiments, the compounds have a structure selected from the group consisting of:

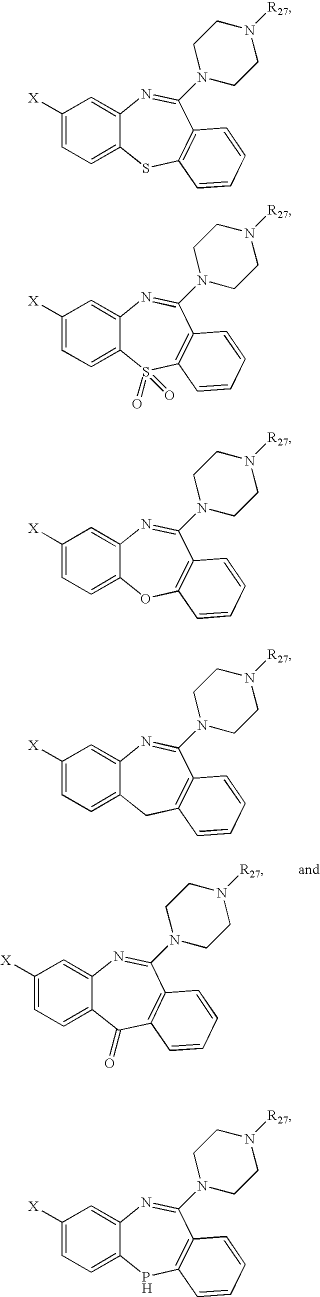

including pharmaceutical derivatives thereof.

In some embodiments, the compounds have a structure selected from the group consisting of:

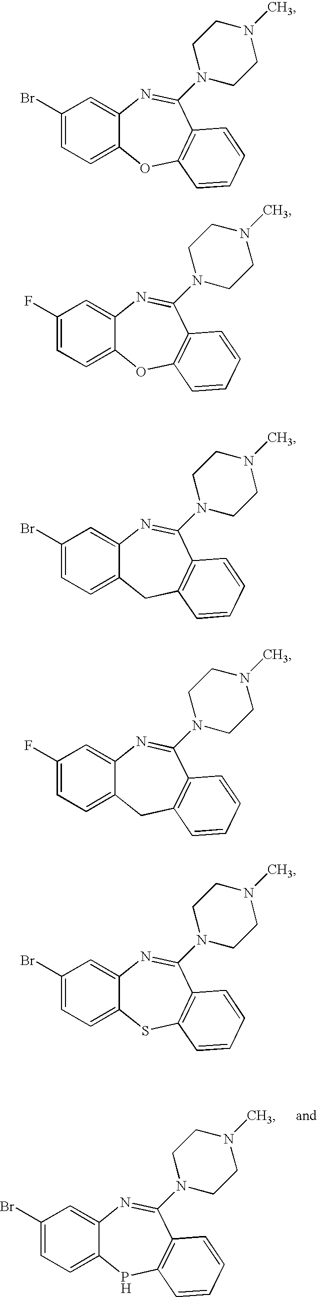

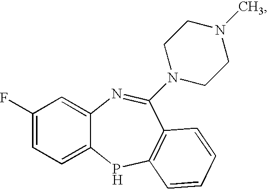

including pharmaceutical derivatives thereof.

In general, in various embodiments, any of the compounds disclosed herein, pharmaceutical derivatives thereof, and pharmaceutical compositions thereof may be used in methods for treating a psychotic condition. In some embodiments, the psychotic condition is schizophrenia.

In various embodiments, methods for treating a psychotic condition comprising administering a compound having structure I, a pharmaceutical derivative thereof, or a pharmaceutical composition thereof are disclosed.

In various embodiments, methods for treating a psychotic condition comprising administering a compound having structure III, a pharmaceutical derivative thereof, or a pharmaceutical composition thereof are disclosed.

In various embodiments, methods for treating a psychotic condition comprising administering a compound having structure V, a pharmaceutical derivative thereof, or a pharmaceutical composition thereof are disclosed.

In various embodiments, methods for treating a psychotic condition comprising administering a compound having structure VII, a pharmaceutical derivative thereof, or a pharmaceutical composition thereof are disclosed.

In various embodiments, methods for treating a psychotic condition comprising administering a compound having structure IX, a pharmaceutical derivative thereof, or a pharmaceutical composition thereof are disclosed.

In various embodiments, methods for treating a psychotic condition comprising administering a compound having structure X, a pharmaceutical derivative thereof, or a pharmaceutical composition thereof are disclosed.

In various embodiments, methods for treating a psychotic condition comprising administering a compound having structure XII, a pharmaceutical derivative thereof, or a pharmaceutical composition thereof are disclosed.

In various embodiments, methods for treating a psychotic condition comprising administering a compound having structure XIV, a pharmaceutical derivative thereof, or a pharmaceutical composition thereof are disclosed.

In various embodiments, methods for treating a psychotic condition comprising administering a compound having structure XVI, a pharmaceutical derivative thereof, or a pharmaceutical composition thereof are disclosed.

In general, in various embodiments, any of the compounds disclosed herein or a pharmaceutical derivative thereof, may be made into pharmaceutical compositions. In some embodiments, the pharmaceutical compositions are used for treating schizophrenia.

In various embodiments, pharmaceutical compositions comprising a compound having structure I or a pharmaceutical derivative thereof are disclosed.

In various embodiments, pharmaceutical compositions comprising a compound having structure III or a pharmaceutical derivative thereof are disclosed.

In various embodiments, pharmaceutical compositions comprising a compound having structure V or a pharmaceutical derivative thereof are disclosed.

In various embodiments, pharmaceutical compositions comprising a compound having structure VII or a pharmaceutical derivative thereof are disclosed.

In various embodiments, pharmaceutical compositions comprising a compound having structure IX or a pharmaceutical derivative thereof are disclosed.

In various embodiments, pharmaceutical compositions comprising a compound having structure X or a pharmaceutical derivative thereof are disclosed.

In various embodiments, pharmaceutical compositions comprising a compound having structure XII or a pharmaceutical derivative thereof are disclosed.

In various embodiments, pharmaceutical compositions comprising a compound having structure XIV or a pharmaceutical derivative thereof are disclosed.

In various embodiments, pharmaceutical compositions comprising a compound having structure XVI or a pharmaceutical derivative thereof are disclosed.

### Pharmaceutical Formulations of the Compounds

Pharmaceutical compositions contain at least one of the compounds provided herein. The compounds are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Formulation of compounds into pharmaceutical compositions uses techniques and procedures well known in the art.

In the compositions, effective concentrations of one or more compounds or pharmaceutically acceptable derivatives is (are) mixed with a suitable pharmaceutical carrier or vehicle. The compounds may comprise a pharmaceutical derivative prepared prior to formulation, such as the corresponding salts, esters, enol ethers or esters, acids, bases, solvates, hydrates or prodrugs. The concentrations of the compounds in the compositions are effective for delivery of an amount, upon administration, that treats, prevents, or ameliorates one or more of the symptoms of at least one psychotic condition, such as schizophrenia.

In some embodiments, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions may also be suitable as pharmaceutically acceptable carriers. These may be prepared according to methods known to those skilled in the art. For example, in an embodiment, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline (PBS) lacking divalent cations is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate at least one symptom of a psychotic condition, such as schizophrenia.

In some embodiments, a therapeutically effective dosage should produce a serum concentration of active ingredient of about 0.1 ng/ml to about 100 μg/ml. The pharmaceutical compositions, in certain embodiments, should provide a dosage of about 0.001 mg to about 2000 mg of compound per kilogram of body weight per day. Pharmaceutical dosage unit forms are prepared to provide from about 1 mg to about 1000 mg of active ingredient in an embodiment. In another embodiment, the dosage unit form provides from about 10 mg to about 500 mg of the active ingredient. A combination of active ingredients may be packaged per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding parent or underivatized compound.

Effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing a psychotic condition, such as schizophrenia. The concentration of active compound in the composition will depend on absorption, inactivation, excretion rates of the active compound, the dosage schedule, amount administered, the particular formulation used, and other factors known to those of skill in the art.

Pharmaceutical compositions are intended to be administered by a suitable route, including orally, parenterally, rectally, topically and locally. For oral administration, capsules and tablets can be used. The compositions are in liquid, semi-liquid or solid form and are formulated in a manner suitable for each route of administration. In some embodiments, modes of administration include parenteral and oral modes of administration. In some embodiments, oral administration is contemplated.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, bags, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, or emulsion. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple-dose form is a multiple of unit-doses which are not segregated in packaging.

Sustained-release preparations can also be prepared. Suitable examples of sustained-release preparations include semi-permeable matrices of solid hydrophobic polymers containing the compound provided herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3 hydroxybutyric acid. Polymers such as ethylene-vinyl acetate and lactic acid-glycolic acid enable release of molecules for over 100 days, although other polymers release molecules over a shorter time period.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, and polylactic acid. Methods for preparation of these compositions are known to those skilled in the art. The contemplated compositions may contain 0.001-100% active ingredient. In some embodiments, the composition may be 0.1-85% active ingredient. In other embodiments, the composition may be 75-95% active ingredient.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds and pharmaceutically acceptable derivatives thereof provided herein, may also be advantageously administered for therapeutic or prophylactic purposes together with another pharmacological agent known in the general art to be of value in treating one or more of psychotic conditions. It is to be understood that such combination therapy constitutes a further aspect of the compositions and methods of treatment provided herein.

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant, a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof, and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, can be encapsulated in a gelatin capsule. Such solutions, and the preparation and encapsulation thereof are known in the art. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene, glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, dialkyl acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art. Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In certain embodiments, a therapeutically effective dosage is formulated to contain a concentration of at least about 0.1% w/w up to about 90% w/w or more, or more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, 5-35 mg or about 9-30 mg of lyophilized powder, is added per mL of sterile water or other suitable carrier. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds and pharmaceutically acceptable derivatives thereof may be formulated as aerosols for topical application as is known in the art. Administration may be in aerosol form. Formulations for administration in aerosol form may be in the form of an aerosol or solution for a nebulizer, or as a microfine powder for inhalation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01-10% isotonic solutions, pH about 5-7, with appropriate salts.

Other routes of administration, such as topical application, transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. In certain embodiments, the weight of a rectal suppository is about 2 to 3 gm.

Tablets and capsules for rectal administration are manufactured using the same pharmaceutically acceptable substance and by the same methods as for formulations for oral administration.

Active ingredients such as the compounds provided herein can be administered by controlled release means or by delivery devices that are well known to those of ordinary skill in the art. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus, the compositions provided encompass single unit dosage forms suitable for oral administration such as, but not limited to, tablets, capsules, gelcaps, and caplets that are adapted for controlled release.

All controlled release pharmaceutical products have a common goal of improving drug therapy over that achieved by their non-controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the drug may be administered using intravenous infusion, an implantable osmotic pump, a transdermal patch, liposomes, or other modes of administration. In another embodiment, polymeric materials can be used. In yet another embodiment, a controlled release system can be placed in a subject at an appropriate site determined by a practitioner of skill, i.e., thus requiring only a fraction of the systemic dose. The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylenevinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

The compounds provided herein, or pharmaceutically acceptable derivatives thereof may also be formulated to be targeted to a particular tissue, receptor, or other area of the body of the subject to be treated. Many such targeting methods are well known to those of skill in the art. All such targeting methods are contemplated herein for use in the instant compositions.

The compounds or pharmaceutically acceptable derivatives can be packaged as articles of manufacture containing packaging material, a compound or pharmaceutically acceptable derivative thereof provided herein, which is used for treatment, prevention or amelioration of one or more symptoms associated with an antipsychotic condition, such as schizophrenia.

The articles of manufacture provided herein contain packaging materials. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated.

### EXPERIMENTAL EXAMPLES

The following examples are included to demonstrate particular aspects of the present disclosure. It should be appreciated by those of ordinary skill in the art that the methods described in the examples that follow merely represent exemplary embodiments of the disclosure. Those of skill in the art should, in light of the present disclosure, appreciate that many changes can be made in the specific embodiments described and still obtain a like or similar result without departing from the spirit and scope of the present disclosure.

### Example 1A

### D₂, 5-HT_{2A} and 5-HT_{1A} Receptor Binding Model Results

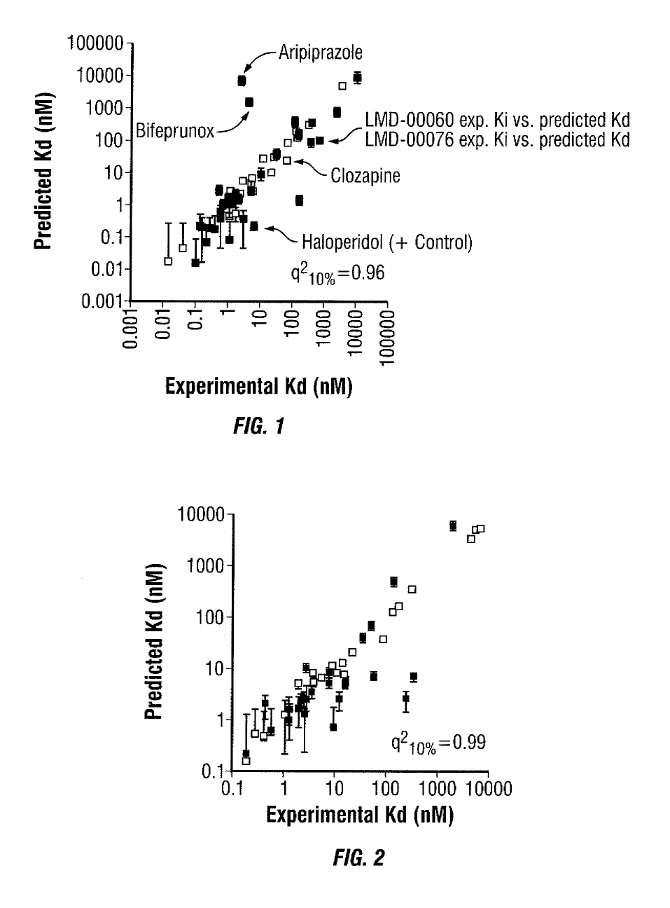

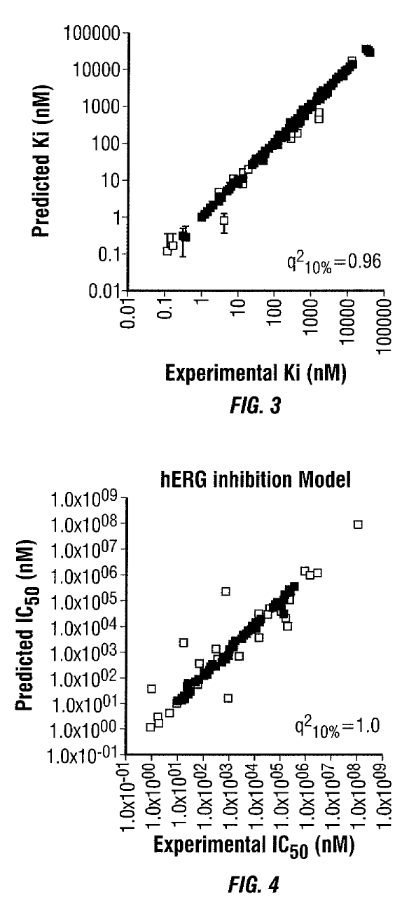

For a library of >250 compounds disclosed herein which were identified by spectral modeling to have promising 5-HT_{2A} and D₂ receptor activity, predicted K_{d} and Kᵢ values were determined for D₂, 5-HT_{2A} and 5-HT_{1A}. FIG. 1 presents a plot of predicted versus experimental K_{d} for the D₂ receptor. FIG. 2 presents a plot of predicted versus experimental K_{d} for the 5-HT_{2A} receptor. FIG. 3 presents a plot of predicted versus experimental K_{d} for the 5-HT_{1A} receptor. Both validation and cross-validation data is shown.

### Example 1B

### hERG Inhibition, Antipsychotic-Associated Weight Gain and Agranulocytosis Risk Model Results

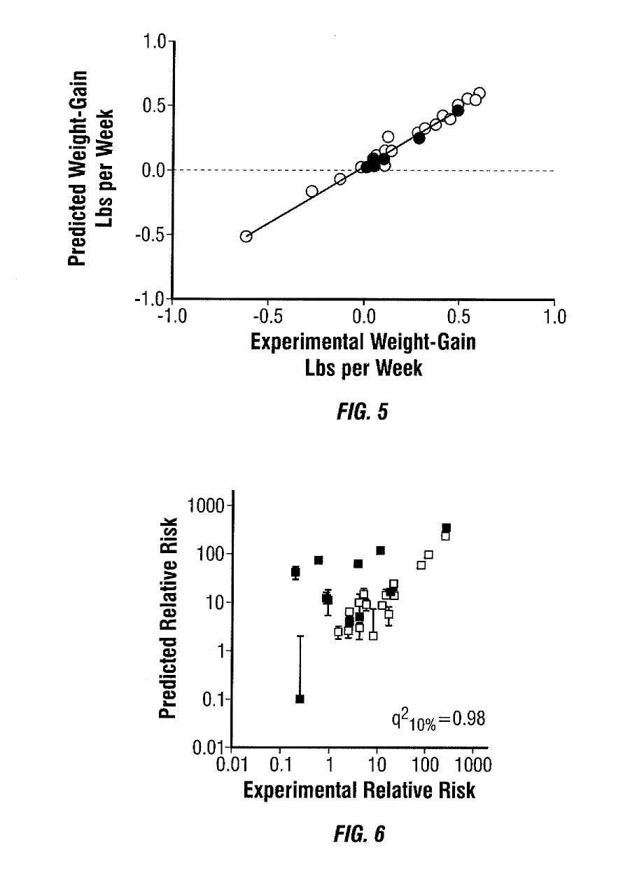

Predicted versus experimental risk factors for hERG inhibition, antipsychotic-associated weight gain and agranulocytosis risk were determined for the library of compounds disclosed herein. FIG. 4 presents a plot of predicted versus experimental IC₅₀ for hERG inhibition. FIG. 5 presents a plot of predicted versus experimental antipsychotic-associated weight gain. FIG. 6 presents a plot of predicted versus experimental agranulocytosis relative risk. Both validation and cross-validation data is shown.

### Example 1C

### Selection of Compounds from Spectral Modeling Studies

Based on predicted receptor affinities and toxicities of the compounds identified by spectral modeling studies, three compounds were identified for synthesis and further testing. These compounds are identified below.

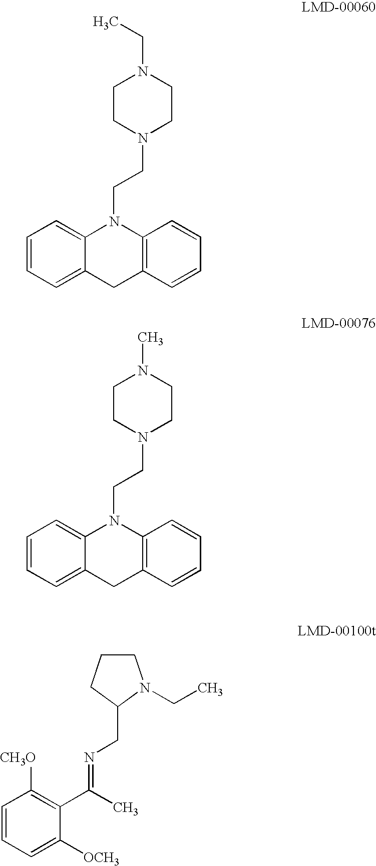

### Example 1D

### In Vitro of LMD-00060 and LMD-00076

In vitro testing data of LMD-00060 and LMD-00076 across a broad range of receptor targets is summarized in Table 1 below.

TABLE 1
In Vitro Testing Data for LMD-00060 and LMD-00076

| | LMD-00060 | LMD-00076 |
|---|---|---|
| Receptor | (nM) | (nM) |
| 5-HT₁ₐ | — | yes |
| 5-HT₂ₐ | 349 | 249 |
| 5-HT_{2c} | 2741 | 968 |
| 5-HT₆ | 1522 | 652 |
| 5-HT₇ | 1057 | 1136 |
| Alpha 1A | yes | yes |
| Alpha 1B | yes | yes |
| Alpha 1D | yes | yes |
| Alpha 2A | yes | yes |
| Alpha 2B | yes | yes |
| Alpha 2C | yes | yes |
| D₂ | 632 | 692 |
| H₁ | 255 | 185 |
| M₁ | 1358 | 707 |
| M₂ | >10,000 | 3334 |
| M₃ | 2493 | 702 |
| M₄ | 1913 | 1101 |
| M₅ | 613 | 419 |

The in vitro screen results desirably demonstrated a greater affinity for 5-HT_{2A} receptors than for D₂ receptors. Furthermore, in cross-screening against other receptors, the affinities were generally greater for 5-HT_{2A} than for the other receptors.

### Example 2

### Synthesis of 5-(2-(4-ethylpiperazin-1-yl)ethyl)-5,10-dihydrophenazine and 5-(2-(4-methylpiperazin-1-yl)ethyl)-5,10-dihydrophenazine (LMD-00060 and LMD-00076)

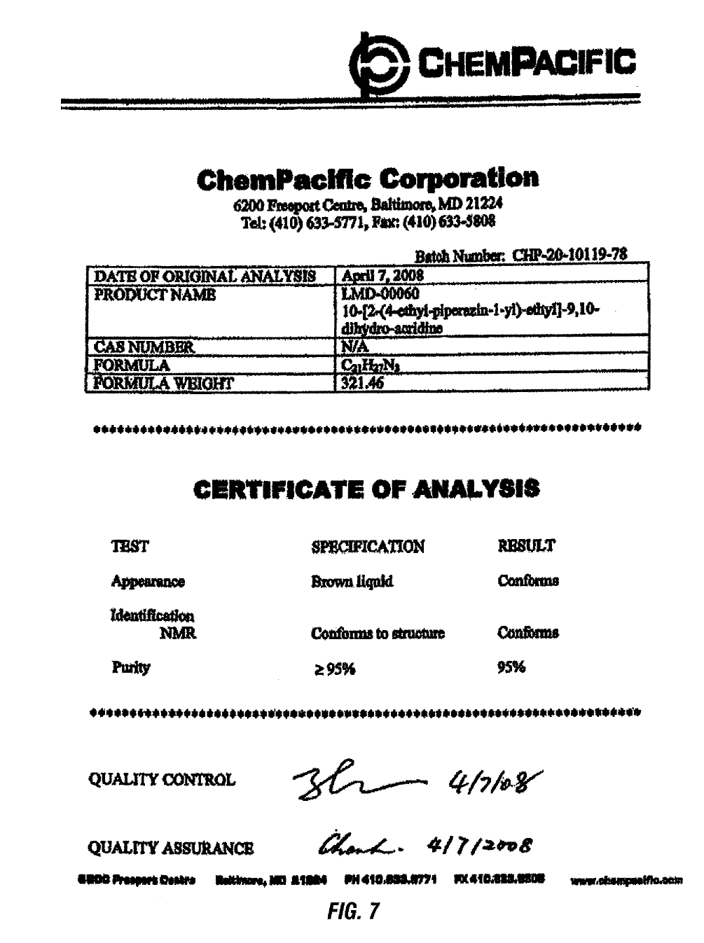

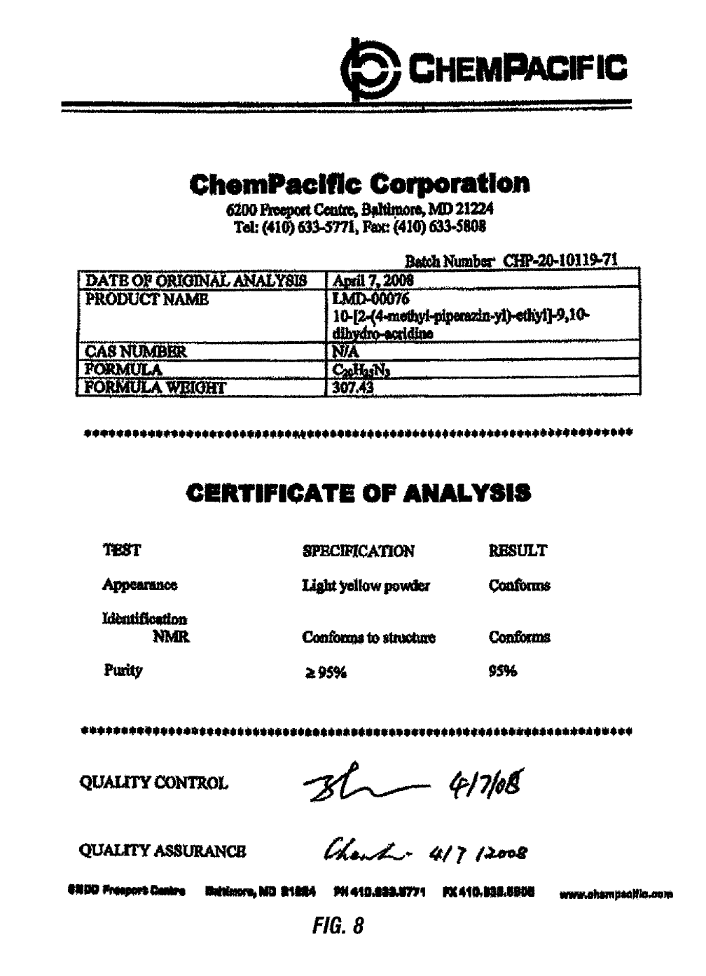

Compounds 5-(2-(4-ethylpiperazin-1-yl)ethyl)-5,10-dihydrophenazine (LMD-00060) and 5-(2-(4-methylpiperazin-1-yl)ethyl)-5,10-dihydrophenazine (LMD-00076) were synthesized commercially by ChemPacific Corporation (Baltimore, Md.). Scheme 1 shows the general synthetic protocol used to synthesize the compounds. FIGS. 7 and 8 present Certificates of Analysis for LMD-00060 and LMD-00076, respectively. The Certificates of Analysis provide analytical data attesting to the identity and purity of the compounds.

[symbol]

### Example 3

### Synthesis of (E)-N-(1-(2,6-dimethoxyphenyl)ethylidene)-1-(1-ethylpyrrolidin-2-yl)methanamine (LMD-00100t)

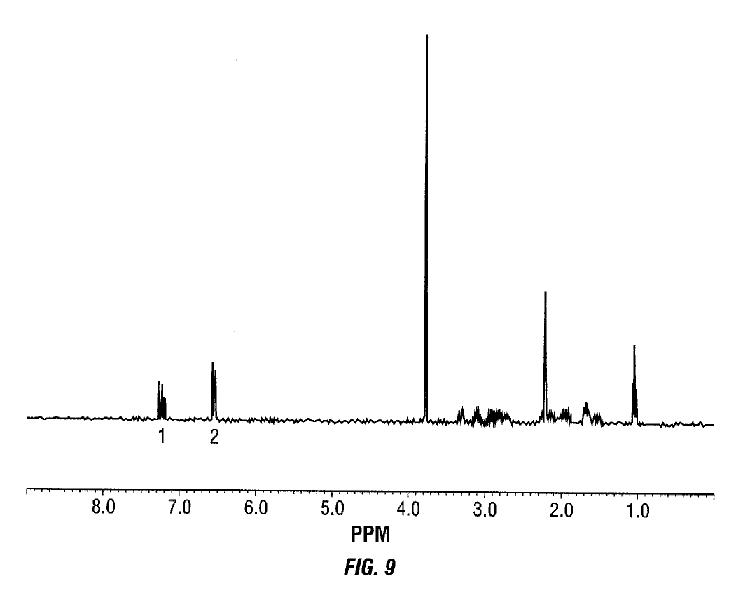

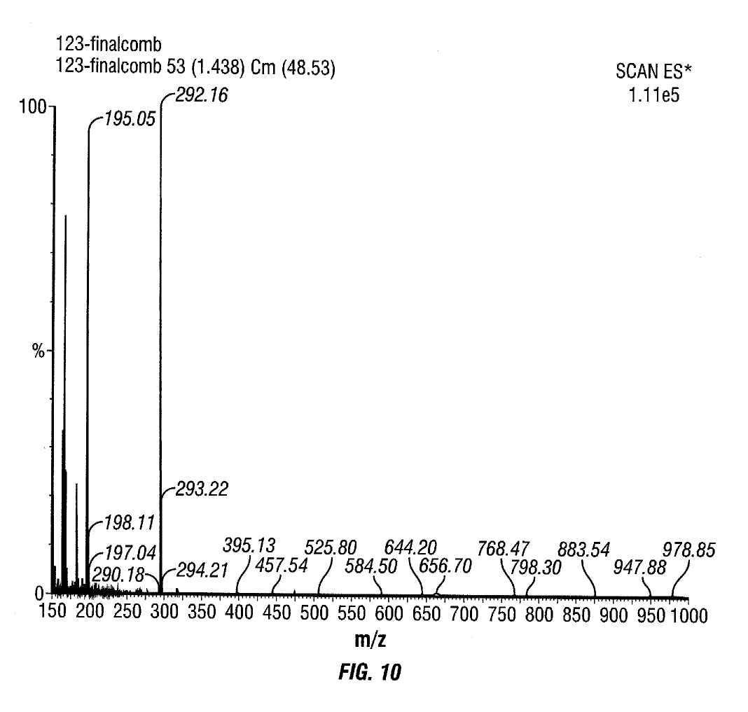

Compound (E)-N-(1-(2,6-dimethoxyphenyl)ethylidene)-1-(1-ethylpyrrolidin-2-yl)methanamine (LMD-00100t) was synthesized by MedChem Partners (Medford, Mass.). The synthetic protocol follows. In a round bottom flask under nitrogen, 6.5 g of 2,6-dimethoxyacetophenone (36.1 mmol) and 6.5 g of 2(aminomethyl)-1-ethyl pyrrolidine (50.8 mmol) were dissolved in 100 mL of anhydrous methanol, and 12 g of magnesium sulfate (99.7 mmol) was added. The mixture was heated to reflux for 18 hours. After cooling to room temperature, the solids were removed by filtration and the filtrate was concentrated in vacuo. The resulting crude material was purified by flash chromatography (SiO₂, CH₂Cl₂/MeOH gradient) to give the desired compound (4.4 g, 42%). FIG. 9 shows the ¹H NMR of LMD-00100t. FIG. 10 shows the LC-MS of LMD-00100t. The ¹H NMR is consistent with the assigned structure. The LC-MS shows an M+2H molecular ion peak of 292.16 consistent with the assigned structure. The general synthetic scheme for LMD-00100t is shown in Scheme 2.

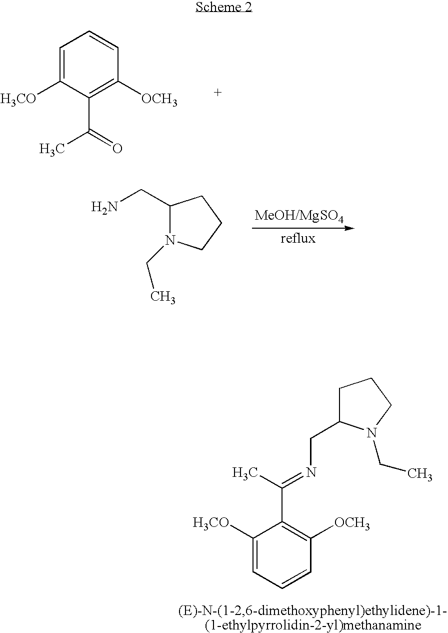

### Example 4

### In Vivo Microdialysis Studies of LMD-00076 and LMD-00100t

In vivo microdialysis studies were conducted according to established methods. In summary, two cannulae with microdialysis probes were implanted via stereotaxic surgery (one each into two separate brain regions), into the medial prefrontal cortex and nucleus accumbens of male Sprague-Dawley rats weighing from 250 g to 300 g. After recovery from surgery, a solution was circulated through the probes on test days collecting extracellular synaptic fluid at 30-minute intervals and delivering it to an HPLC for analysis of extracellular dopamine concentrations in real time following antipsychotic compound dosing. Simultaneously, an aliquot was taken and frozen for later mass spectrometric determination of acetylcholine concentrations.

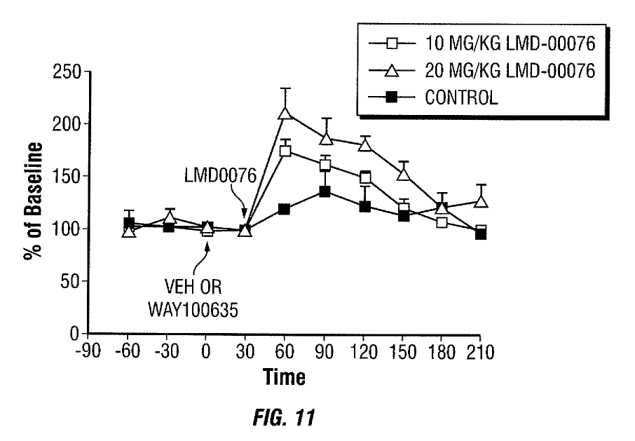

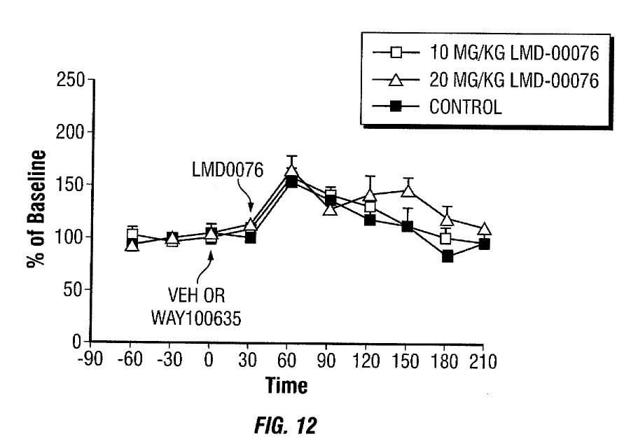

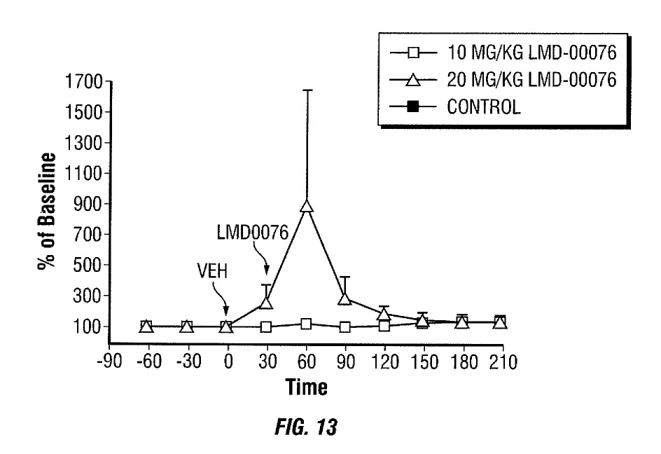

FIG. 11 shows a plot of pre-frontal cortex dopamine release as a function of time following administration of LMD-00076. The response was dose-dependent and consistent with atypical antipsychotic activity. The curve labeled control showed that dopamine efflux was blocked when 5-HT_{1A} silent agonist WAY 100635 was co-dosed with LMD-00076. FIG. 12 shows a plot of nucleus accumbens dopamine release as a function of time following administration of LMD-00076. The increased nucleus accumbens dopamine release is also consistent with atypical antipsychotic activity. FIG. 13 shows a plot of pre-frontal cortex acetylcholine release as a function of time following administration of LMD-00076. The increased acetylcholine release is also consistent with atypical antipsychotic activity.

LMD-00076 produced a modest increase in nucleus accumben dopamine release that exhibited a ceiling effect where increasing dose did not produce additional transmitter release (see FIG. 12). LMD-00076 also produced a significant increase in cortical acetylcholine (ACh) efflux, similar to that of direct acting 5-HT_{2A}/D₂ antagonists (see FIG. 13). Taken collectively, these results indicate that LMD-00076 produced neurotransmitter release patterns similar to those of clozapine in a rigorous animal model of atypical antipsychotic activity. Furthermore, such Ach efflux is associated with positive effects on cognitive function.

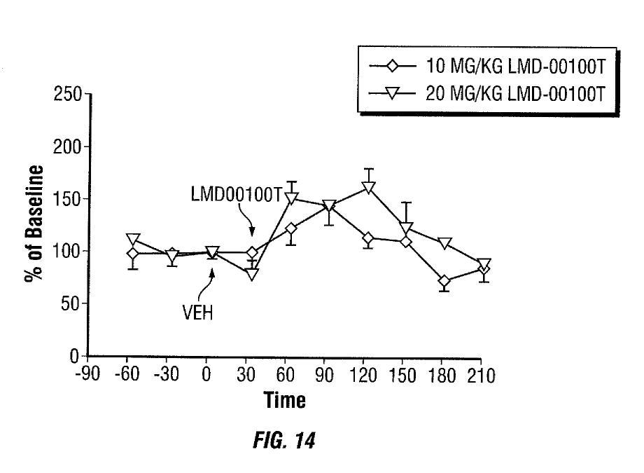

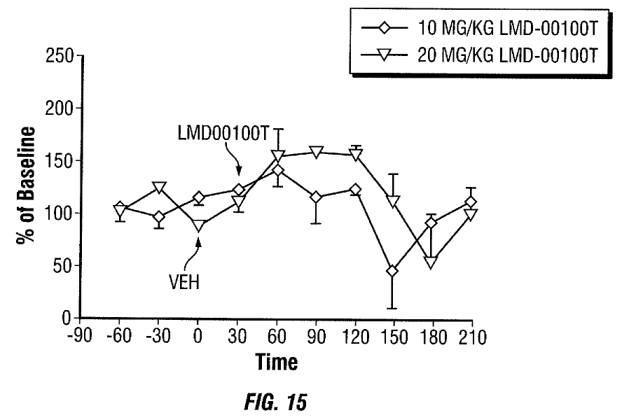

FIG. 14 shows a plot of pre-frontal cortex dopamine release as a function of time following administration of LMD-00100t. FIG. 15 shows a plot of nucleus accumbens dopamine release as a function of time following administration of LMD-00100t.

LMD-00100t produced a significant increase in cortical dopamine release (see FIG. 14), but at the maximum dose tested, the compound did not increase cortical ACh efflux (data not shown). This pattern resembles that of the antipsychotic aripiprazole. LMD-00100t was predicted by spectral modeling to be a very weak D₂ antagonist (K_{d} value>1200 nM). Accordingly LMD-00100t was proportionally less potent in vivo. The in vivo microdialysis profile of LMD-00100t resembled that of aripiprazole which is a partial D₂ agonist. However, the in vivo results of LMD-00100t relative to LMD-00076 suggests that it is, in fact, a weak antagonist.

From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure, which is defined in the following claims.

What is claimed is the following:

1. A compound having a structure of: 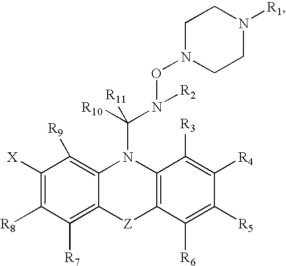
tautomers and stereoisomers thereof, and pharmaceutical derivatives thereof,
wherein X is selected from the group consisting of H, F, Cl, and Br;
wherein Z is selected from the group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂;
wherein R₁₂ is selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₃ is selected from the group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₄ and R₁₅ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio;
wherein R₁ and R₂ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl;
wherein R₃-R₉ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio; and
wherein R₁₀ and R₁₁ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, and heteroarylalkyl.

2. The compound of claim 1, wherein said compound has a structure selected from the group consisting of 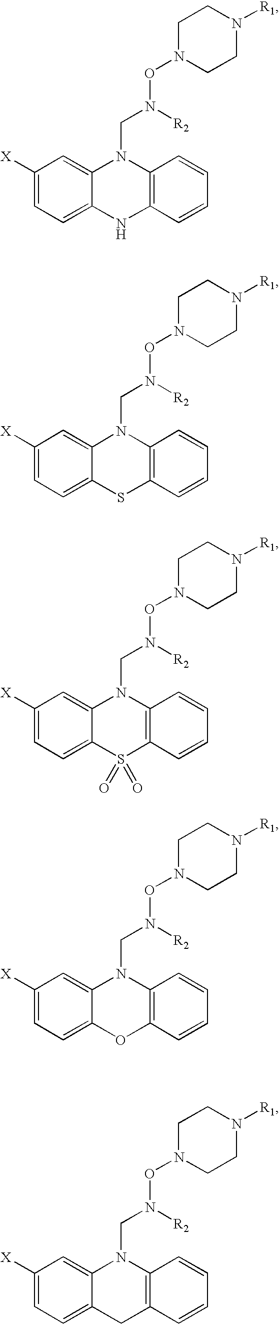 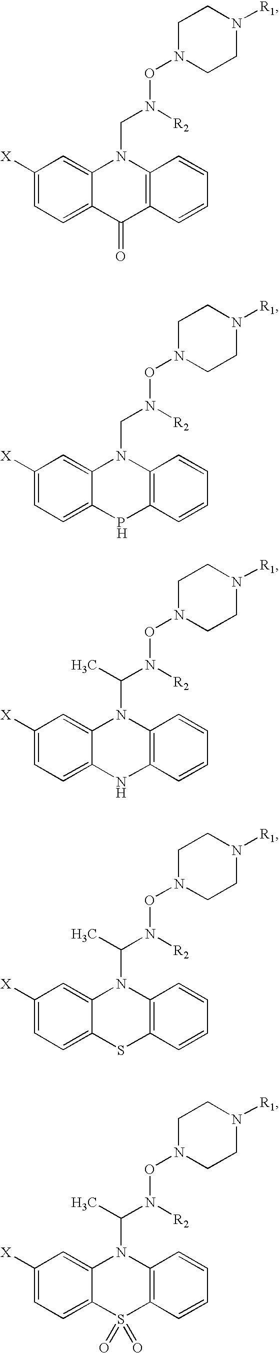 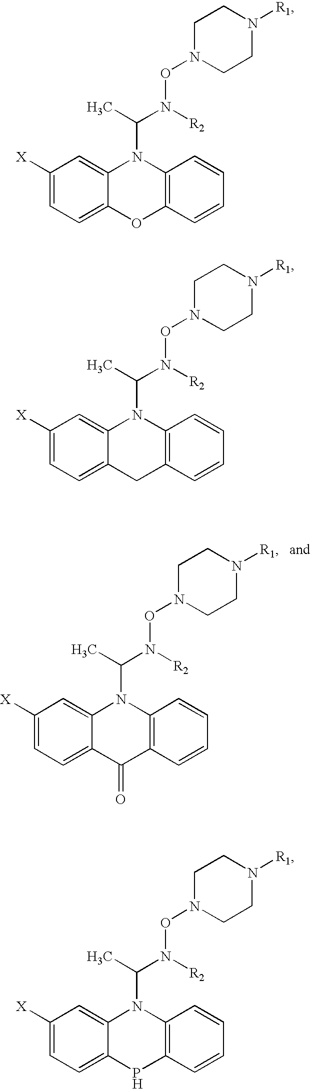
tautomers and stereoisomers thereof, and pharmaceutical derivatives thereof.

3. The compound of claim 2, wherein said compound has a structure selected from the group consisting of 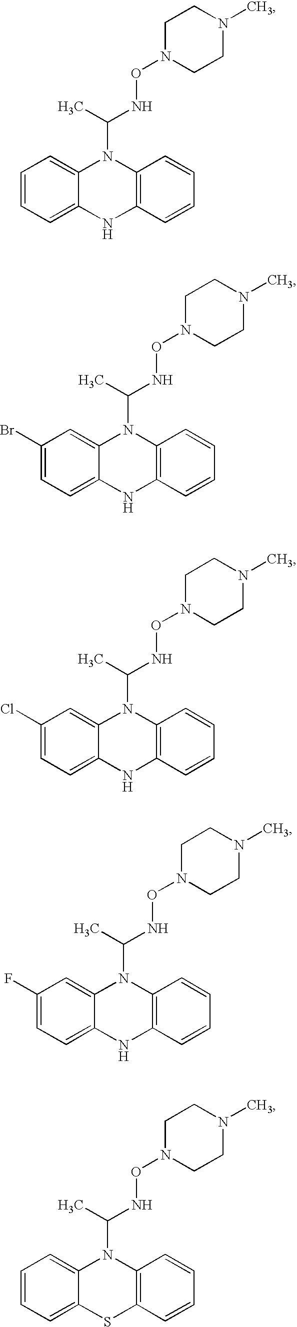 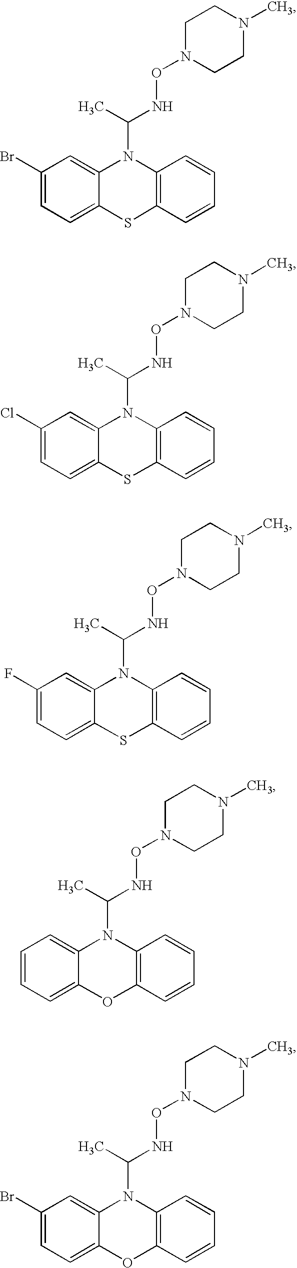 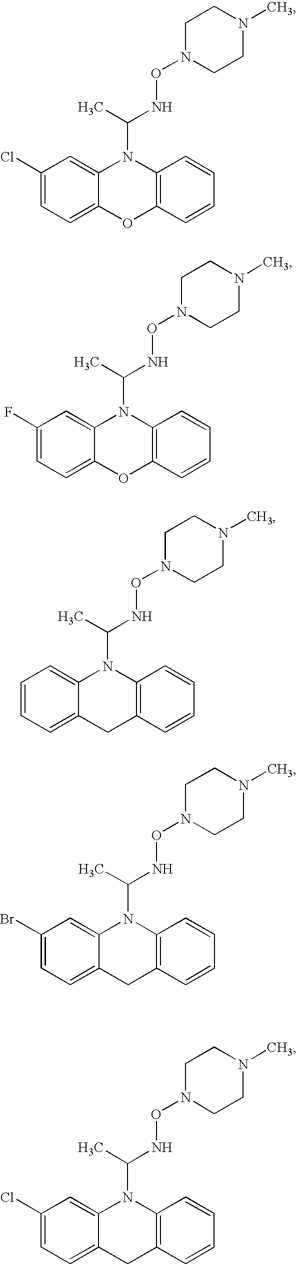 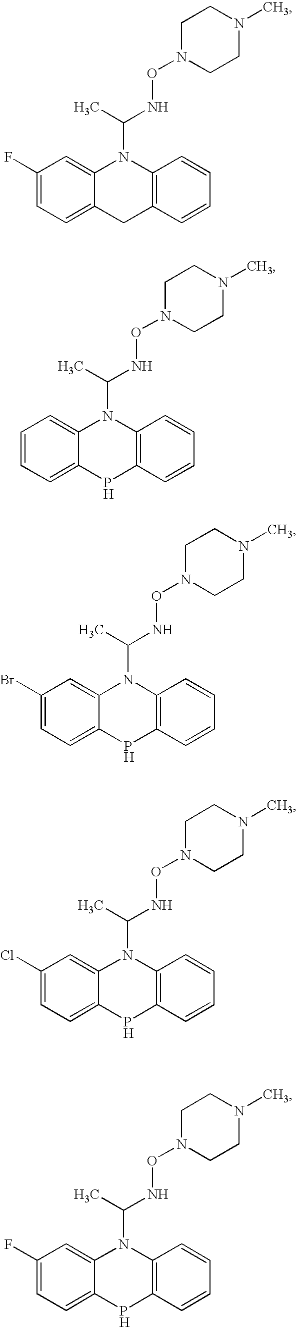 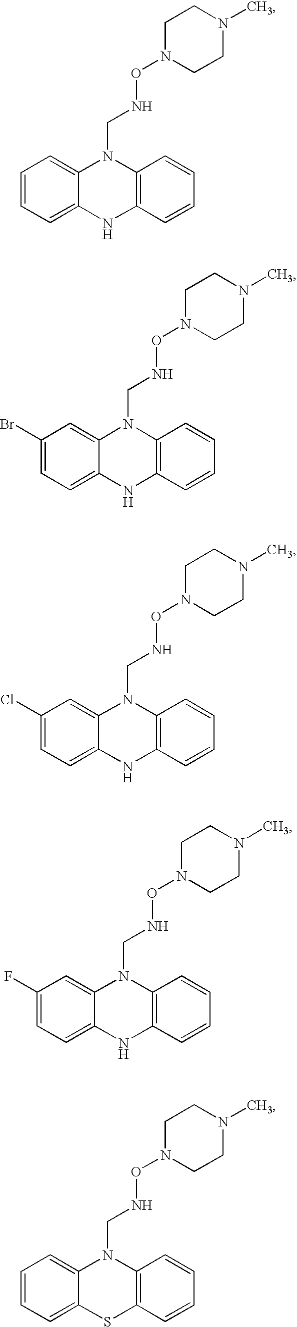 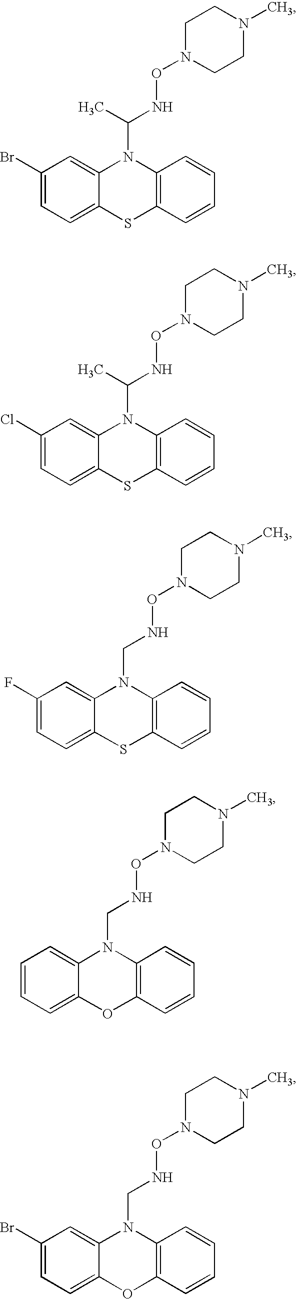 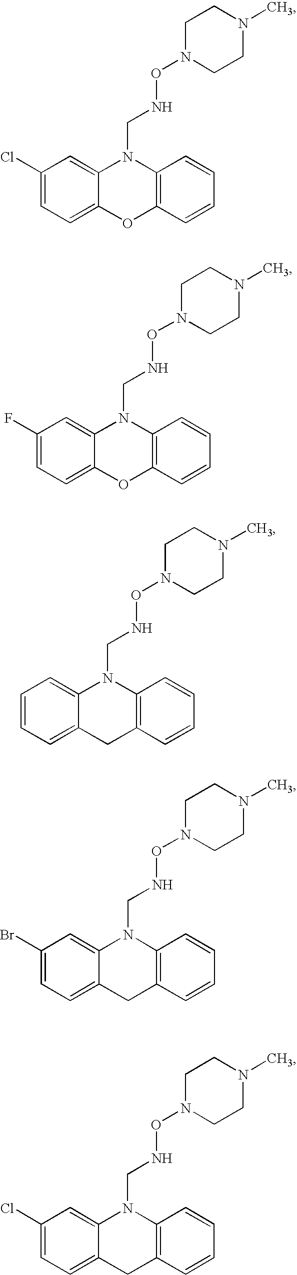 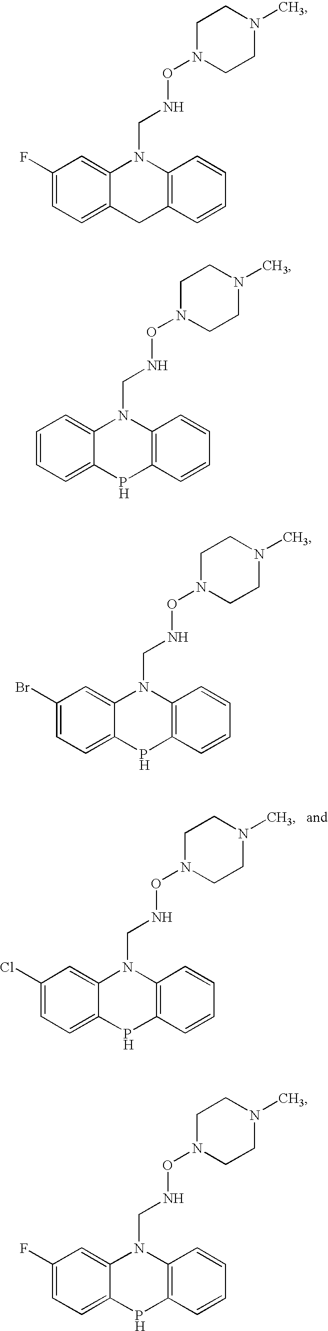
tautomers and stereoisomers thereof, and pharmaceutical derivatives thereof.

4. A compound having a structure of: 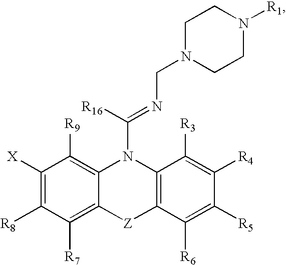
tautomers and geometric isomers thereof, and pharmaceutical derivatives thereof,
wherein X is selected from the group consisting of H, F, Cl, and Br;
wherein Z is selected from the group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂;
wherein R₁₂ is selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₃ is selected from the group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₄ and R₁₅ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio;
wherein R₁ is selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl;
wherein R₃-R₉ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N-diaryl-N-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio; and
wherein R₁₆ is selected from the group consisting of alkyl, alkenyl, alkynyl, heteroalkyl, aralkyl, cycloalkyl, cycloalkylalkyl, heterocyclyl, heterocyclylalkyl, and heteroarylalkyl.

5. The compound of claim 4, wherein said compound has a structure selected from the group consisting of 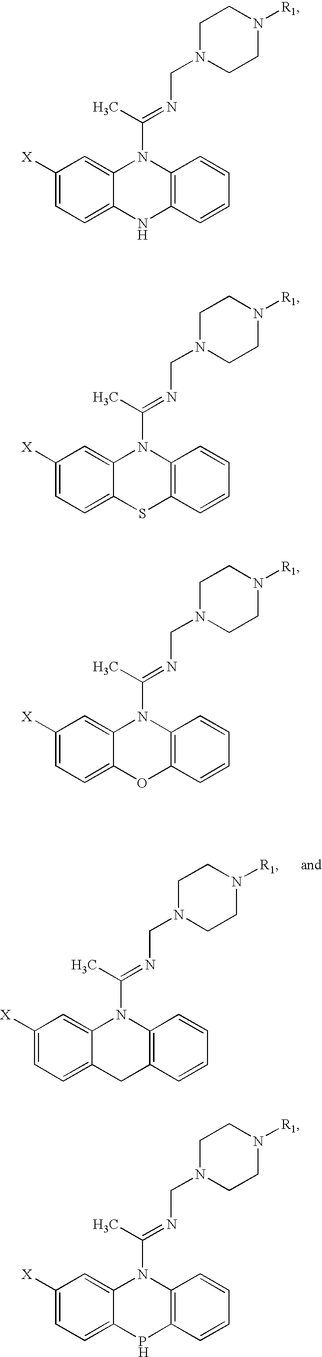
tautomers and geometric isomers thereof, and pharmaceutical derivatives thereof.

6. The compound of claim 5, wherein said compound has a structure selected from the group consisting of 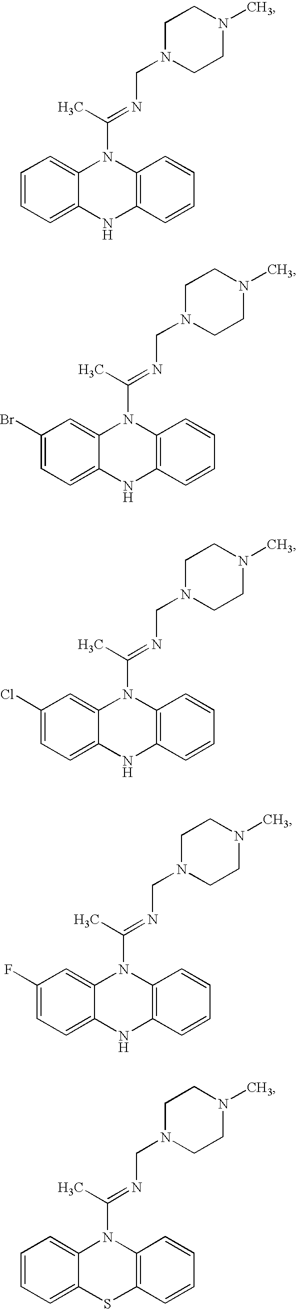 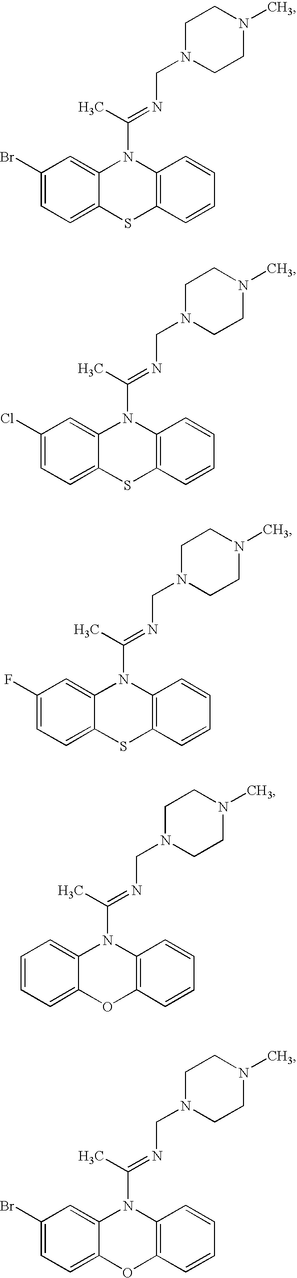 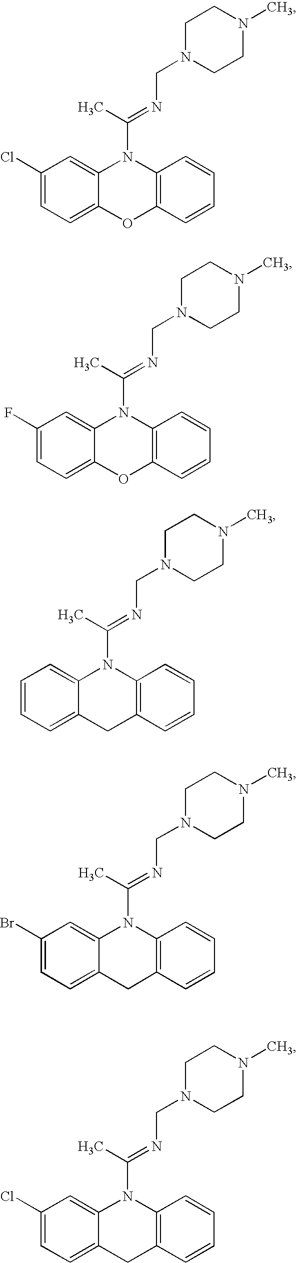 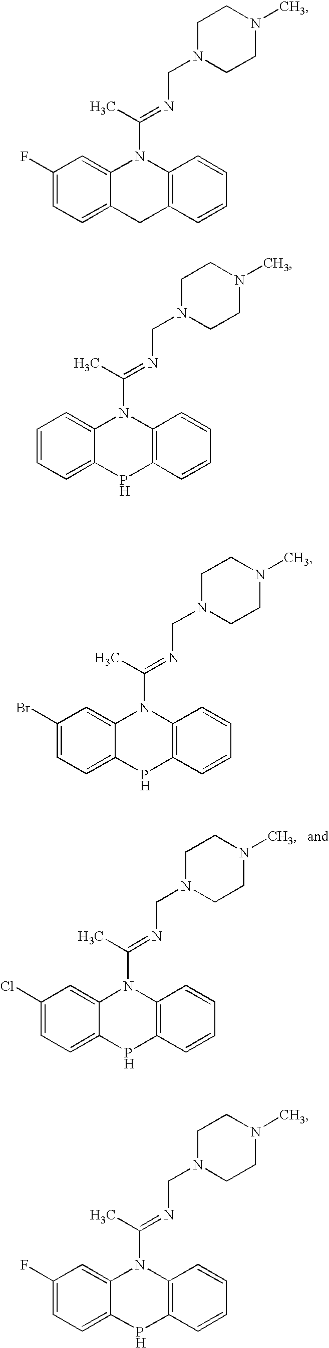
tautomers and geometric isomers thereof, and pharmaceutical derivatives thereof.

7. A compound having a structure of: 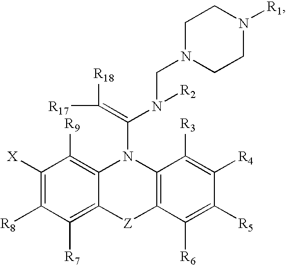
tautomers and geometric isomers thereof, and pharmaceutical derivatives thereof,
wherein X is selected from the group consisting of H, F, Cl, and Br;
wherein Z is selected from the group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂;
wherein R₁₂ is selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₃ is selected from the group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₄ and R₁₅ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio;
wherein R₁ and R₂ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl;
wherein R₃-R₉ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio; and
wherein R₁₇ and R₁₈ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

8. The compound of claim 7, wherein said compound has a structure selected from the group consisting of 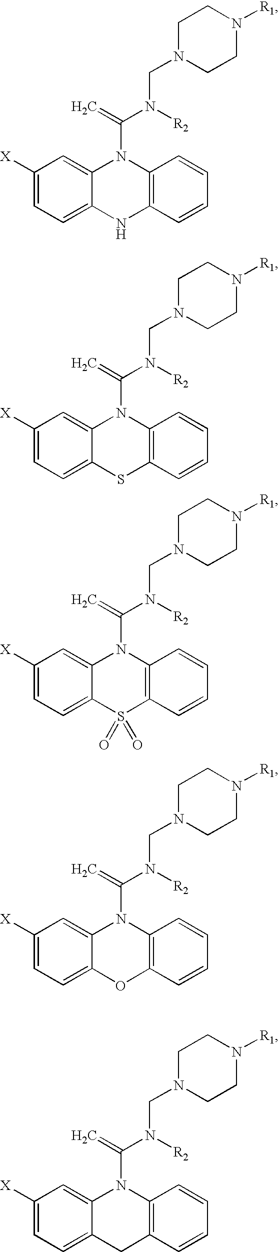 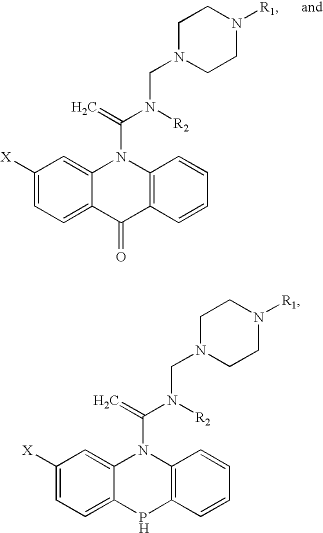
tautomers thereof, and pharmaceutical derivatives thereof.

9. The compound of claim 8, wherein said compound has a structure selected from the group consisting of 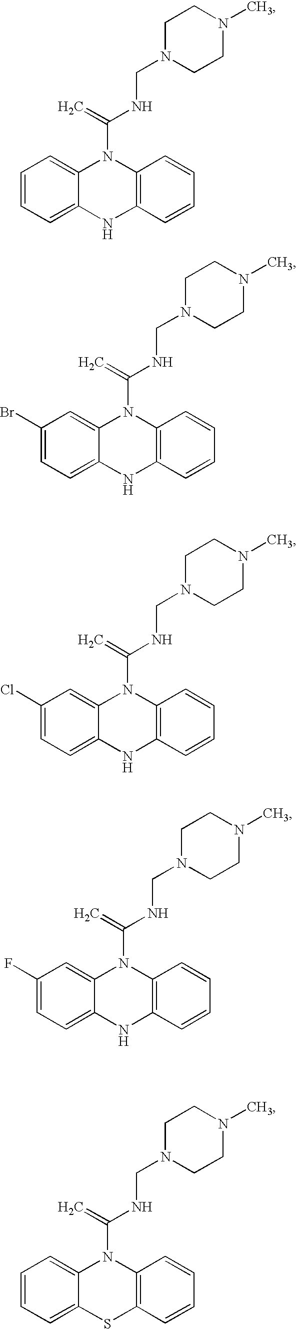 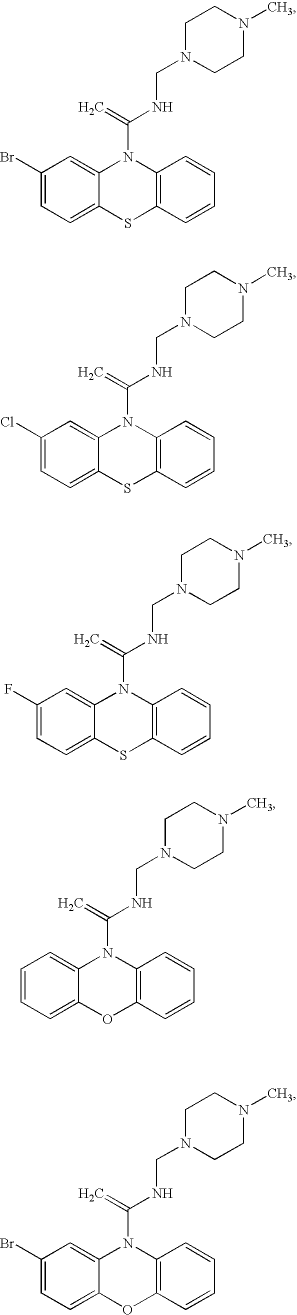 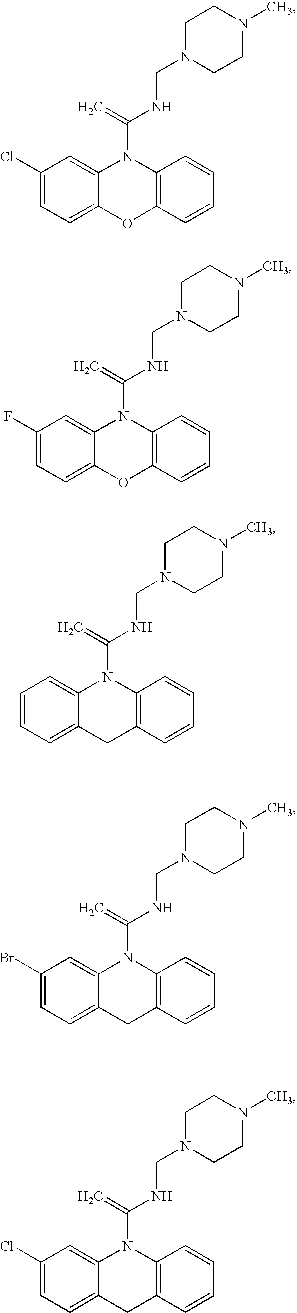 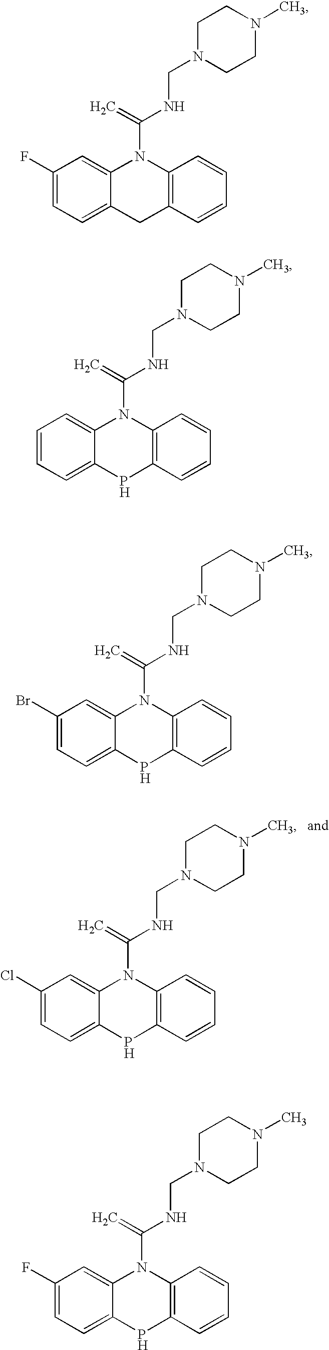
tautomers thereof, and pharmaceutical derivatives thereof.

10. The compound of claim 8, where said compound has a structure selected from the group consisting of 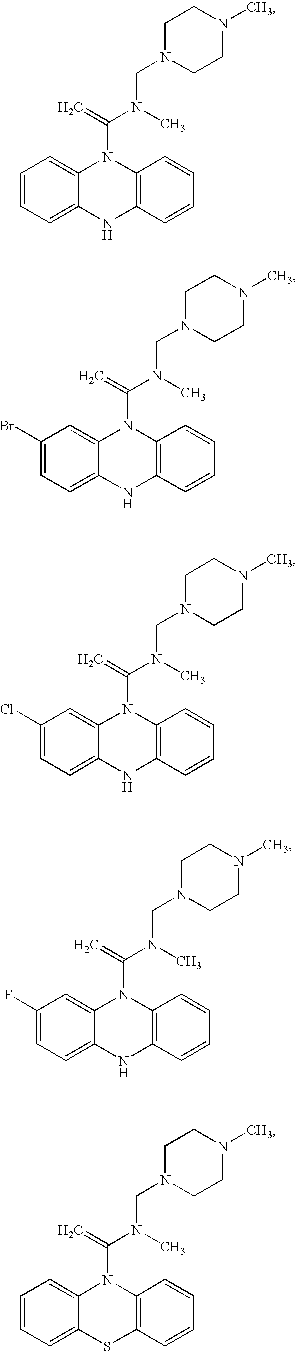 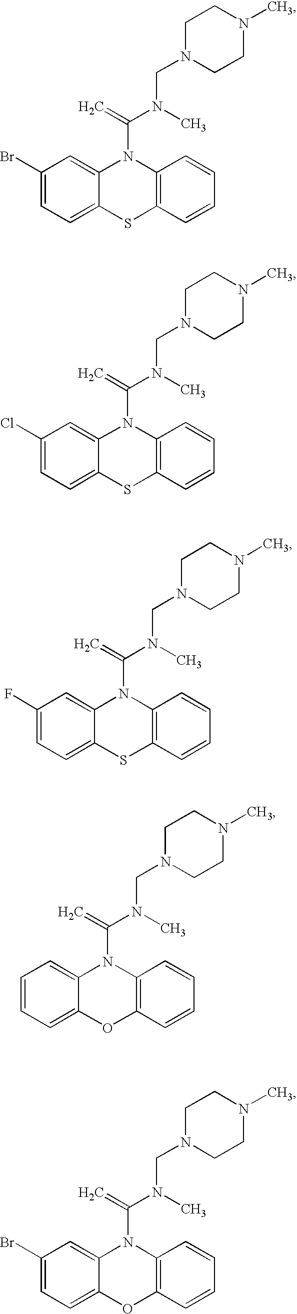 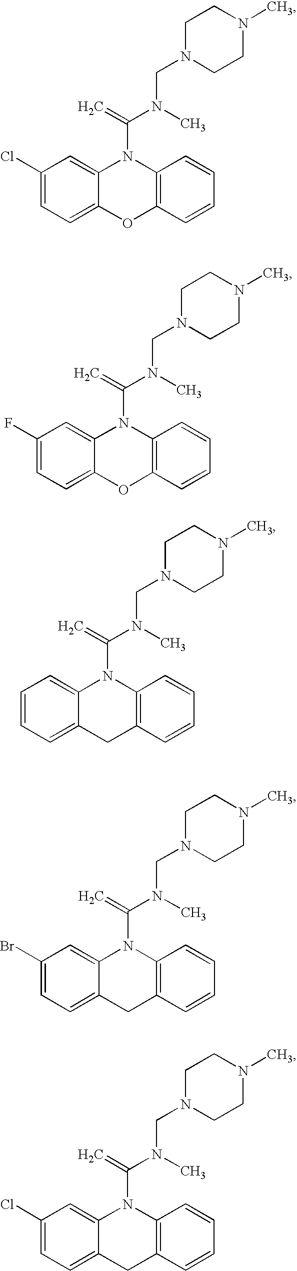 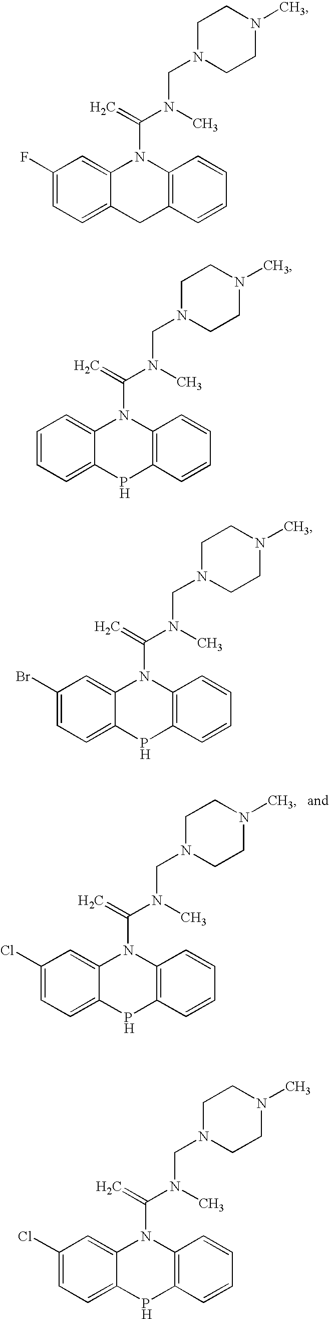
and pharmaceutical derivatives thereof.

11. A compound having a structure of: 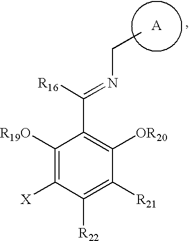
tautomers, stereoisomers, and geometric isomers thereof, and pharmaceutical derivatives thereof,
wherein A is a ring selected from the group consisting of a heterocyclic ring and a heteroaromatic ring,
wherein 3 to 9 carbon atoms and 1 to 4 nitrogen atoms comprise said ring;
wherein X is selected from the group consisting of H, F, Cl, and Br;
wherein R₁₆ is selected from the group consisting of alkyl, alkenyl, alkynyl, heteroalkyl, aralkyl, cycloalkyl, cycloalkylalkyl, heterocyclyl, heterocyclylalkyl, and heteroarylalkyl;
wherein R₁₉ and R₂₀ are independently selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl; and
wherein R₂₁ and R₂₂ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio.

12. The compound of claim 11, wherein said compound has a structure selected from the group consisting of 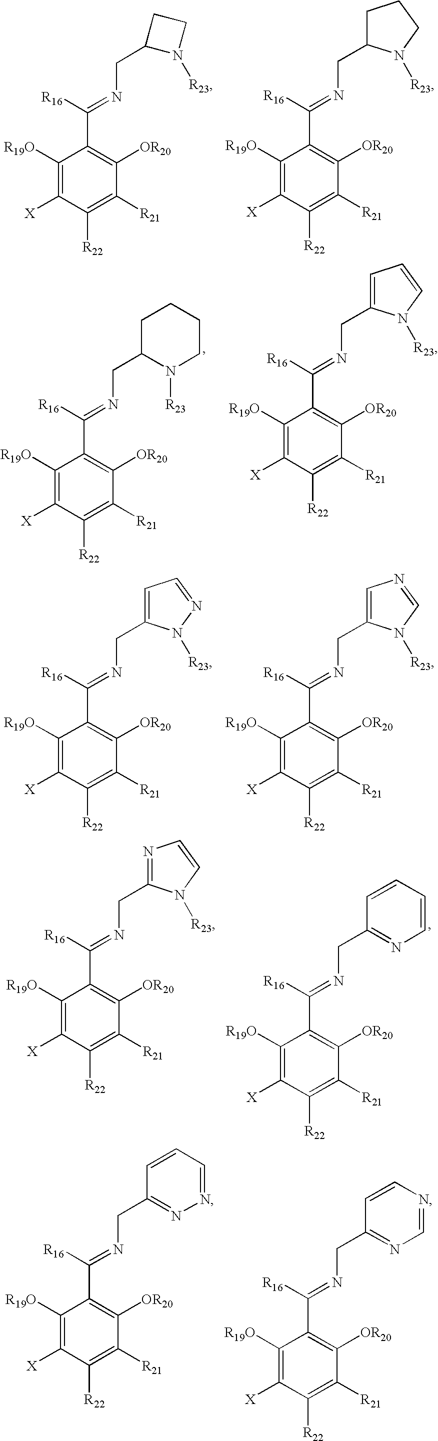 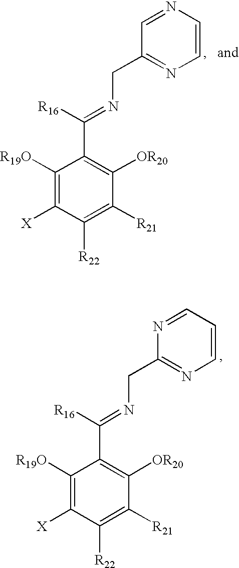
tautomers, stereoisomers, and geometric isomers thereof and pharmaceutical derivatives thereof,
wherein R₂₃ selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

13. The compound of claim 12, wherein said compound has a structure selected from the group consisting of 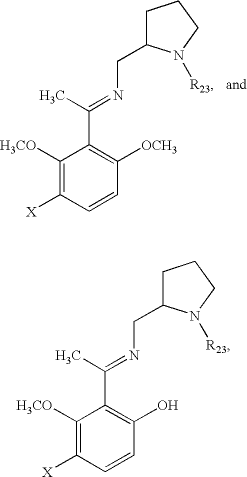
tautomers, stereoisomers, and geometric isomers thereof, and pharmaceutical derivatives thereof.

14. The compound of claim 13, wherein said compound has a structure selected from the group consisting of 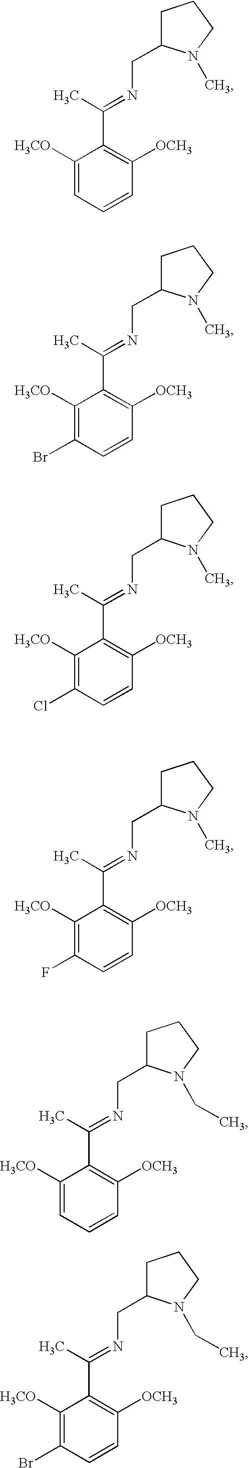 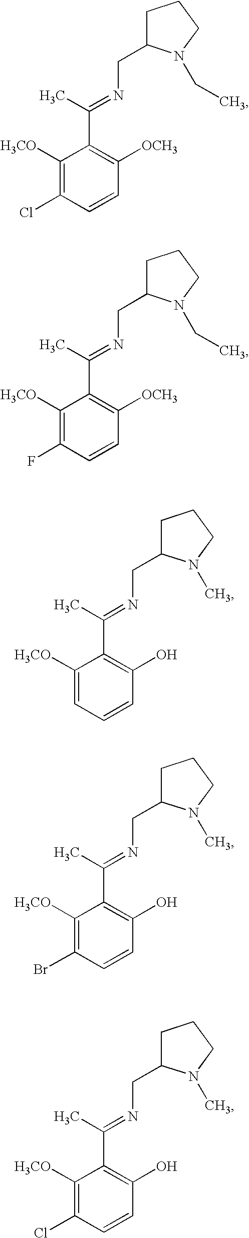 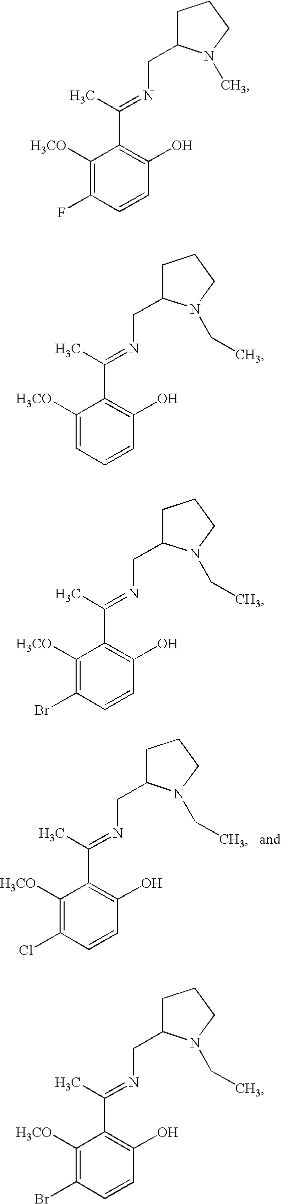
tautomers, stereoisomers, and geometric isomers thereof, and pharmaceutical derivatives thereof.

15. A compound having a structure of: 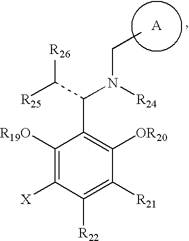
tautomers, stereoisomers, and geometric isomers thereof, and pharmaceutical derivatives thereof,
wherein A is a ring selected from the group consisting of a heterocyclic ring and a heteroaromatic ring,
wherein 3 to 9 carbon atoms and 1 to 4 nitrogen atoms comprise said ring;
wherein the dashed bond comprises a bond selected from the group consisting of a single bond and a double bond;
wherein X is selected from the group consisting of H, F, Cl, and Br;
wherein R₁₉ and R₂₀ are independently selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, and heteroarylalkylcarbonyl;
wherein R₂₁ and R₂₂ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycaxbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio;
wherein R₂₄ is selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl; and
wherein R₂₅ and R₂₆ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

16. The compound of claim 15, wherein said compound has a structure selected from the group consisting of 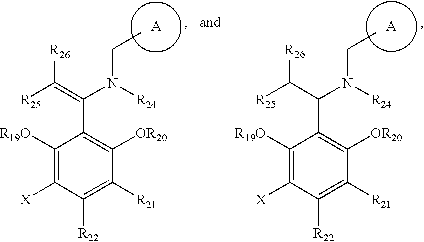
tautomers, stereoisomers, and geometric isomers thereof, and pharmaceutical derivatives thereof.

17. The compound of claim 16, wherein said compound has a structure selected from the group consisting of 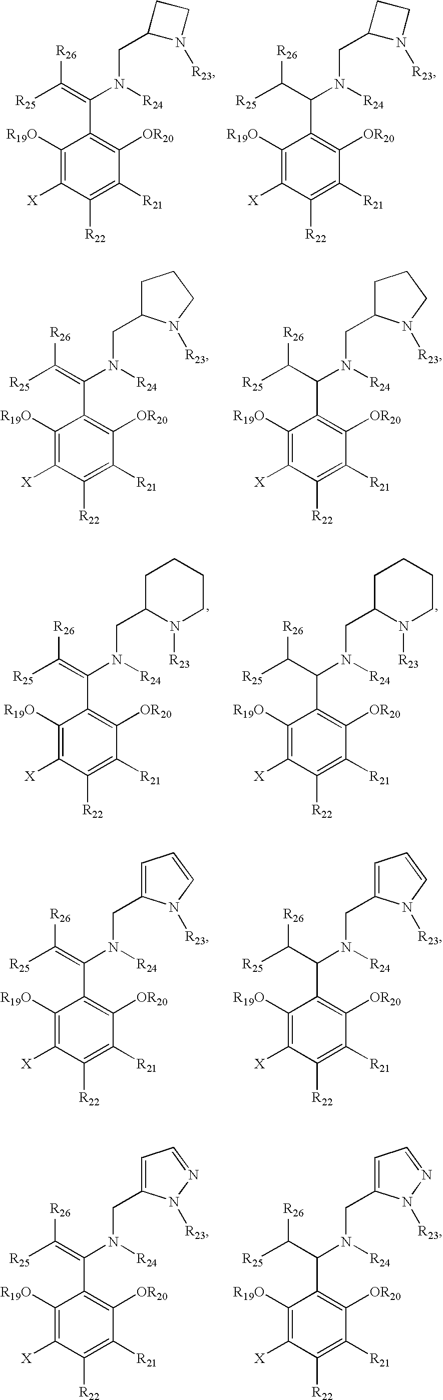 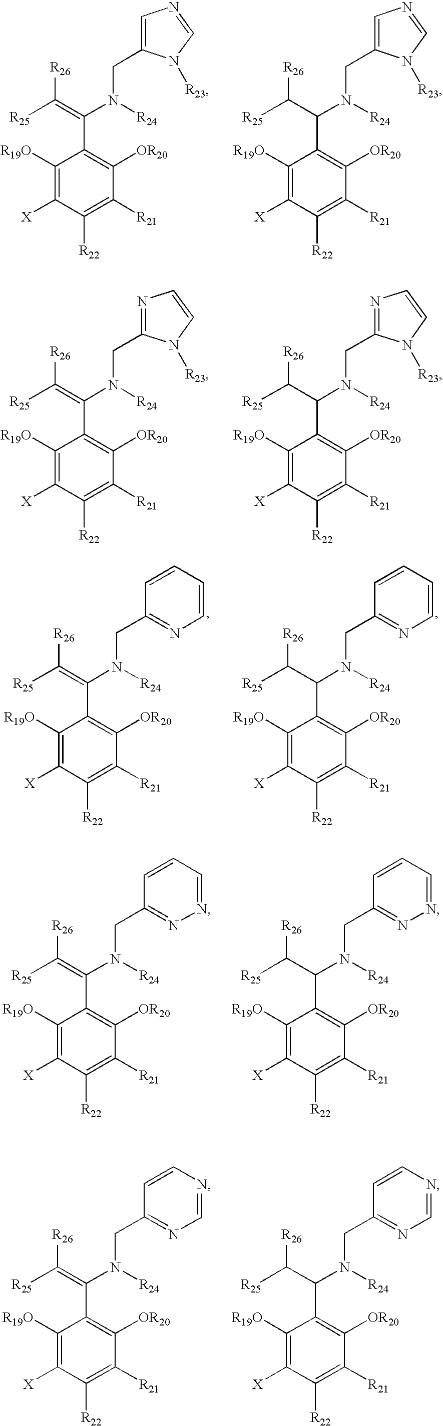 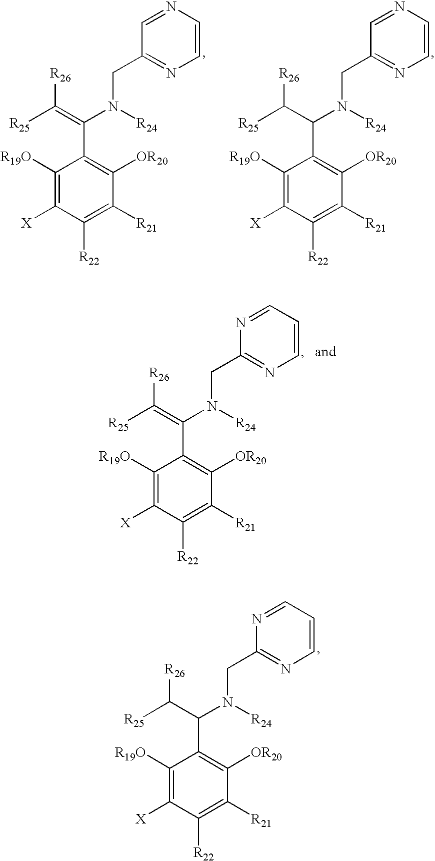
tautomers, stereoisomers, and geometric isomers thereof, and pharmaceutical derivatives thereof,
wherein R₂₃ selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

18. The compound of claim 17, wherein said compound has a structure selected from the group consisting of 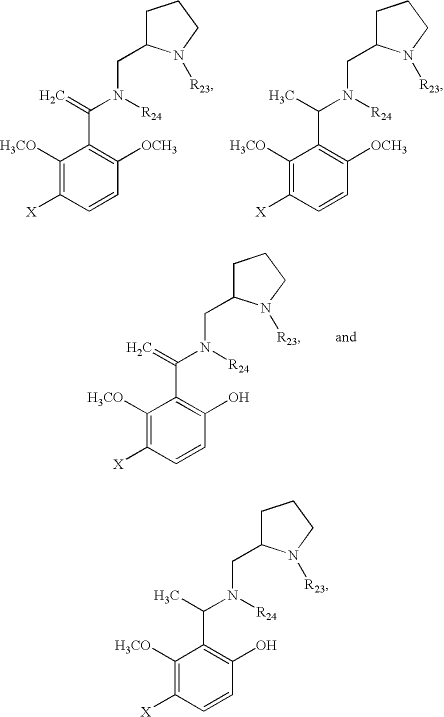
tautomers, stereoisomers, and geometric isomers thereof, and pharmaceutical derivatives thereof.

19. The compound of claim 18, wherein said compound has a structure selected from the group consisting of 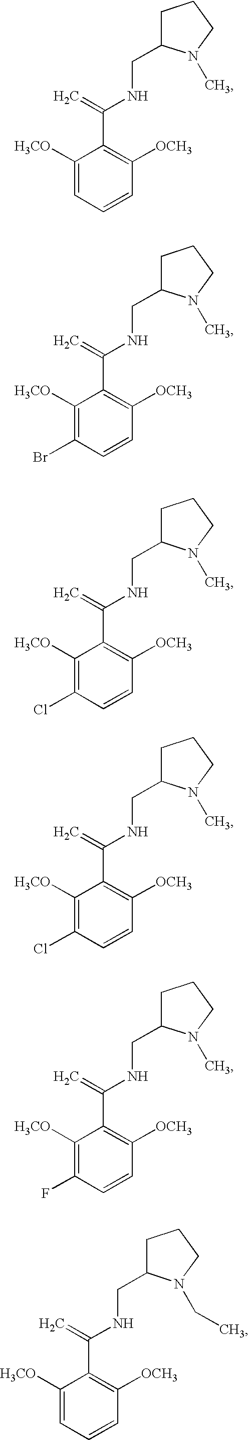 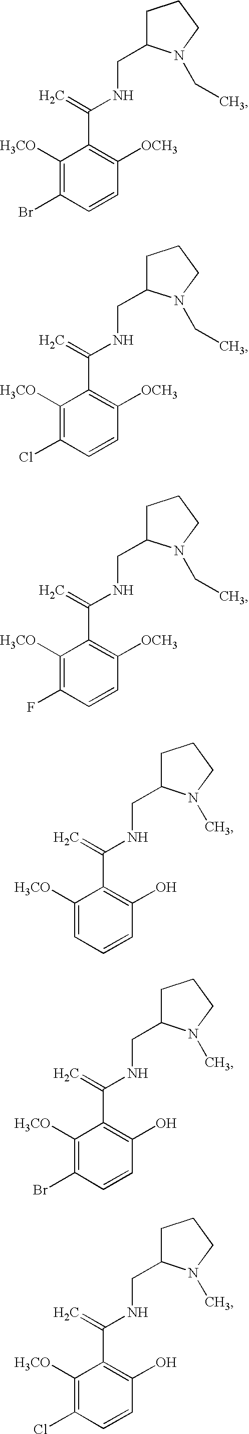 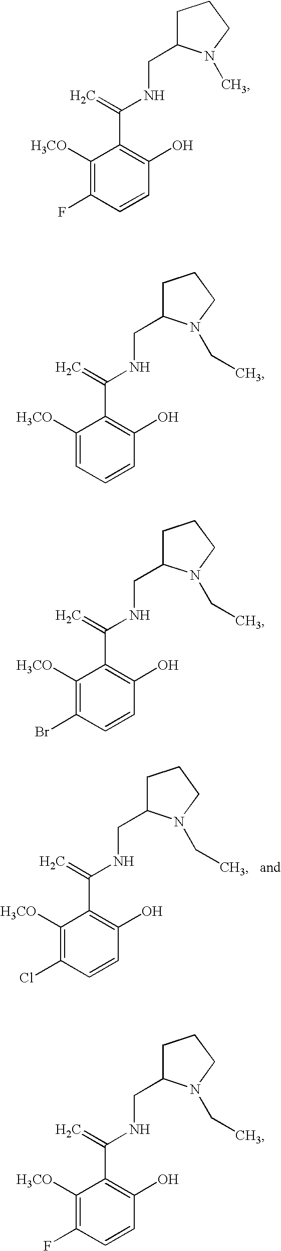
tautomers, stereoisomers, and geometric isomers thereof, and pharmaceutical derivatives thereof.

20. The compound of claim 18, wherein said compound has a structure selected from the group consisting of 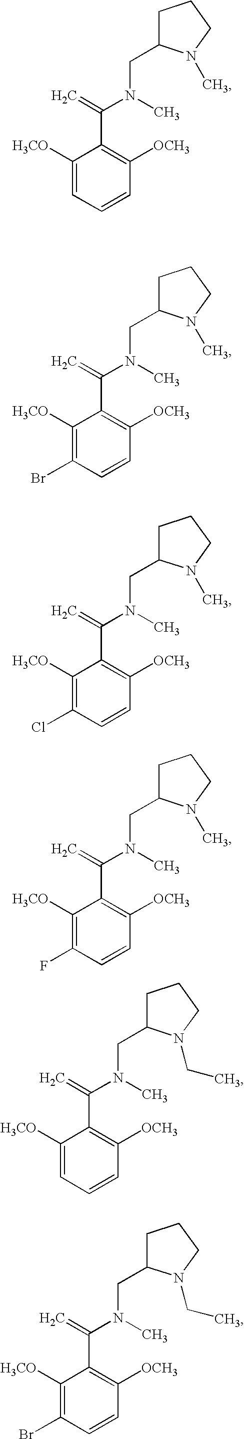 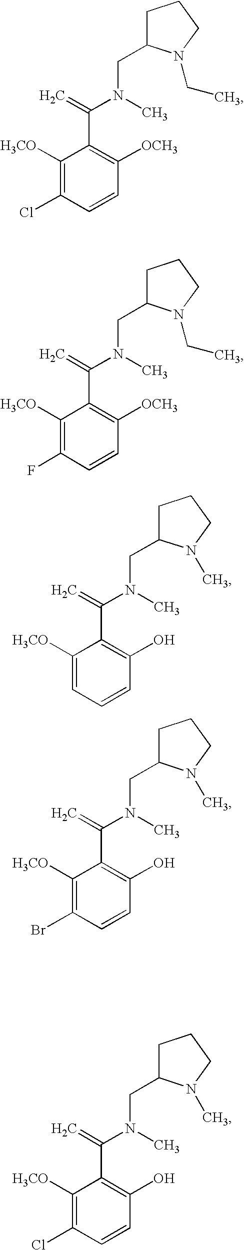 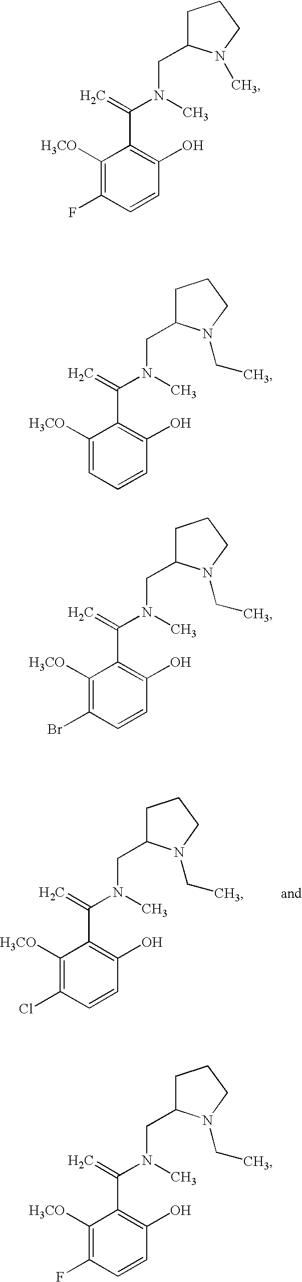
stereoisomers and pharmaceutical derivatives thereof.

21. The compound of claim 18, wherein said compound has a structure selected from the group consisting of 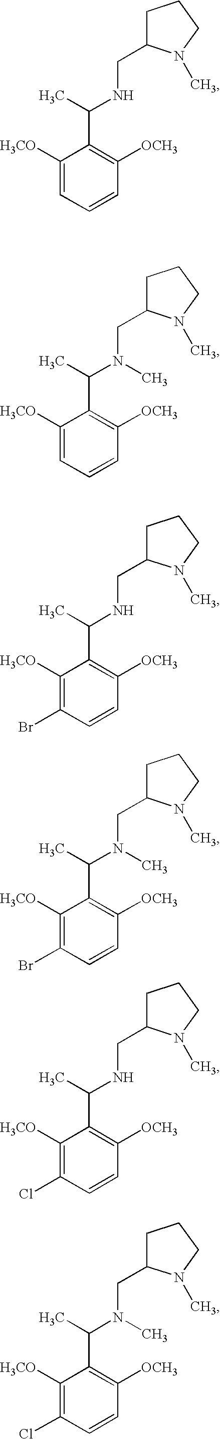 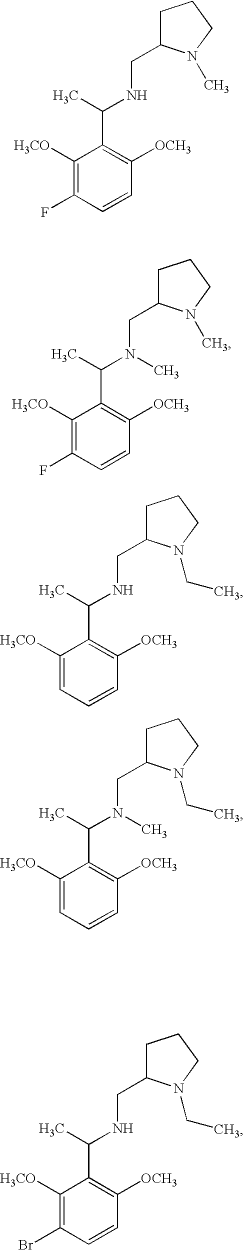 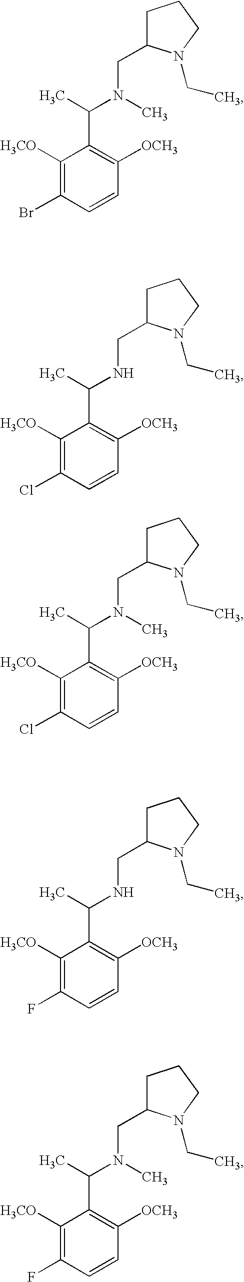 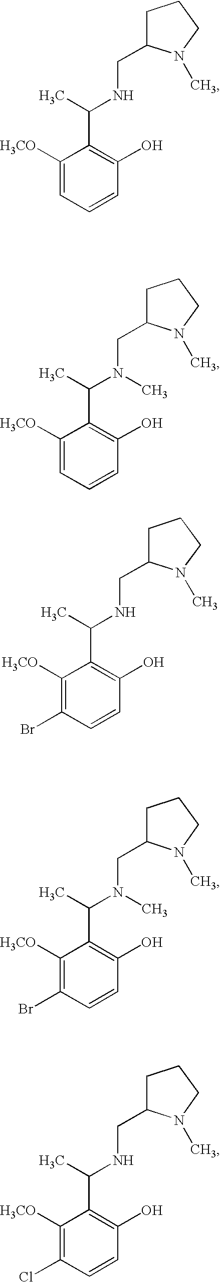 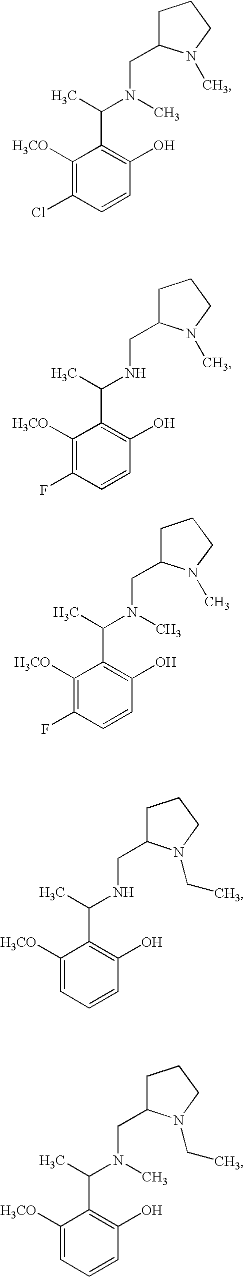 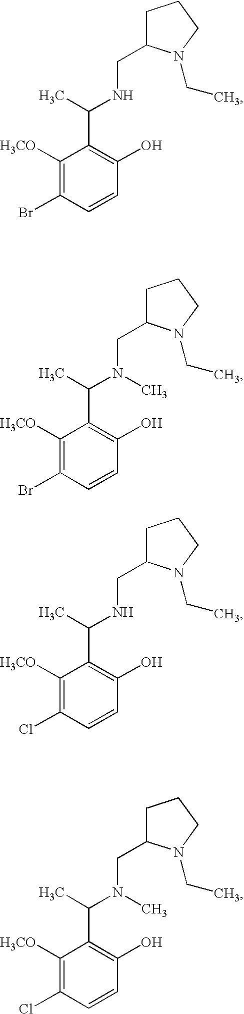 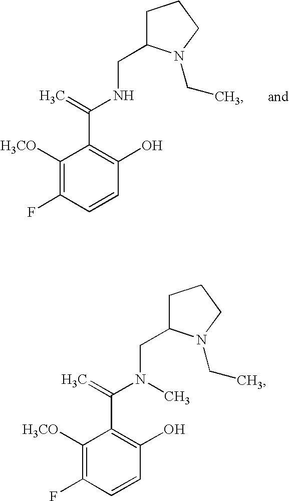
stereoisomers and pharmaceutical derivatives thereof.

22. A compound having a structure of: 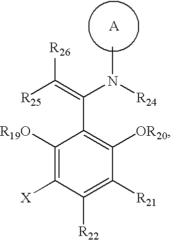
tautomers, stereoisomers, and geometric isomers thereof, and pharmaceutical derivatives thereof,
wherein A is a ring selected from the group consisting of a heterocyclic ring and a heteroaromatic ring,
wherein 3 to 9 carbon atoms and 1 to 4 nitrogen atoms comprise said ring;
wherein X is selected from the group consisting of H, F, Cl, and Br;
wherein R₁₉ and R₂₀ are independently selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, and heteroarylalkylcarbonyl;
wherein R₂₁ and R₂₂ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio;
wherein R₂₄ is selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl; and
wherein R₂₅ and R₂₆ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

23. The compound of claim 22, wherein said compound has a structure selected from the group consisting of 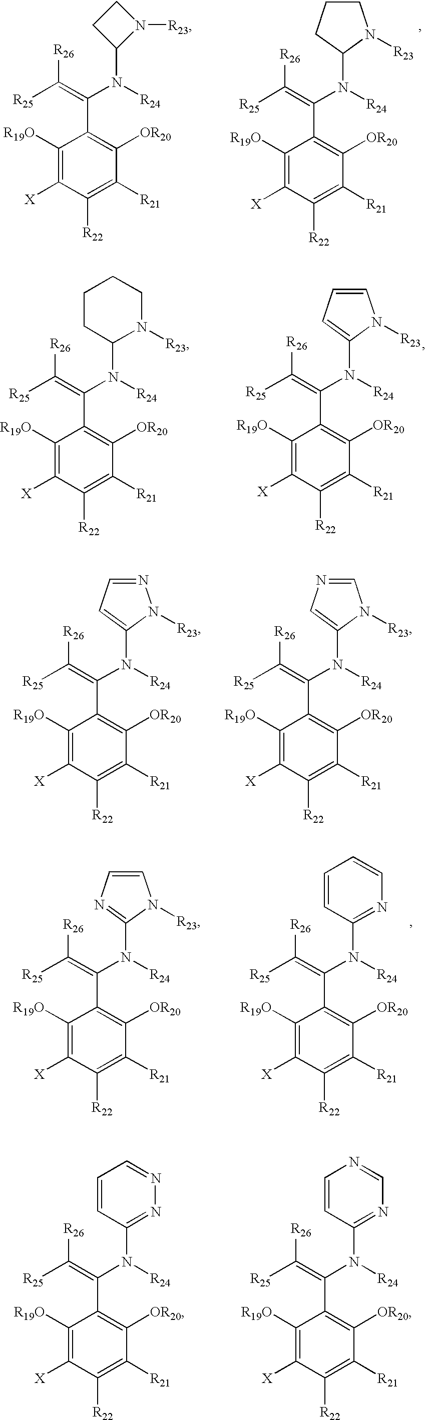 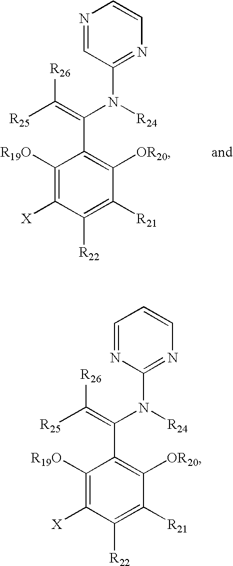
tautomers, stereoisomers, and geometric isomers thereof, and pharmaceutical derivatives thereof,
wherein R₂₃ selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

24. The compound of claim 23, wherein said compound has a structure selected from the group consisting of 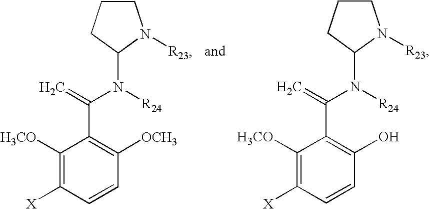
tautomers and stereoisomers thereof, and pharmaceutical derivatives thereof.

25. The compound of claim 24, wherein said compound has a structure selected from the group consisting of: 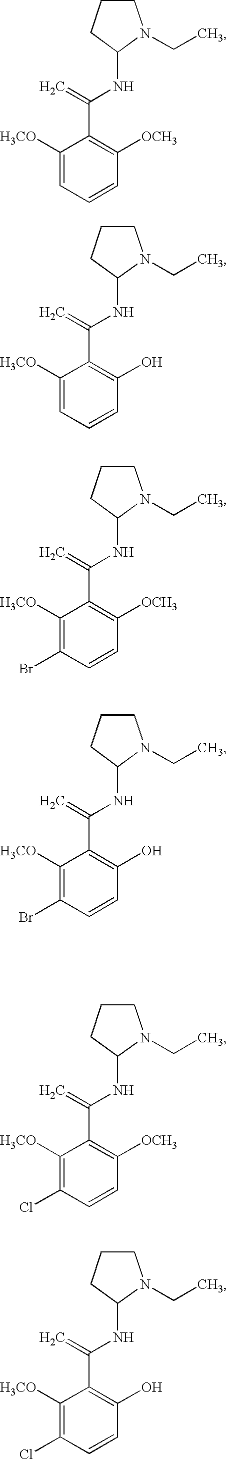 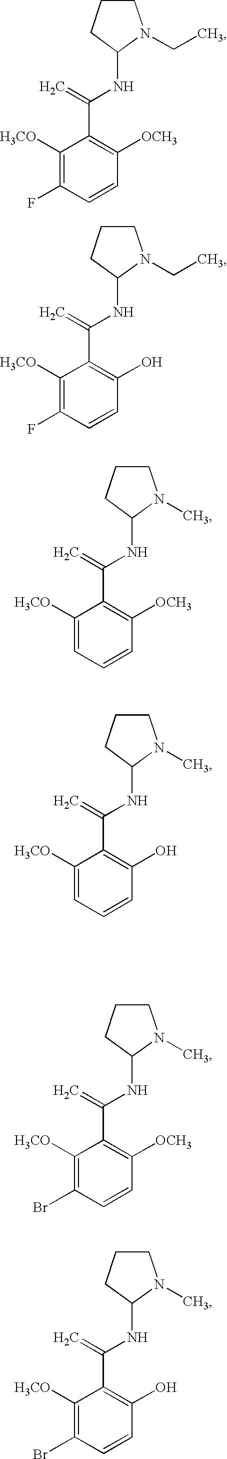 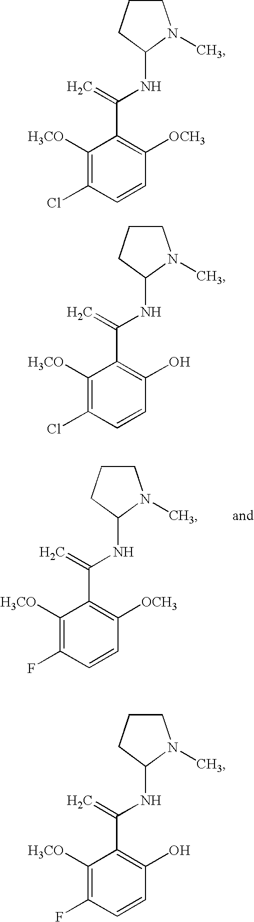
tautomers and stereoisomers thereof, and pharmaceutical derivatives thereof.

26. The compound of claim 24, wherein said compound has a structure selected from the group consisting of 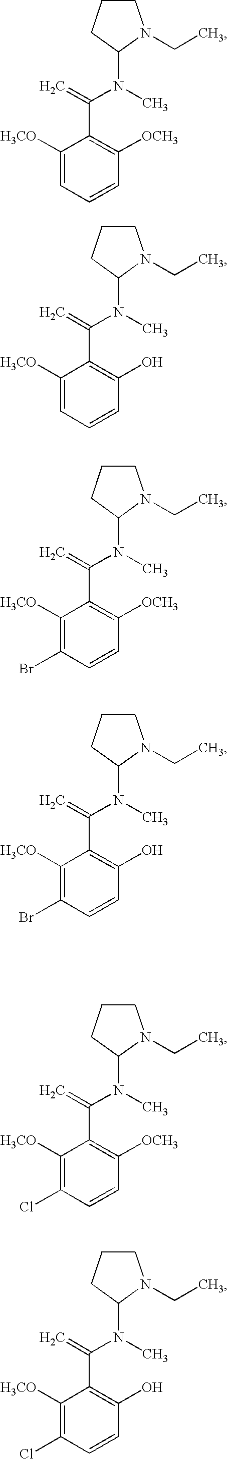 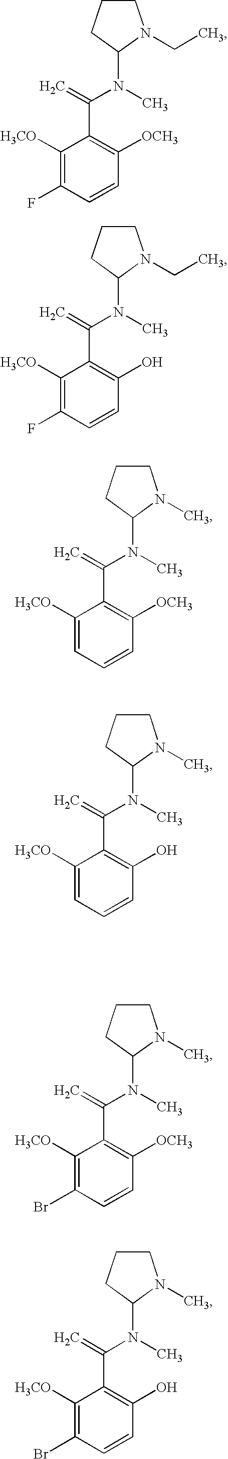 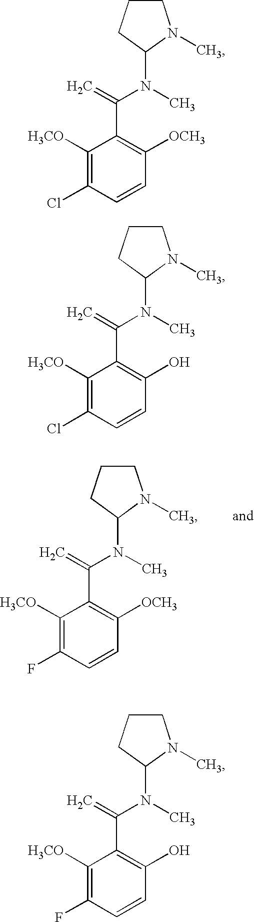
stereoisomers thereof, and pharmaceutical derivatives thereof.

27. A compound having a structure of: 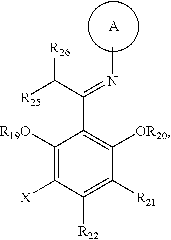
tautomers, stereoisomers, and geometric isomers thereof, and pharmaceutical derivatives thereof,
wherein A is a ring selected from the group consisting of a heterocyclic ring and a heteroaromatic ring,
wherein 3 to 9 carbon atoms and 1 to 4 nitrogen atoms comprise said ring;
wherein X is selected from the group consisting of H, F, Cl, and Br;
wherein R₁₉ and R₂₀ are independently selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, and heteroarylalkylcarbonyl;
wherein R₂₁ and R₂₂ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio; and
wherein R₂₅ and R₂₆ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio.

28. The compound of claim 27, wherein said compound has a structure selected from the group consisting of 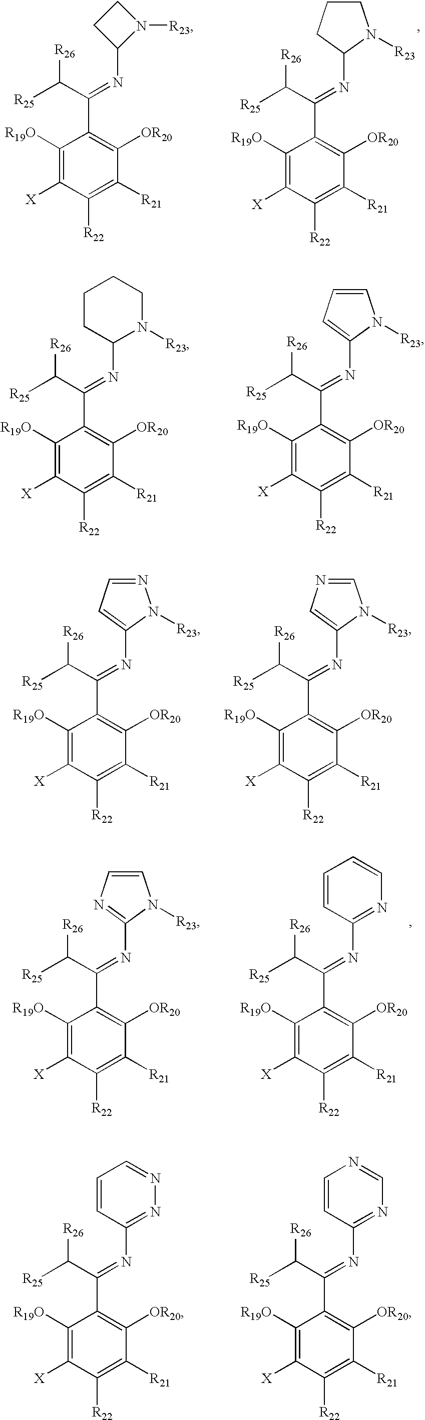 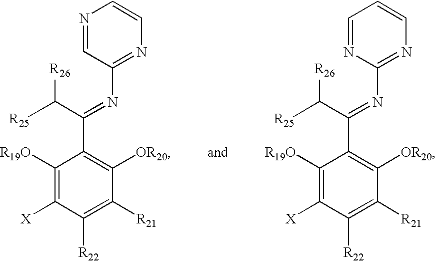
tautomers, stereoisomers, and geometric isomers thereof, and pharmaceutical derivatives thereof,
wherein R₂₃ selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, heteroalkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkylsulfonyl, alkenylsulfonyl, alkynylsulfonyl, arylsulfonyl, aralkylsulfonyl, heteroalkylsulfonyl, cycloalkylsulfonyl, cycloalkylalkylsulfonyl, heterocyclylsulfonyl, heterocyclylalkylsulfonyl, heteroarylsulfonyl, and heteroarylalkylsulfonyl.

29. The compound of claim 28, wherein said compound has a structure selected from the group consisting of 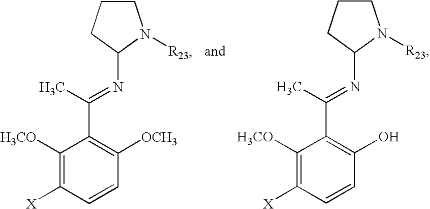
tautomers, stereoisomers, and geometric isomers thereof, and pharmaceutical derivatives thereof.

30. The compound of claim 29, wherein said compound has a structure selected from the group consisting of 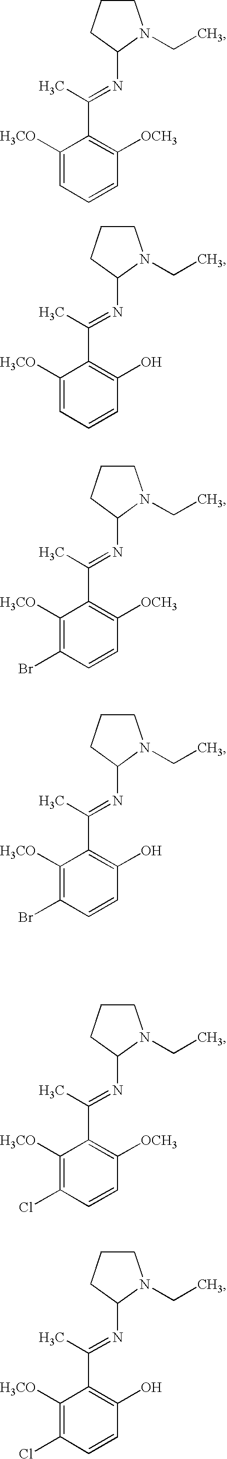 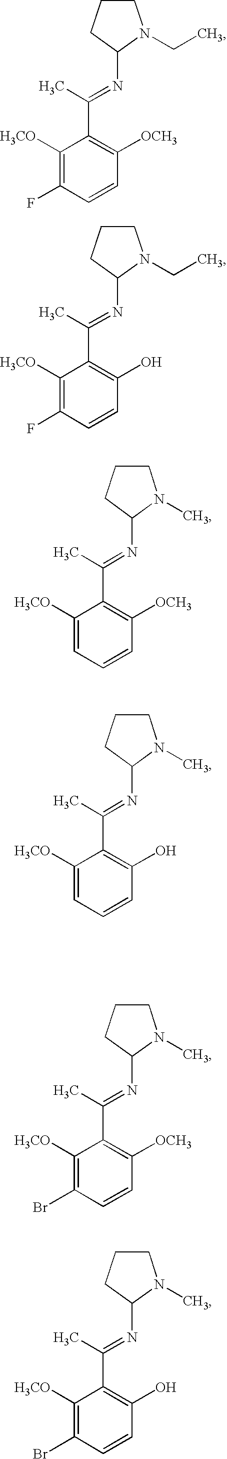 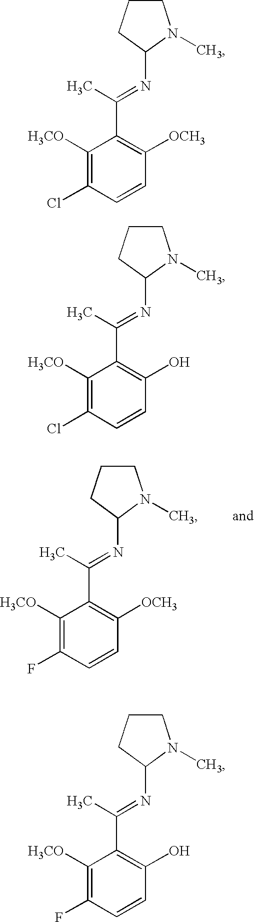
tautomers, stereoisomers, and geometric isomers thereof, and pharmaceutical derivatives thereof.

31. A compound having a structure of: 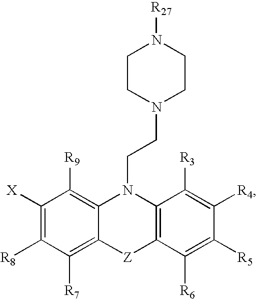
and pharmaceutical derivatives thereof,
wherein X is selected from the group consisting of H, F, Cl, and Br;
wherein Z is selected from the group consisting of NR₁₂, N(C═O)R₁₂, NNH(C═O)R₁₃, NSO₂R₁₃, S, SO₂, O, CR₁₄R₁₅, C═O, C═CR₁₄R₁₅, and PR₁₂;
wherein R₁₂ is selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₃ is selected from the group consisting of alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₄ and R₁₅ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio;
wherein R₃-R₉ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′-dialkyl-N-arylureido, N-alkyl-N′,N′-diarylureido, N-aryl-N′,N′-dialkylureido, alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio;
wherein R₂₇ is selected from the group consisting of H, C1-C4 alkyl, and phenyl,
wherein said C1-C4 alkyl comprises methyl, ethyl, propyl, isopropyl, butyl, s-butyl, t-butyl, and isobutyl; and
wherein the selection of X, Z, R₃-R₉ and R₂₇ is conducted with the proviso that none of the following conditions are met:
i) X is H, Z is S, R₂₇ is selected from the group consisting of H, methyl, ethyl, propyl, butyl, and phenyl, and R₃-R₉ are all H;
ii) X is H, Z is S, R₄ is methoxy, and R₃, R₅-R₉ and R₂₇ are all H;
iii) X is H, Z is S, R₃ and R₇-R₉ are all H, any R₄-R₆ that are not Cl, methyl, ethyl, mercaptomethyl, methylcarbonyl, ethylcarbonyl, propylcarbonyl, methoxycarbonyl, or trifluoromethyl are all H, R₂₇ is selected from the group consisting of H, methyl, ethyl, and butyl, and at least one of R₄-R₆ is selected from the group consisting of Cl, methyl, ethyl, mercaptomethyl, methylcarbonyl, ethylcarbonyl, propylcarbonyl, methoxycarbonyl, and trifluoromethyl;
iv) X is H, Z is SO₂, R₃-R₉ are all H, and R₂₇ is selected from the group consisting of H, methyl, ethyl, isobutyl, and t-butyl;
v) X is H, Z is O, R₃-R₉ are all H, and R₂₇ is selected from the group consisting of H, methyl, and ethyl;
vi) X is H, Z is O, R₃ and R₅-R₉ are all H, R₂₇ is methyl, and R₄ is ethyl;
vii) X is Cl, Z is S, R₃-R₉ are all H, and R₂₇ is selected from the group consisting of H, methyl, ethyl, and butyl;
viii) X is Cl, Z is S, R₃-R₆, R₈ and R₉ are all H, R₂₇ is methyl, and R₇ is Cl; and
ix) X is H, Z is C═O, R₃-R₉ are all H, and R₂₇ is selected from the group consisting of methyl and propyl.

32. The compound of claim 31, wherein X is selected from the group consisting of F, Cl, and Br, and Z is selected from the group consisting of NR₁₂, N(C═O)R₁₂, NNH(C═O)R₁₃, NSO₂R₁₃, O, CR₁₄R₁₅, C═O, C═CR₁₄R₁₅, and PR₁₂.

33. The compound of claim 31, wherein said compound has a structure selected from the group consisting of 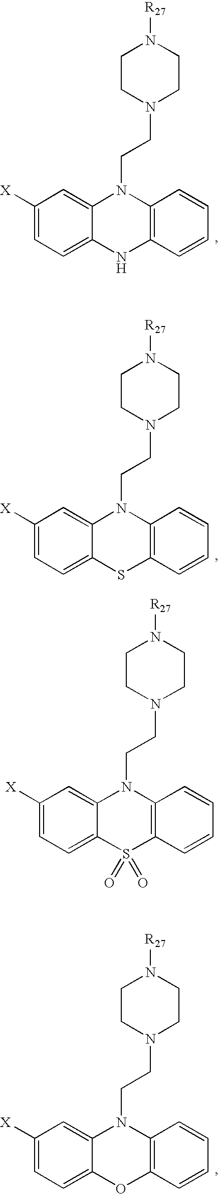 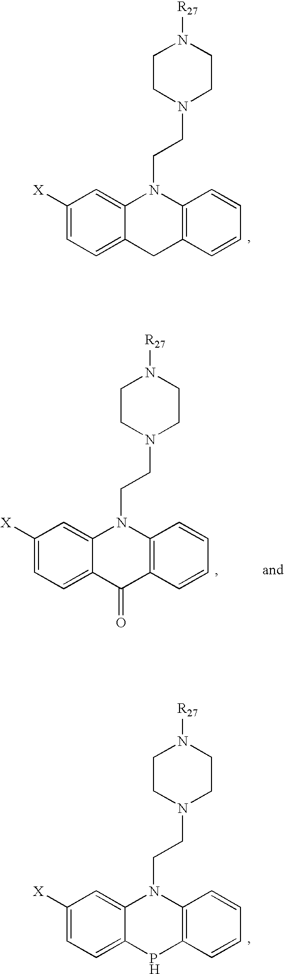
and pharmaceutical derivatives thereof.

34. The compound of claim 33, wherein said compound has a structure selected from the group consisting of 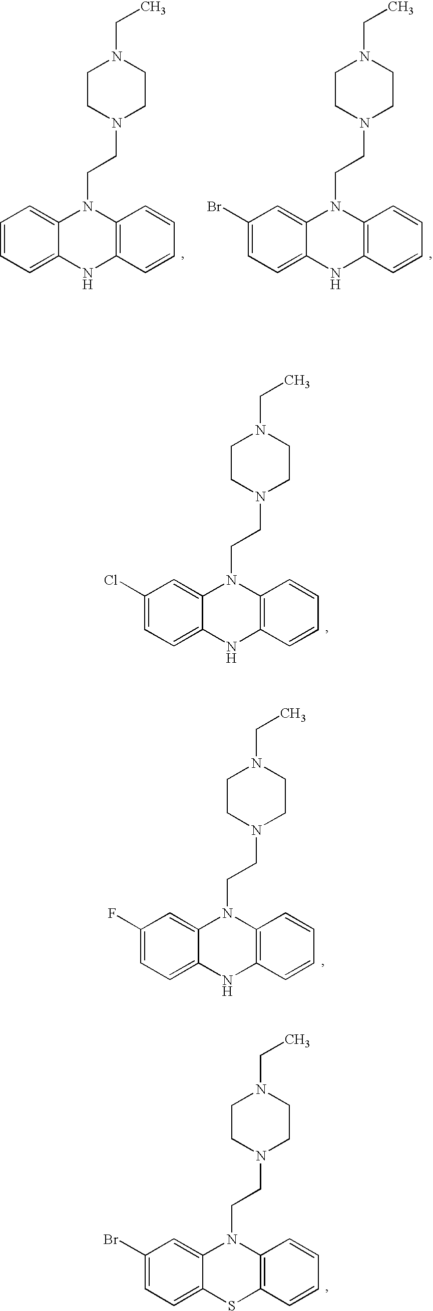 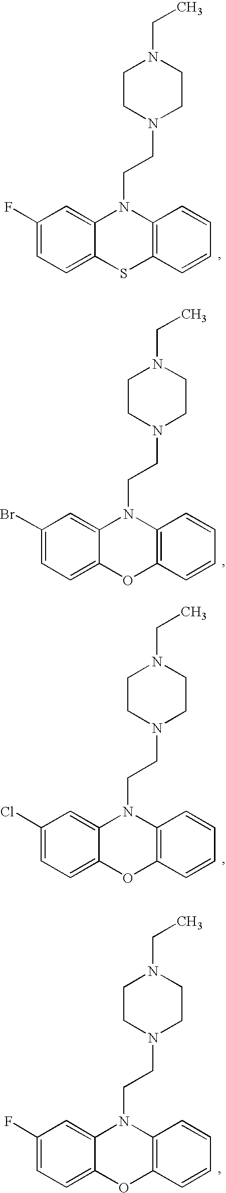 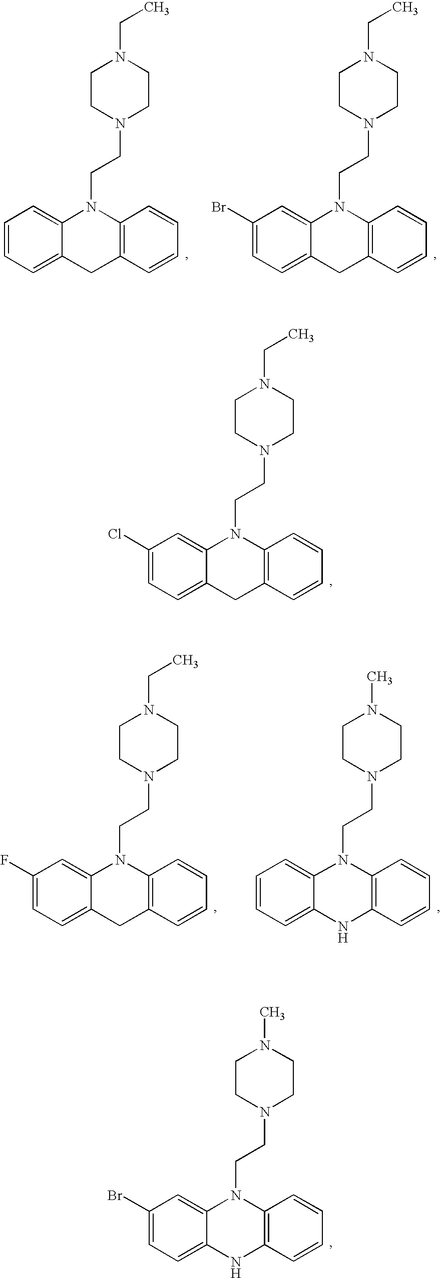 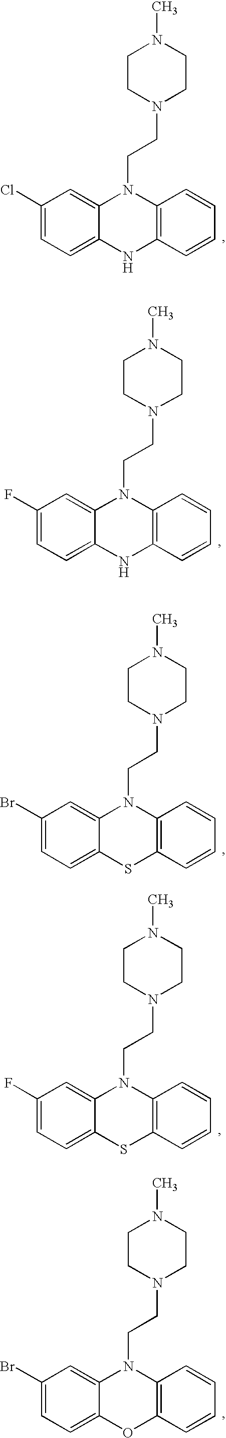 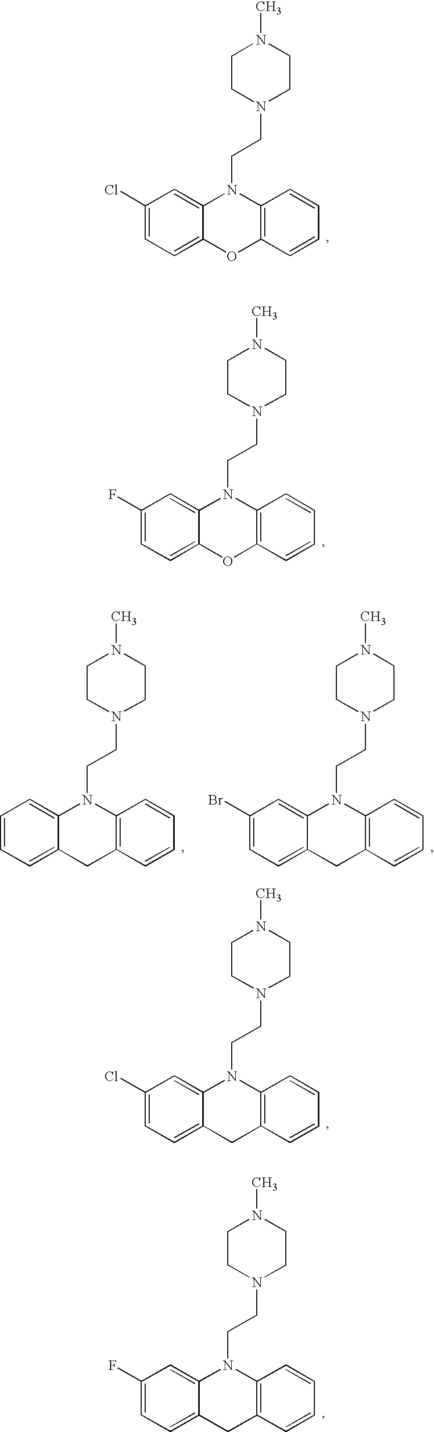 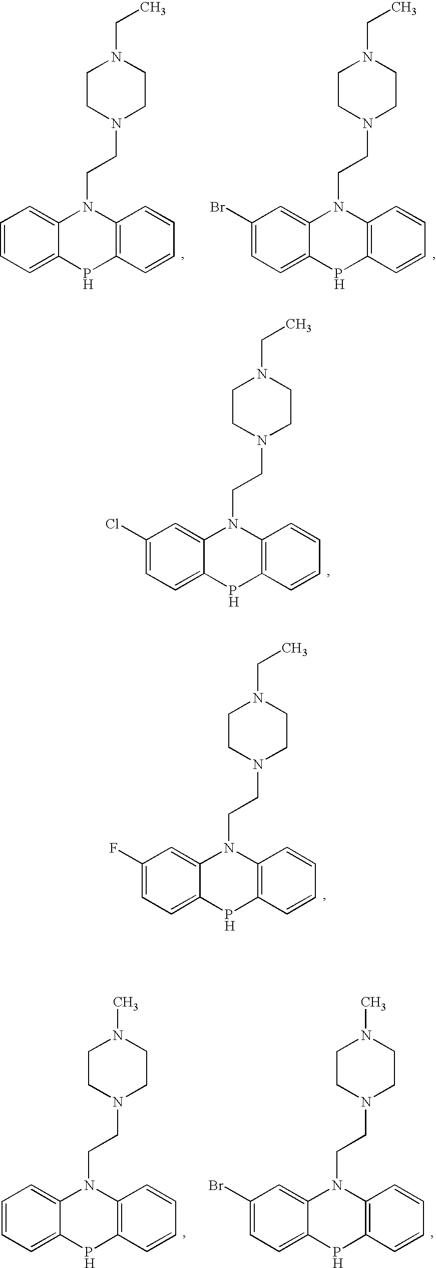 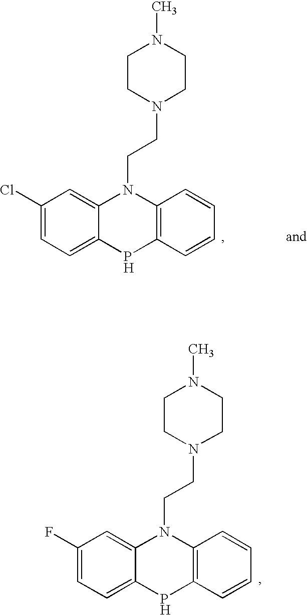
and pharmaceutical derivatives thereof.

35. A compound having a structure of: 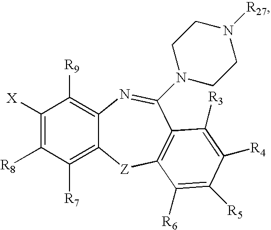
and pharmaceutical derivatives thereof,
wherein X is selected from the group consisting of F and Br;
wherein Z is selected from the group consisting of S, SO₂, O, CR₁₄R₁₅, C═O, C═CR₁₄R₁₅, and PR₁₂;
wherein R₁₂ is selected from the group consisting of H, alkyl, alkenyl, alkynyl, heteroalkyl, aryl, aralkyl, cycloalkyl, cycloalkylalkyl, heterocyclyl, heterocyclylalkyl, heteroaryl, and heteroarylalkyl;
wherein R₁₄ and R₁₅ are independently selected from the group consisting of H, halo, pseudohalo, alkyl, alkenyl, alkynyl, heteroalkyl, aryl, aralkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, heteroalkylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, alkylsulfonylamino, arylsulfonylamino, alkylsulfonylaminoalkyl, alkylsulfonylaminoaryl, arylaminosulfonylaryl, cyano, nitro, amino, alkylamino, dialkylamino, arylamino, diarylamino, alkylarylamino, alkylcarbonylamino, arylcarbonylamino, alkylcarbonylaminoalkyl, alkylcarbonylaminoaryl, arylcarbonylaminoaryl, hydroxy, carboxy, alkylcarbonyloxy, arylcarbonyloxy, alkoxy, alkenyloxy, alkynyloxy, heteroalkoxy, aryloxy, aralkoxy, cycloalkyloxy, cycloalkylalkoxy, heterocyclyloxy, heterocyclylalkoxy, heteroaryloxy, heteroarylalkoxy, perfluoroalkoxy, alkyl and aryl ammonium salts, thioxy, alkylthio, and arylthio;
wherein R₃-R₉ are independently selected from the group consisting of H, alkyl, alkenyl, alkynyl, aryl, aralkyl, heteroalkyl, cycloalkyl, cycloalkylalkyl, heterocyclyl, heterocyclylalkyl, heteroaryl, heteroarylalkyl, alkylcarbonyl, alkenylcarbonyl, alkynylcarbonyl, arylcarbonyl, aralkylcarbonyl, cycloalkylcarbonyl, cycloalkylalkylcarbonyl, heterocyclylcarbonyl, heterocyclylalkylcarbonyl, heteroarylcarbonyl, heteroarylalkylcarbonyl, alkoxycarbonyl, alkenyloxycarbonyl, alkynyloxycarbonyl, heteroalkoxycarbonyl, aryloxycarbonyl, aralkoxycarbonyl, cycloalkoxycarbonyl, cycloalkylalkoxycarbonyl, heterocyclyloxycarbonyl, heterocyclylalkoxycarbonyl, heteroaryloxycarbonyl, heteroarylalkoxycarbonyl, alkylsulfonyl, alkenylsulfonyl, alkynylsulfonyl, heteroalkylsulfonyl, arylsulfonyl, aralkylsulfonyl, cycloalkylsulfonyl, cycloalkylalkylsulfonyl, heterocyclylsulfonyl, heterocyclylalkylsulfonyl, heteroarylsulfonyl, heteroarylalkylsulfonyl, halo, pseudohalo, hydroxy, alkylcarbonyloxy, arylcarbonyloxy, cyano, nitro, mercapto, haloalkyl, carboxy, perfluoroalkyl, amino, alkylamino, dialkylamino, arylamino, diarylamino, alkylarylamino, alkoxy, alkenyloxy, alkynyloxy, heteroalkyloxy, aryloxy, aralkoxy, cycloalkyloxy, cycloalkylalkyloxy, heterocyclyloxy, heterocyclylalkoxy, heteroaryloxy, heteroarylalkoxy, perfluoroalkoxy, aminocarbonyl, alkylaminocarbonyl, dialkylaminocarbonyl, arylaminocarbonyl, diarylaminocarbonyl, arylalkylaminocarbonyl, ureido, N-alkylureido, N-arylureido, N′-alkylureido, N′-aryluredio, N,N′-dialkyureido, N,N′-diarylureido, N′-arylureido, N,N′-diarylureido, N,N′,N′-trialkylureido, N,N′-dialkyl-N-arylureido, N-alkyl-N′,N′-diarylureido, N-aryl-N′,N′-dialkylureido, N,N′-diaryl-N′-alkylureido, N,N′,N′-triarylureido, thioureido, thioxy, alkylthio, arylthio, aminosulfonyl, alkylaminosulfonyl, arylaminosulfonyl, dialkylaminosulfonyl, diarylaminosulfonyl, alkylarylaminosulfonyl, guanidino, isothioureido, amidino, alkylsulfonylamino, arylsulfonylamino, heteroarylsulfonylamino, alkylcarbonylamino, arylcarbonylamino, alkylcarbonylaminoalkyl, alkylcarbonylaminoaryl, arylcarbonylaminoalkyl, arylcarbonylamainoaryl, alkyl and aryl ammonium salts, and perfluoroalkylthio;
wherein R₂₇ is selected from the group consisting of H, C1-C4 alkyl, and phenyl,
wherein said C1-C4 alkyl comprises methyl, ethyl, propyl, isopropyl, butyl, s-butyl, t-butyl, and isobutyl; and
wherein the selection of X, Z, R₃-R₉ and R₂₇ is conducted with the proviso that none of the following conditions are met:
i) X is F, Z is O, R₃ and R₅-R₉ are all H, R₂₇ is selected from the group consisting of H, methyl, ethyl, propyl, and R₄ is selected from the group consisting of H, methyl, mercaptomethyl, bromo, amino, and nitro;
ii) X is F, Z is O, R₃ and R₅-R₉ are all H, R₂₇ is methyl, and R₄ is nitro;
iii) X is F, Z is O, R₃-R₅, R₇-R₉ and R₂₇ are all H, and R₆ is methyl;
iv) X is F, Z is O, R₃-R₆ are all H, R₂₇ is selected from the group consisting of methyl and phenyl, and R₇-R₉ are all F;
v) X is F, Z is S, R₃ and R₅-R₉ are all H, R₂₇ is selected from the group consisting of methyl, ethyl, and propyl, and R₄ is selected from the group consisting of H, mercaptomethyl, chloro, and bromo;
vi) X is Br, Z is O, R₃ and R₅-R₉ are all H, R₂₇ is selected from the group consisting of H, methyl, ethyl, and propyl, and R₄ is selected from the group consisting of H, mercaptomethyl, bromo, and trifluoromethyl;
vii) X is Br, Z is S, R₃ and R₅-R₉ are all H, R₂₇ is selected from the group consisting of methyl and ethyl, and R₄ is mercaptomethyl;
viii) X is Br, Z is O, R₃, R₄, R₆-R₉, and R₂₇ are all H, and R₅ is methoxy;
ix) X is Br, Z is O, R₃-R₅, R₇-R₉, and R₂₇ are all H, and R₆ is methyl;
x) X is F, Z is CR₁₄R₁₅, R₃-R₇, R₉, R₁₄, and R₁₅ are all H, R₂₇ is selected from the group consisting of H, methyl, isopropyl, and t-butyl, and Rₐ is selected from the group consisting of H, hydroxy, methyl, fluoro, chloro, and sulfonyloxy; and
xi) X is F, Z is C═CR₁₄R₁₅, R₃-R₉, R₁₄, and R₁₅ are all H, and R₂₇ is selected from the group consisting of H and methy

36. The compound of claim 35, wherein said compound has a structure selected from the group consisting of 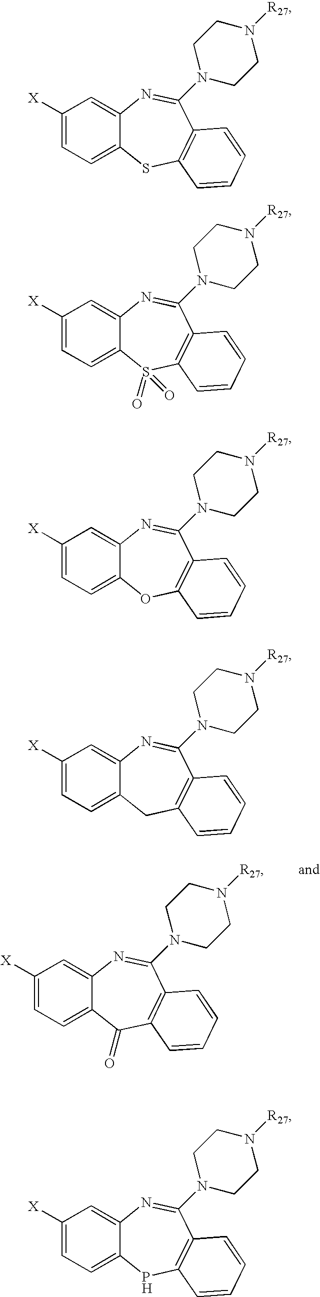
and pharmaceutical derivatives thereof.

37. The compound of claim 36, wherein said compound has a structure selected from the group consisting of 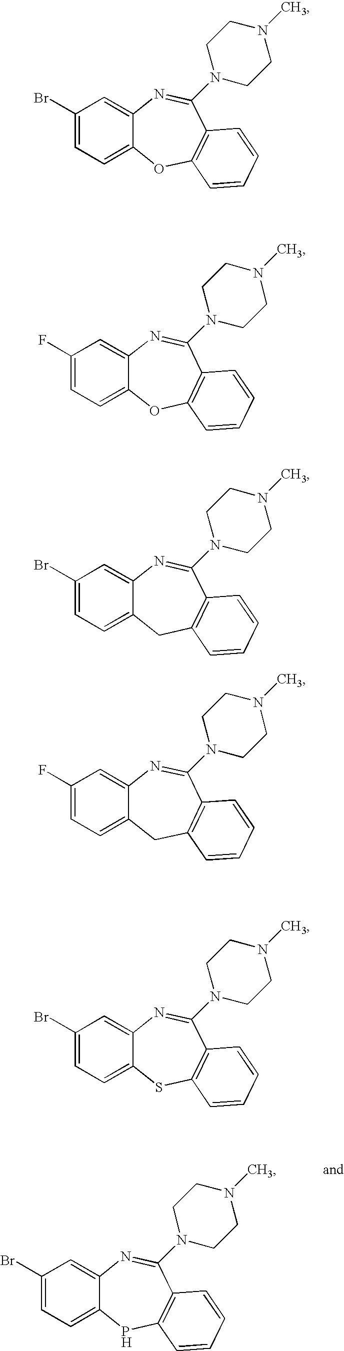 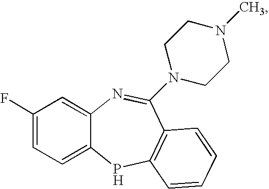
and pharmaceutical derivatives thereof.
